(19) United States
(12) Patent Application Publication
(10) Pub. No.: US 2025/0388646 A1
(43) Pub. Date: Dec. 25, 2025

(54) ENHANCING EFFECTOR CELL DURABILITY AND EFFICACY IN ADOPTIVE CELL THERAPIES

(71) Applicant: FATE THERAPEUTICS, INC., San Diego, CA (US)

(72) Inventors: Eigen PERALTA, San Diego, CA (US); Christopher Michael ECKER, San Diego, CA (US); Bahram VALAMEHR, San Diego, CA (US); Hui-Yi CHU, San Diego, CA (US); Tom Tong LEE, San Diego, CA (US)

(21) Appl. No.: 18/878,845

(22) PCT Filed: Jun. 29, 2023
(86) PCT No.: PCT/US2023/069412

§ 371 Date: Dec. 24, 2024

Related U.S. Application Data
(60) Provisional application No. 63/367,596, filed on Jul. 1, 2022.

(51) Int. Cl. C07K 14/705; A61K 38/20; A61K 40/11; A61K 40/31; A61K 40/42; C07K 14/47; C07K 14/715; C07K 16/28; C12N 5/0783

(52) U.S. Cl. CPC C07K 14/70578; A61K 38/2013; A61K 38/2086; A61K 40/11; A61K 40/31; A61K 40/4211; C07K 14/47; C07K 14/7155; C07K 16/2803; C12N 5/0636; C07K 2319/02; C07K 2319/03; C12N 2510/00

(57) ABSTRACT

Provided are methods and compositions for obtaining functionally enhanced derivative effector cells obtained from directed differentiation of genomically engineered iPSCs. Embodiments of derivative cells provided herein have stable and functional genome editing that delivers improved or enhanced therapeutic effects. Also provided are therapeutic compositions and the use thereof comprising the functionally enhanced derivative effector cells alone, or with antibodies or checkpoint inhibitors in combination therapies.

### CROSS-REFERENCE TO RELATED APPLICATION

This application claims the benefit of priority to U.S. Provisional Application No. 63/367,596, filed Jul. 1, 2022, which is hereby incorporated by reference in its entirety.

### INCORPORATION BY REFERENCE OF SEQUENCE LISTING

The instant application contains a Sequence Listing, which has been submitted electronically. The Sequence Listing titled 184143-644601_SL.xml, which was created on Jun. 28, 2023 and is 30,164 bytes in size, is hereby incorporated by reference in its entirety.

### FIELD OF THE INVENTION

The present disclosure is broadly concerned with the field of off-the-shelf immunocellular products. More particularly, the present disclosure is concerned with strategies for developing multifunctional effector cells capable of delivering therapeutically relevant properties in vivo. The cell products developed under the present disclosure address critical limitations of patient-sourced cell therapies.

### BACKGROUND OF THE INVENTION

The field of adoptive cell therapy is currently focused on using patient- and donor-sourced cells, which makes it particularly difficult to achieve consistent manufacturing of cancer immunotherapies and to deliver therapies to all patients who may benefit therefrom. There is also a need to improve the efficacy and persistence of adoptively transferred lymphocytes to promote favorable patient outcomes. Lymphocytes such as T cells and natural killer (NK) cells are potent anti-tumor effectors that play an important role in innate and adaptive immunity. However, the use of these immune cells for adoptive cell therapies remains challenging and has unmet needs for improvement. Therefore, there remain significant opportunities to harness the full potential of T and NK cells, or other lymphocytes in adoptive immunotherapy.

### SUMMARY OF THE INVENTION

There is a need for functionally improved effector cells that address issues ranging from response rate, cell exhaustion, loss of transfused cells (survival and/or persistence), tumor escape through target loss or lineage switch, tumor targeting precision, off-target toxicity, off-tumor effect, to efficacy against solid tumors, i.e., tumor microenvironment and related immune suppression, recruiting, trafficking and infiltration.

It is an object of embodiments of the present invention to provide methods and compositions to generate derivative non-pluripotent cells differentiated from a single cell derived iPSC (induced pluripotent stem cell) clonal line, which iPSC line comprises one or several genetic modifications in its genome. Said one or several genetic modifications include, in some embodiments, DNA insertion, deletion, and substitution, and which modifications are retained and remain functional in subsequently derived cells after differentiation, expansion, passaging and/or transplantation.

iPSC-derived non-pluripotent cells of the present application include, but are not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells. In some embodiments, iPSC-derived non-pluripotent cells of the present application comprise one or several genetic modifications in their genome through differentiation from an iPSC comprising the same genetic modifications. In some embodiments, the engineered clonal iPSC differentiation strategy for obtaining genetically engineered derivative cells benefits from the developmental potential of the iPSC in a directed differentiation that is not significantly adversely impacted by the engineered modality in the iPSC, and also that the engineered modality functions as intended in the derivative cell. Further, this strategy overcomes the present barrier in engineering primary lymphocytes, such as T cells or NK cells obtained from peripheral blood, as such cells are difficult to engineer, with engineering of such cells often lacking reproducibility and uniformity, resulting in cells exhibiting poor cell persistence with high cell death and low cell expansion. Moreover, this strategy avoids production of a heterogenous effector cell population otherwise obtained using primary cell sources which are heterogenous to start with.

Accordingly, in one aspect, the present invention also provides a cell or population thereof, wherein: (i) the cell is (a) an immune cell; (b) an induced pluripotent cell (iPSC), a clonal iPSC, or an iPS cell line cell; or (c) a derivative cell obtained from differentiating the cell of (b); (ii) the cell comprises an exogenous polynucleotide encoding a signaling redirector receptor that comprises (a) an extracellular Fas binding domain comprising a partial or full peptide of an extracellular domain (ECD) of a Fas receptor (FAS), or a variant or an allele thereof, and (b) a cytoplasmic signaling domain comprising a partial or full peptide of an intracellular domain (ICD) of one or more co-stimulation molecules, wherein the signaling redirector receptor is a Fas redirector that redirects Fas signaling upon binding to a FAS agonist, thereby providing the cell or the derivative cell with improved apoptosis resistance and/or exhaustion resistance. In various embodiments, (i) the Fas redirector further comprises a transmembrane region comprising a transmembrane domain, or a portion thereof, of a transmembrane protein; (ii) the one or more co-stimulation molecules comprise CD27, CD28, CD40, MyD88, OX40, IL12Rβ2, IL18R1, IL21R, or a combination thereof, (iii) the one or more co-stimulation molecules do not comprise 41BB; (iv) the FAS agonist comprises a Fas ligand (FasL); or (v) the cell further comprises one or more of: (a) an exogenous polynucleotide encoding a chimeric antigen receptor (CAR); (b) an exogenous polynucleotide encoding a CD16 or a variant thereof, (c) CD38 knockout; and (d) an exogenous polynucleotide encoding a cytokine signaling complex comprising a partial or full peptide of a cell surface expressed exogenous cytokine and/or a receptor thereof. In some embodiments, the transmembrane region of the Fas redirector comprises: (i) a full length or at least a portion of the native or modified transmembrane region of FAS, CD2, CD36, CD3R, CD3γ, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide; or (ii) a full or partial length of the transmembrane domain of FAS.

In some embodiments of the cell or population thereof, (i) the extracellular binding domain of the Fas redirector comprises a sequence having at least about 85%, about 90%, about 95%, about 96%, about 97%, about 98%, or about 99% identity to SEQ ID NO: 1; (ii) the cytoplasmic signaling domain of the Fas redirector comprises: (a) a full or partial length of the intracellular domain (ICD) of MyD88 and CD40, represented by SEQ ID NO: 2; or (b) a full or partial length of the intracellular domain (ICD) of CD27, represented by SEQ ID NO: 3; or (c) a full or partial length of the intracellular domain (ICD) of CD28, represented by SEQ ID NO: 4; or (d) a full or partial length of the intracellular domain (ICD) of OX40, represented by SEQ ID NO: 5; or (e) a full or partial length of the intracellular domain (ICD) of IL12Rβ2, represented by SEQ ID NO: 6; or (f) a full or partial length of the intracellular domain (ICD) of IL18R1, represented by SEQ ID NO: 7; or (g) a full or partial length of the intracellular domain (ICD) of IL21R, represented by SEQ ID NO: 8; or (iii) the Fas redirector comprises an amino acid sequence that has at least about 85%, about 90%, about 95%, about 96%, about 97%, about 98%, or about 99% identity to any one of SEQ ID NOs: 9-15.

In various embodiments of the cell or population thereof, the cell further comprises one or more of: (i) at least one of the genotypes listed in Table 1; (ii) HLA-I deficiency and/or HLA-II deficiency; (iii) introduction of HLA-G or non-cleavable HLA-G, or knockout of one or both of CD58 and CD54; (iv) deletion or disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or (v) introduction of at least one of HLA-E, 4-1BBL, CD3, CD4, CD8, CD47, CD113, CD131, CD137, CD80, PDL1, A_{2A}R, antigen-specific TCR, chimeric fusion receptor (CFR), Fc receptor, an antibody or functional variant or fragment thereof, a checkpoint inhibitor, an engager, and surface triggering receptor for coupling with bi- or multi-specific or universal engagers. In various embodiments of the cell or population thereof, the cell comprises HLA-I deficiency and/or HLA-II deficiency; and optionally, wherein the cell comprises an exogenous polynucleotide encoding HLA-G, HLA-E, or a variant thereof, and/or comprises deletion or disruption of one or both of CD54 and CD58. In some embodiments, the HLA-I deficiency comprises deletion or disruption of at least one of: B2M, TAP1, TAP2, and Tapasin; or the HLA-II deficiency comprises deletion or disruption of at least one of: CIITA, RFX5, RFXAP, and RFXANK.

In various embodiments of the cell or population thereof, the derivative cell: (a) comprises a derivative CD34+ cell, a derivative hematopoietic stem and progenitor cell, a derivative hematopoietic multipotent progenitor cell, a derivative T cell progenitor, a derivative NK cell progenitor, a derivative T cell, a derivative NKT cell, a derivative NK cell, a derivative B cell, or a derivative effector cell having one or more functional features that are not present in a counterpart primary T, NK, NKT, and/or B cell; or is an allogeneic effector cell, wherein the effector cell is a derivative NK cell or a derivative T cell having at least one of the following characteristics comprising: (i) improved persistency and/or survival; (ii) increased resistance to activated recipient immune cells; (iii) increased cytotoxicity; (iv) improved tumor penetration; (v) enhanced or acquired ADCC; (vi) enhanced ability in migrating, and/or activating or recruiting bystander immune cells, to tumor sites; (vii) enhanced ability to reduce tumor immunosuppression; (viii) improved ability in rescuing tumor antigen escape; and (ix) reduced apoptosis and/or fratricide, in comparison to its native counterpart cell obtained from peripheral blood, umbilical cord blood, or other donor tissues.

In some embodiments of the cell or population thereof where the cell comprises an exogenous polynucleotide encoding a CD16 or a variant thereof, the exogenous CD16 comprises at least one of: (a) a high affinity non-cleavable CD16 (hnCD16) or a variant thereof, (b) F176V and S197P in ectodomain domain of CD16; (c) a full or partial ectodomain originated from CD64; (d) a non-native (or non-CD16) transmembrane domain; (e) a non-native (or non-CD16) intracellular domain; (f) a non-native (or non-CD16) signaling domain; (g) a non-native stimulatory domain; and (h) transmembrane, signaling, and stimulatory domains that are not originated from CD16, and are originated from a same or different polypeptide. In some embodiments, (a) the non-native transmembrane domain is derived from a CD3δ, CD3ε, CD3γ, CD3ζ, CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor (TCR) polypeptide; (b) the non-native stimulatory domain is derived from a CD27, CD28, 4-1BB, OX40, ICOS, PD-1, LAG-3, 2B4, BTLA, DAP10, DAP12, CTLA-4, or NKG2D polypeptide; (c) the non-native signaling domain is derived from a CD3ζ, 2B4, DAP10, DAP12, DNAM1, CD137 (4-1BB), IL21, IL7, IL12, IL15, NKp30, NKp44, NKp46, NKG2C, or NKG2D polypeptide; or (d) the non-native transmembrane domain is derived from NKG2D, the non-native stimulatory domain is derived from 2B4, and the non-native signaling domain is derived from CD3ζ.

In some embodiments of the cell or population thereof where the cell comprises an exogenous polynucleotide encoding a chimeric antigen receptor (CAR), the CAR is: (i) T cell specific or NK cell specific; (ii) a bi-specific antigen binding CAR; (iii) a switchable CAR; (iv) a dimerized CAR; (v) a split CAR; (vi) a multi-chain CAR; (vii) an inducible CAR; (viii) co-expressed with a cytokine signaling complex comprising a partial or full peptide of a cell surface expressed exogenous cytokine and/or a receptor thereof, optionally in separate constructs or in a bi-cistronic construct; (ix) co-expressed with a checkpoint inhibitor, optionally in separate constructs or in a bi-cistronic construct; and/or (x) optionally inserted at: (1) a TRAC or a TRBC locus, and/or is driven by an endogenous promoter of TCR, and/or the TCR is knocked out by the CAR insertion; (2) a safe harbor locus; or (3) a gene locus intended for disruption. In some embodiments, the CAR is: (i) specific to at least one of CD19, BCMA, B7H3, CD20, CD22, CD38, CD52, CD79b, CD123, EGFR, EGP2/EpCAM, GD2, GPRC5D, HER2, KLK2, MICA/B, MR1, MSLN, Muc1, Muc16, NYESO1, VEGF-R2, PSMA and PDL1; and/or (ii) specific to any one of ADGRE2, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), Mucin 1 (Muc-1), Mucin 16 (Muc-16), Mesothelin (MSLN), NKCSI, NKG2D ligands, c-Met, cancer-testis antigen NYESO-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen.

In some embodiments of the cell or population thereof where the cell comprises an exogenous polynucleotide encoding a cytokine signaling complex, the cytokine signaling complex comprises: (a) a partial or full peptide of a cell surface expressed exogenous cytokine and/or receptor thereof comprising at least one of IL2, IL4, IL6, IL7, IL9, IL10, IL11, IL12, IL15, IL18, IL21, or respective receptor(s) thereof; or (b) at least one of: (i) co-expression of IL15 and IL15Rα with a self-cleaving peptide in-between; (ii) a fusion protein of IL15 and IL15Rα; (iii) an IL15/IL15Rα fusion protein with intracellular domain of IL15Rα truncated (TL15Δ); (iv) a fusion protein of IL15 and membrane bound Sushi domain of IL15Rα; (v) a fusion protein of IL15 and IL15Rβ; (vi) a fusion protein of IL15 and common receptor γC, wherein the common receptor γC is native or modified; and (vii) a homodimer of IL15Rβ; wherein any one of (b)(i)-(vii) can be co-expressed with a CAR in separate constructs or in a bi-cistronic construct; or (c) at least one of: (i) a fusion protein of IL7 and IL7Rα; (ii) a fusion protein of IL7 and common receptor γC, wherein the common receptor γC is native or modified; and (iii) a homodimer of IL7Rβ, wherein any one of (c)(i)-(iii) is optionally co-expressed with a CAR in separate constructs or in a bi-cistronic construct; and optionally, (d) is transiently expressed.

In various embodiments of the cell or population thereof, the derivative cell is a derivative NK or a derivative T cell, wherein the derivative NK cell is capable of recruiting and/or migrating T cells to tumor sites, and wherein the derivative NK cell or the derivative T cell is capable of reducing tumor immunosuppression in the presence of one or more checkpoint inhibitors. In some embodiments, the one or more checkpoint inhibitors are antagonists to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, retinoic acid receptor alpha (Rara), TLR3, VISTA, NKG2A/HLA-E, or inhibitory KIR. In some embodiments, the one or more checkpoint inhibitors comprise: (a) one or more of atezolizumab, avelumab, durvalumab, ipilimumab, IPH4102, IPH43, IPH33, lirimumab, monalizumab, nivolumab, pembrolizumab, and their derivatives or functional equivalents; or (b) at least one of atezolizumab, nivolumab, and pembrolizumab.

In various embodiments of the cell or population thereof, the cell comprises: (i) one or more exogenous polynucleotides integrated in one safe harbor locus or locus intended for disruption; or (ii) more than two exogenous polynucleotides integrated in different safe harbor loci or loci intended for disruption. In some embodiments, the safe harbor locus or loci comprises at least one of AAVS1, CCR5, ROSA26, collagen, HTRP, H11, GAPDH, TCR or RUNX1; or wherein the gene locus or loci intended for disruption comprises at least one of B2M, TAP1, TAP2, tapasin, NLRC5, CIITA, RFXANK, RFX5, RFXAP, TCRα or TCRβ constant region, NKG2A, NKG2D, CD38, CD25, CD69, CD71, CD44, CD58, CD54, CD56, CIS, CBL-B, SOCS2, PD1, CTLA4, LAG3, TIM3, or TIGIT.

In some embodiments of the cell or population thereof, the cytoplasmic signaling domain of the Fas redirector in the cell comprises at least one of: a full or partial length of the intracellular domain (ICD) of MyD88 and CD40, represented by SEQ ID NO: 2; or a full or partial length of the intracellular domain (ICD) of CD27, represented by SEQ ID NO: 3; or a full or partial length of the intracellular domain (ICD) of OX40, represented by SEQ ID NO: 5. In some other embodiments, the cell or population thereof comprising said cytoplasmic signaling domain further comprises an exogenous polynucleotide encoding a fusion protein of a partial or full peptide of IL7 and a partial or full peptide of IL7Rα.

In another aspect, the present invention provides a composition comprising the cell or population thereof as described herein. In various embodiments, the composition further comprises one or more therapeutic agents. In some embodiments, the one or more therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). In some embodiments where the therapeutic agent is a checkpoint inhibitor, (i) the checkpoint inhibitor comprises: (a) one or more antagonists to checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxp1, GARP, HVEM, IDO, EDO, TDO, LAIR-1, MICA/B, NR4A2, MAFB, OCT-2, retinoic acid receptor alpha (Rara), TLR3, VISTA, NKG2A/ILA-E, or inhibitory KIR; (b) one or more of atezolizumab, avelumab, durvalumab, ipilimumab, IPH4102, IPH43, IPH33, lirimumab, monalizumab, nivolumab, pembrolizumab, and their derivatives or functional equivalents; (c) at least one of atezolizumab, nivolumab, and pembrolizumab; or (ii) the therapeutic agents comprise one or more of venetoclax, azacitidine, and pomalidomide. In some embodiments where the therapeutic agent is an antibody, the antibody comprises: (a) an anti-CD20 antibody, an anti-HER2 antibody, an anti-CD52 antibody, an anti-EGFR antibody, an anti-CD123 antibody, an anti-GD2 antibody, or an anti-PDL1 antibody; or (b) one or more of rituximab, veltuzumab, ofatumumab, ublituximab, ocaratuzumab, obinutuzumab, trastuzumab, pertuzumab, alemtuzumab, cetuximab, dinutuximab, avelumab, daclizumab, basiliximab, M-A251, 2A3, BC69, 24204, 22722, 24212, MAB23591, FN50, 298614, AF2359, CY1G4, DF1513, bivatuzumab, RG7356, G44-26, 7G3, CSL362, elotuzumab, and their humanized or Fc modified variants or fragments and their functional equivalents and biosimilars thereof. In some embodiments where the therapeutic agent is an engager, the engager comprises: (i) a bispecific T cell engager (BiTE); (ii) a bispecific killer cell engager (BiKE); or (iii) a tri-specific killer cell engager (TriKE); or the engager comprises: (a) a first binding domain recognizing an extracellular portion of CD3, CD5, CD16, CD28, CD32, CD33, CD64, CD89, NKG2C, NKG2D, or any functional variants thereof of the cell or a by-stander immune effector cell; and (b) a second binding domain specific to an antigen comprising any one of: B7H3, BCMA, CD10, CD19, CD20, CD22, CD24, CD30, CD33, CD34, CD38, CD44, CD52, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, Muc1, Muc16, PDL1, PSMA, PAMA, P-cadherin, ROR1, or VEGF-R2.

In another aspect, the present invention provides for therapeutic use of the composition provided herein by introducing the composition to a subject in need of an adoptive cell therapy, wherein the subject has an autoimmune disorder, a hematological malignancy, a solid tumor, cancer, or a virus infection.

In another aspect, the present invention provides a master cell bank (MCB) comprising the clonal iPSC provided herein.

In another aspect, the present invention provides a method of manufacturing the derivative cell provided herein, wherein the derivative cell is an effector cell, and the method comprises: differentiating a genetically engineered iPSC, wherein the genetically engineered iPSC comprises the exogenous polynucleotide encoding a Fas redirector that redirects Fas signaling upon binding to a FAS agonist, thereby providing the effector cell with improved apoptosis resistance and/or exhaustion resistance. In some embodiments of the method of manufacturing, the Fas redirector comprises: (a) an extracellular Fas binding domain comprising a partial or full peptide of an extracellular domain (ECD) of a Fas receptor (FAS), or a variant or an allele thereof, and (b) a cytoplasmic signaling domain comprising a partial or full peptide of an intracellular domain (ICD) of one or more co-stimulation molecules; and wherein the genetically engineered iPSC is a single cell, a clonal cell, or a cell line cell. In some embodiments of the method of manufacturing, (i) the Fas redirector further comprises a transmembrane region comprising a transmembrane domain, or a portion thereof, of a transmembrane protein; (ii) the one or more co-stimulation molecules comprise CD27, CD28, CD40, MyD88, OX40, IL12Rβ2, IL18R1, IL21R, or a combination thereof, (iii) the one or more co-stimulation molecules do not comprise 41BB; (iv) the FAS agonist comprises a Fas ligand (FasL); or (v) the genetically engineered iPSC comprising the Fas redirector further comprises one or more of: (a) an exogenous polynucleotide encoding a chimeric antigen receptor (CAR); (b) an exogenous polynucleotide encoding a CD16 or a variant thereof, (c) CD38 knockout; and (d) an exogenous polynucleotide encoding a cytokine signaling complex comprising a partial or full peptide of a cell surface expressed exogenous cytokine and/or a receptor thereof.

In some embodiments of the method of manufacturing, the transmembrane region of the Fas redirector comprises: (i) a full length or at least a portion of the native or modified transmembrane region of FAS, CD2, CD3δ, CD3R, CD3γ, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide; or (ii) a full or partial length of the transmembrane domain of FAS. In some embodiments, (i) the Fas redirector further comprises a transmembrane region comprising a transmembrane domain, or a portion thereof, of a transmembrane protein; (ii) the one or more co-stimulation molecules comprise CD27, CD28, CD40, MyD88, OX40, IL12Rβ2, IL18R1, IL21R, or a combination thereof, (iii) the one or more co-stimulation molecules do not comprise 41BB; (iv) the FAS agonist comprises a Fas ligand (FasL); or (v) the genetically engineered iPSC comprising the Fas redirector further comprises one or more of: (a) an exogenous polynucleotide encoding a chimeric antigen receptor (CAR); (b) an exogenous polynucleotide encoding a CD16 or a variant thereof, (c) CD38 knockout; and (d) an exogenous polynucleotide encoding a cytokine signaling complex comprising a partial or full peptide of a cell surface expressed exogenous cytokine and/or a receptor thereof. In some embodiments, the transmembrane region of the Fas redirector comprises: (i) a full length or at least a portion of the native or modified transmembrane region of FAS, CD2, CD3δ, CD3ε, CD3γ, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide; or (ii) a full or partial length of the transmembrane domain of FAS. In some embodiments, (i) the extracellular binding domain of the Fas redirector comprises a sequence having at least about 85%, about 90%, about 95%, about 96%, about 97%, about 98%, or about 99% identity to SEQ ID NO: 1; (ii) the cytoplasmic signaling domain of the Fas redirector comprises: (a) a full or partial length of the intracellular domain (ICD) of MyD88 and CD40, represented by SEQ ID NO: 2; or (b) a full or partial length of the intracellular domain (ICD) of CD27, represented by SEQ ID NO: 3; or (c) a full or partial length of the intracellular domain (ICD) of CD28, represented by SEQ ID NO: 4; or (d) a full or partial length of the intracellular domain (ICD) of OX40, represented by SEQ ID NO: 5; or (e) a full or partial length of the intracellular domain (ICD) of IL12Rβ2, represented by SEQ ID NO: 6; or (f) a full or partial length of the intracellular domain (ICD) of IL18R1, represented by SEQ ID NO: 7; or (g) a full or partial length of the intracellular domain (ICD) of IL21R, represented by SEQ ID NO: 8; or (iii) the Fas redirector comprises an amino acid sequence that has at least about 85%, about 90%, about 95%, about 96%, about 97%, about 98%, or about 99% identity to any one of SEQ ID Nos: 9-15.

In various embodiments of the method of manufacturing, the genetically engineered iPSC further comprises one or more edits resulting in: (i) at least one of the genotypes listed in Table 1; (ii) HLA-I deficiency and/or HLA-II deficiency; (iii) introduction of HLA-G or non-cleavable HLA-G, or knockout of one or both of CD58 and CD54; (iv) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; and/or (v) introduction of at least one of HLA-E, 4-1BBL, CD3, CD4, CD8, CD47, CD113, CD131, CD137, CD80, PDL1, A_{2A}R, antigen-specific TCR, chimeric fusion receptor (CFR), Fc receptor, an antibody or functional variant or fragment thereof, a checkpoint inhibitor, an engager, and surface triggering receptor for coupling with bi- or multi-specific or universal engagers. In some embodiments, the cell comprises HLA-I deficiency, and/or HLA-II deficiency; and optionally, wherein the cell comprises an exogenous polynucleotide encoding HLA-G, HLA-E, or a variant thereof, or comprises deletion or disruption of one or both of CD54 and CD58.

In various embodiments of the method of manufacturing, the method further comprises: genomically engineering an iPSC to knock in: (a) the polynucleotide encoding the signaling redirector receptor; and optionally, (b) the exogenous polynucleotide encoding the chimeric antigen receptor (CAR); and optionally (c) the exogenous polynucleotide encoding the CD16 or a variant thereof, and optionally further comprising genomically engineering the iPSC: (i) to knock out CD38, (ii) to knock out one or both of B2M and CIITA, (iii) to knock out one or both of CD58 and CD54, and/or (iv) to introduce HLA-G or non-cleavable HLA-G and/or the signaling complex comprising the partial or full peptide of the cell surface expressed exogenous cytokine and/or receptor thereof. In some embodiments, the genomic engineering comprises targeted editing. In some embodiments, the targeted editing comprises deletion, insertion, or in/del, and wherein the targeted editing is carried out by CRISPR, ZFN, TALEN, homing nuclease, homology recombination, or any other functional variation of these methods.

In another aspect, the present invention provides a method of improving effector cell durability or preventing cell death of effector cells in an adoptive cell therapy provided to a subject in need thereof by obtaining the effector cells as provided herein.

In another aspect, the present invention provides a method of improving efficacy of an adoptive cell therapy provided to a subject in need thereof, the method comprising administering the effector cells to the subject, wherein the effector cells comprise the derivative cell or population thereof as provided herein. In some embodiments, the method further comprises administering one or more therapeutic agents to the subject. In some embodiments, the one or more therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). In some embodiments where the therapeutic agent is a checkpoint inhibitor, (i) the checkpoint inhibitor comprises: (a) one or more antagonists to checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxp1, GARP, HVEM, IDO, EDO, TDO, LAIR-1, MICA/B, NR4A2, MAFB, OCT-2, retinoic acid receptor alpha (Rara), TLR3, VISTA, NKG2A/HLA-E, or inhibitory KIR; (b) one or more of atezolizumab, avelumab, durvalumab, ipilimumab, IPH4102, IPH43, IPH33, lirimumab, monalizumab, nivolumab, pembrolizumab, and their derivatives or functional equivalents; (c) at least one of atezolizumab, nivolumab, and pembrolizumab; or (ii) the therapeutic agents comprise one or more of venetoclax, azacitidine, and pomalidomide. In some embodiments where the therapeutic agent is an antibody, the antibody comprises: (a) an anti-CD20 antibody, an anti-HER2 antibody, an anti-CD52 antibody, an anti-EGFR antibody, an anti-CD123 antibody, an anti-GD2 antibody, or an anti-PDL1 antibody; or (b) one or more of rituximab, veltuzumab, ofatumumab, ublituximab, ocaratuzumab, obinutuzumab, trastuzumab, pertuzumab, alemtuzumab, cetuximab, dinutuximab, avelumab, daclizumab, basiliximab, M-A251, 2A3, BC69, 24204, 22722, 24212, MAB23591, FN50, 298614, AF2359, CY1G4, DF1513, bivatuzumab, RG7356, G44-26, 7G3, CSL362, elotuzumab, and their humanized or Fc modified variants or fragments and their functional equivalents and biosimilars thereof. In some embodiments where the therapeutic agent is an engager, the engager comprises: (i) a bispecific T cell engager (BiTE); (ii) a bispecific killer cell engager (BiKE); or (iii) a tri-specific killer cell engager (TriKE); or the engager comprises: (a) a first binding domain recognizing an extracellular portion of CD3, CD5, CD16, CD28, CD32, CD33, CD64, CD89, NKG2C, NKG2D, or any functional variants thereof of the cell or a by-stander immune effector cell; and (b) a second binding domain specific to an antigen comprising any one of: B7H3, BCMA, CD10, CD19, CD20, CD22, CD24, CD30, CD33, CD34, CD38, CD44, CD52, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, Muc1, Muc16, PDL1, PSMA, PAMA, P-cadherin, ROR1, or VEGF-R2.

In another aspect, the present invention provides a method of treating a subject in need of an adoptive cell therapy, wherein the method comprises infusing the subject with effector cells, wherein the effector cells comprise the derivative cell or population thereof provided herein in combination with an exogenous cytokine.

Various objects and advantages of the compositions and methods as provided herein will become apparent from the following description taken in conjunction with the accompanying drawings wherein are set forth, by way of illustration and example, certain embodiments of this invention.

### BRIEF DESCRIPTION OF THE DRAWINGS

FIGS. 1A-1D show that iPSC-derived CAR T cells can be transduced with Fas redirection constructs and expand in a scalable manufacturing process.

FIGS. 2A and 2B show Fas MFI expression (FIG. 2A), and percent positive of Thy1.2 (FIG. 2B) on transduced iPSC-derived CAR T cells throughout T cell differentiation.

FIGS. 3A and 3B show that Fas redirection constructs enhance CAR iT killing of engineered SKOV3 that express a CAR-specific antigen. Error bars represent the standard error of the mean; N=6.

FIG. 4 shows that Fas redirection constructs enhance CAR iT cell expansion and persistence upon stimulation against engineered SKOV3 that express a CAR-specific antigen. Error bars represent the standard error of the mean; N=6.

FIG. 5 shows that FAS redirection constructs enhance CAR iT cell killing of NALM6 target cells. Error bars represent the standard error of the mean; N=6.

FIG. 6 shows that Fas redirection constructs enhance CAR iT cell expansion and persistence upon stimulation against NALM6 target cells.

FIG. 7 shows that Fas redirection constructs provide protection to CAR iT cells against FAS agonist-induced apoptosis.

FIG. 8 shows that Fas redirection constructs can prompt downstream NF-κB signaling.

FIG. 9 shows a table with sample list for bulk RNAseq analysis.

FIGS. 10A-10D show illustrative experimental results for differential gene expression analysis comparing untransduced vs. Fas redirector iT cells.

FIGS. 11A-11D show illustrative experimental results for differential gene expression analysis comparing CAR-iT cells expressing Fas dominant negative vs. Fas redirector constructs.

FIGS. 12A and 12 B provide illustrative experimental results showing Fas-MyD88/CD40 expressing CD19-CAR_IL7RF exhibit enhanced long-term killing against engineered SKOV3 that express CD19.

FIGS. 13A and 13B provide illustrative experimental results showing Fas-MyD88/CD40 expressing CD19-CAR_IL7RF exhibit enhanced persistence against engineered SKOV3 that express CD19.

FIG. 14 provides illustrative experimental results showing Fas-MYD88/CD40 enhances tumor clearance against a SKOV3.CD19 tumor model.

FIG. 15 provides illustrative experimental results showing Fas-MyD88/CD40 enhances CAR persistence and T cell activation in the tumor microenvironment.

### DETAILED DESCRIPTION OF THE INVENTION

Genomic modification of iPSCs (induced pluripotent stem cells) includes polynucleotide insertion, deletion and substitution. Exogenous gene expression in genome-engineered iPSCs often encounters problems such as gene silencing or reduced gene expression after prolonged clonal expansion of the original genome-engineered iPSCs, after cell differentiation, and in dedifferentiated cell types from the cells derived from the genome-engineered iPSCs. On the other hand, direct engineering of primary immune cells such as T or NK cells is challenging, and presents a hurdle to the preparation and delivery of engineered immune cells for adoptive cell therapy. In various embodiments, the present invention provides an efficient, reliable, and targeted approach for stably integrating one or more exogenous genes, including suicide genes and other functional modalities, which provide improved therapeutic properties relating to engraftment, trafficking, homing, migration, cytotoxicity, viability, maintenance, expansion, longevity, self-renewal, persistence, and/or survival, into iPSC derivative cells, including but not limited to HSCs (hematopoietic stem and progenitor cells), T cell progenitor cells, NK cell progenitor cells, T cells, NKT cells, and NK cells.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% of a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition at a functionally inert, low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

As used herein, the terms “reprogramming” or “dedifferentiation” or “increasing cell potency” or “increasing developmental potency” refer to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or “iPSCs”, refers to stem cells that are produced in vitro, using reprogramming factor and/or small molecule chemical driven methods, from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta (i.e., are not totipotent).

As used herein, the term “multipotent stem cell” refers to a cell that has the developmental potential to differentiate into cells of one or more germ layers (ectoderm, mesoderm and endoderm), but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

Two types of pluripotency have previously been described: the “primed” or “metastable” state of pluripotency akin to the epiblast stem cells (EpiSC) of the late blastocyst, and the “naïve” or “ground” state of pluripotency akin to the inner cell mass of the early/preimplantation blastocyst. While both pluripotent states exhibit the characteristics as described above, the naïve or ground state further exhibits: (i) pre-inactivation or reactivation of the X-chromosome in female cells; (ii) improved clonality and survival during single-cell culturing; (iii) global reduction in DNA methylation; (iv) reduction of H3K27me3 repressive chromatin mark deposition on developmental regulatory gene promoters; and (v) reduced expression of differentiation markers relative to primed state pluripotent cells. Standard methodologies of cellular reprogramming in which exogenous pluripotency genes are introduced to a somatic cell, expressed, and then either silenced or removed from the resulting pluripotent cells are generally seen to have characteristics of the primed state of pluripotency. Under standard pluripotent cell culture conditions such cells remain in the primed state unless the exogenous transgene expression is maintained, wherein characteristics of the ground state are observed.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical inter-cell spacing.

As used herein, the term “subject” refers to any animal, preferably a human patient, livestock, or other domesticated animal.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate” or “maintain” refers to the sustaining, propagating (growing) and/or differentiating of cells outside of tissue or the body, for example in a sterile plastic (or coated plastic) cell culture dish or flask. “Cultivation” or “maintaining” may utilize a culture medium as a source of nutrients, hormones and/or other factors helpful to propagate and/or sustain the cells.

As used herein, the term “mesoderm” refers to one of the three germinal layers that appears during early embryogenesis and which gives rise to various specialized cell types including blood cells of the circulatory system, muscles, the heart, the dermis, skeleton, and other supportive and connective tissues.

As used herein, the term “definitive hemogenic endothelium” (HE) or “pluripotent stem cell-derived definitive hemogenic endothelium” (iHE) refers to a subset of endothelial cells that give rise to hematopoietic stem and progenitor cells in a process called endothelial-to-hematopoietic transition. The development of hematopoietic cells in the embryo proceeds sequentially from lateral plate mesoderm through the hemangioblast to the definitive hemogenic endothelium and hematopoietic progenitors.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34⁺ hematopoietic cells capable of giving rise to both mature myeloid and lymphoid cell types including T lineage cells, NK lineage cells and B lineage cells. Hematopoietic cells also include various subsets of primitive hematopoietic cells that give rise to primitive erythrocytes, megakarocytes and macrophages.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells in an MHC class I-restricted manner. A T cell can be any T cell, such as a cultured T cell, e.g., a primary T cell, or a T cell from a cultured T cell line, e.g., Jurkat, SupT1, etc., or a T cell obtained from a mammal. The T cell can be a CD3⁺ cell. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (γδ T cells), and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The term “T cell” can also refer to a genetically engineered T cell, such as a T cell modified to express a T cell receptor (TCR) or a chimeric antigen receptor (CAR). A T cell or T cell like effector cell can also be differentiated from a stem cell or progenitor cell (“a derived T cell” or “a derived T cell like effector cell”, or collectively, “a derivative T lineage cell”). A derived T cell like effector cell may have a T cell lineage in some respects, but at the same time has one or more functional features that are not present in a primary T cell. In this application, a T cell, a T cell like effector cell, a derived T cell, a derived T cell like effector cell, or a derivative T lineage cell, are collectively termed as “a T lineage cell”.

“CD4⁺ T cells” refers to a subset of T cells that express CD4 on their surface and are associated with cell-mediated immune response. They are characterized by secretion profiles following stimulation, which may include secretion of cytokines such as IFN-gamma, TNF-alpha, IL2, IL4 and IL10. “CD4” molecules are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8⁺ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refer to a subset of peripheral blood lymphocytes defined by the expression of CD56 or CD16 and the absence of the T cell receptor (CD3). An NK cell can be any NK cell, such as a cultured NK cell, e.g., a primary NK cell, or an NK cell from a cultured or expanded NK cell or a cell-line NK cell, e.g., NK-92, or an NK cell obtained from a mammal that is healthy or with a disease condition. As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceRγ, and EAT-2. In some embodiments, isolated subpopulations of CD56⁺ NK cells comprise expression of CD16, NKG2C, CD57, NKG2D, NCR ligands, NKp30, NKp40, NKp46, activating and inhibitory KIRs, NKG2A and/or DNAM-1. CD56⁺ can be dim or bright expression. An NK cell, or an NK cell like effector cell may be differentiated from a stem cell or progenitor cell (“a derived NK cell” or “a derived NK cell like effector cell”, or collectively, “a derivative NK lineage cell”). A derivative NK cell like effector cell may have an NK cell lineage in some respects, but at the same time has one or more functional features that are not present in a primary NK cell. In this application, an NK cell, an NK cell like effector cell, a derived NK cell, a derived NK cell like effector cell, or a derivative NK lineage cell, are collectively termed as “an NK lineage cell”.

As used herein, the term “NKT cells” or “natural killer T cells” or “NKT lineage cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are recognized. Invariant or type I NKT cells express a very limited TCR repertoire—a canonical α-chain (Vα24-Jα18 in humans) associated with a limited spectrum of β chains (Vβ11 in humans). The second population of NKT cells, called non-classical or non-invariant type II NKT cells, display a more heterogeneous TCR αβ usage. Type I NKT cells are considered suitable for immunotherapy. Adaptive or invariant (type I) NKT cells can be identified by the expression of one or more of the following markers: TCR Va24-Ja18, Vb11, CD1d, CD3, CD4, CD8, aGalCer, CD161 and CD56.

The term “effector cell” generally is applied to certain cells in the immune system that carry out a specific activity in response to stimulation and/or activation, or to cells that effect a specific function upon activation. As used herein, the term “effector cell” includes, and in some contexts is interchangeable with, immune cells, “differentiated immune cells,” and primary or differentiated cells that are edited and/or modulated to carry out a specific activity in response to stimulation and/or activation. Non-limiting examples of effector cells include primary-sourced or iPSC-derived T cells, NK cells, NKT cells, B cells, macrophages, and neutrophils.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an “isolated cell” is partly or completely separated from at least one component, including other substances, cells or cell populations, as it is found in nature or as it is grown, stored or subsisted in non-naturally occurring environments. Specific examples of isolated cells include partially pure cell compositions, substantially pure cell compositions and cells cultured in a medium that is non-naturally occurring. Isolated cells may be obtained by separating the desired cells, or populations thereof, from other substances or cells in the environment, or by removing one or more other cell populations or subpopulations from the environment.

As used herein, the term “purify” or the like refers to increasing purity. For example, the purity can be increased to at least 50%, 60%, 70%, 80%, 90%, 95%, 99%, or 100%.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. Thus, the term “vector” comprises the construct to be delivered. A vector can be a linear or a circular molecule. A vector can be integrating or non-integrating. The major types of vectors include, but are not limited to, plasmids, episomal vectors, viral vectors, cosmids, and artificial chromosomes. Viral vectors include, but are not limited to, adenovirus vectors, adeno-associated virus vectors, retrovirus vectors, lentivirus vectors, Sendai virus vectors, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” may further comprise replacement of the endogenous sequence or a nucleotide that is deleted with the one or more inserted nucleotides.

As used herein, the term “exogenous” is intended to mean that the referenced molecule or the referenced activity is introduced into, or is non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. “Polynucleotide” also refers to both double- and single-stranded molecules.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “subunit” as used herein refers to each separate polypeptide chain of a protein complex, where each separate polypeptide chain can form a stable folded structure by itself. Many protein molecules are composed of more than one subunit, where the amino acid sequences can either be identical for each subunit, or similar, or completely different. For example, CD3 complex is composed of CD3α, CD3ε, CD3δ, CD3γ, and CD3ζ subunits, which form the CD3ε/CD3γ, CD3ε/CD3δ, and CD3ζ/CD3ζ dimers. Within a single subunit, contiguous portions of the polypeptide chain frequently fold into compact, local, semi-independent units that are called “domains”. Many protein domains may further comprise independent “structural subunits”, also called subdomains, contributing to a common function of the domain. As such, the term “subdomain” as used herein refers to a protein domain inside of a larger domain, for example, a binding domain within an ectodomain of a cell surface receptor; or a stimulatory domain or a signaling domain of an endodomain of a cell surface receptor.

“Operably-linked” or “operatively linked,” interchangeable with “operably connected” or “operatively connected,” refers to the association of nucleic acid sequences on a single nucleic acid fragment (or amino acids in a polypeptide with multiple domains) so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation. As a further example, a receptor-binding domain can be operatively connected to an intracellular signaling domain, such that binding of the receptor to a ligand transduces a signal responsive to said binding.

“Fusion proteins” or “chimeric proteins”, as used herein, are proteins created through genetic engineering to join two or more partial or whole polynucleotide coding sequences encoding separate proteins, and the expression of these joined polynucleotides results in a single peptide or multiple polypeptides with functional properties derived from each of the original proteins or fragments thereof. Between two neighboring polypeptides of different sources in the fusion protein, a linker (or spacer) peptide can be added.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK cells or derivative T cells, as used throughout this application are cells differentiated from an iPSC, as compared to their primary counterpart obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSCs using genomic editing. In the aspect of a source cell obtained from a specifically selected donor, disease or treatment context, the genetic imprint contributing to preferential therapeutic attributes may include any context specific genetic or epigenetic modifications which manifest a retainable phenotype, i.e., a preferential therapeutic attribute, that is passed on to derivative cells of the selected source cell, irrespective of the underlying molecular events being identified or not. Donor-, disease-, or treatment response-specific source cells may comprise genetic imprints that are retainable in iPSCs and derived hematopoietic lineage cells, which genetic imprints include but are not limited to, prearranged monospecific TCR, for example, from a viral specific T cell or invariant natural killer T (iNKT) cell; trackable and desirable genetic polymorphisms, for example, homozygous for a point mutation that encodes for the high-affinity CD16 receptor in selected donors; and predetermined HLA requirements, i.e., selected HLA-matched donor cells exhibiting a haplotype with increased population. As used herein, preferential therapeutic attributes include improved engraftment, trafficking, homing, viability, self-renewal, persistence, immune response regulation and modulation, survival, and cytotoxicity of a derived cell. A preferential therapeutic attribute may also relate to antigen targeting receptor expression; HLA presentation or lack thereof; resistance to tumor microenvironment; induction of bystander immune cells and immune modulations; improved on-target specificity with reduced off-tumor effect; resistance to treatment such as chemotherapy. When derivative cells having one or more therapeutic attributes are obtained from differentiating an iPSC that has genetic imprint(s) conferring a preferential therapeutic attribute incorporated thereto, such derivative cells are also called “synthetic cells”. For example, synthetic effector cells, or synthetic NK cells or synthetic T cells, as used throughout this application are cells differentiated from a genomically modified iPSC, as compared to their primary counterpart obtained from natural/native sources such as peripheral blood, umbilical cord blood, or other donor tissues. In some embodiments, a synthetic cell possesses one or more non-native cell functions when compared to its closest counterpart primary cell.

The term “enhanced therapeutic property” as used herein, refers to a therapeutic property of a cell that is enhanced as compared to a typical immune cell of the same general cell type. For example, an NK cell with an “enhanced therapeutic property” will possess an enhanced, improved, and/or augmented therapeutic property as compared to a typical, unmodified, and/or naturally occurring NK cell. Therapeutic properties of an immune cell may include, but are not limited to, cell engraftment, trafficking, homing, viability, self-renewal, persistence, immune response regulation and modulation, survival, and cytotoxicity. Therapeutic properties of an immune cell are also manifested by antigen targeting receptor expression; HLA presentation or lack thereof; resistance to tumor microenvironment; induction of bystander immune cells and immune modulations; improved on-target specificity with reduced off-tumor effect; and/or resistance to treatment such as chemotherapy.

As used herein, the term “engager” refers to a molecule, e.g., a fusion polypeptide, which is capable of forming a link between an immune cell (e.g., a T cell, a NK cell, a NKT cell, a B cell, a macrophage, a neutrophil), and a tumor cell; and activating the immune cell. Examples of engagers include, but are not limited to, bi-specific T cell engagers (BiTEs), bi-specific killer cell engagers (BiKEs), tri-specific killer cell engagers (TriKEs), or multi-specific killer cell engagers, or universal engagers compatible with multiple immune cell types.

As used herein, the term “surface triggering receptor” refers to a receptor capable of triggering or initiating an immune response, e.g., a cytotoxic response. Surface triggering receptors may be engineered, and may be expressed on effector cells, e.g., a T cell, a NK cell, a NKT cell, a B cell, a macrophage, or a neutrophil. In some embodiments, the surface triggering receptor facilitates bi- or multi-specific antibody engagement between the effector cells and a specific target cell (e.g., a tumor cell) independent of the effector cells' natural receptors and cell types. Using this approach, one may generate iPSCs comprising a universal surface triggering receptor, and then differentiate such iPSCs into populations of various effector cell types that express the universal surface triggering receptor. By “universal”, it is meant that the surface triggering receptor can be expressed in, and activate, any effector cells irrespective of the cell type, and all effector cells expressing the universal receptor can be coupled or linked to the engagers recognizable by the surface triggering receptor, regardless of the engager's tumor binding specificities. In some embodiments, engagers having the same tumor targeting specificity are used to couple with the universal surface triggering receptor. In some embodiments, engagers having different tumor targeting specificity are used to couple with the universal surface triggering receptor. As such, one or multiple effector cell types can be engaged to kill one specific type of tumor cells in some cases, and to kill two or more types of tumors in other cases. A surface triggering receptor generally comprises a co-stimulatory domain for effector cell activation and an anti-epitope that is specific to the epitope of an engager. A bi-specific engager is specific to the anti-epitope of a surface triggering receptor on one end, and is specific to a tumor antigen on the other end.

As used herein, the term “safety switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch protein could mediate induction of apoptosis, inhibition of protein synthesis, DNA replication, growth arrest, transcriptional and post-transcriptional genetic regulation and/or antibody-mediated depletion. In some instance, the safety switch protein is activated by an exogenous molecule, e.g., a prodrug, that when activated, triggers apoptosis and/or cell death of a therapeutic cell. Examples of safety switch proteins include, but are not limited to, suicide genes such as caspase 9 (or caspase 3 or 7), thymidine kinase, cytosine deaminase, B cell CD20, modified EGFR, and any combination thereof. In this strategy, a prodrug that is administered in the event of an adverse event is activated by the suicide-gene product and kills the transduced cell.

As used herein, the term “pharmaceutically active proteins or peptides” refers to proteins or peptides that are capable of achieving a biological and/or pharmaceutical effect on an organism. A pharmaceutically active protein has healing, curative or palliative properties against a disease and may be administered to ameliorate, relieve, alleviate, reverse or lessen the severity of a disease. A pharmaceutically active protein also has prophylactic properties and is used to prevent the onset of a disease or to lessen the severity of such disease or pathological condition when it does emerge. “Pharmaceutically active proteins” include an entire protein or peptide or pharmaceutically active fragments thereof. The term also includes pharmaceutically active analogs of the protein or peptide or analogs of fragments of the protein or peptide. The term pharmaceutically active protein also refers to a plurality of proteins or peptides that act cooperatively or synergistically to provide a therapeutic benefit. Examples of pharmaceutically active proteins or peptides include, but are not limited to, receptors, binding proteins, transcription and translation factors, tumor growth suppressing proteins, antibodies or fragments thereof, growth factors, and/or cytokines.

As used herein, the term “signaling molecule” refers to any molecule that modulates, participates in, inhibits, activates, reduces, or increases, cellular signal transduction. “Signal transduction” refers to the transmission of a molecular signal in the form of chemical modification by recruitment of protein complexes along a pathway that ultimately triggers a biochemical event in the cell. Signal transduction pathways are well known in the art, and include, but are not limited to, G protein coupled receptor signaling, tyrosine kinase receptor signaling, integrin signaling, toll gate signaling, ligand-gated ion channel signaling, ERK/MAPK signaling pathway, Wnt signaling pathway, cAMP-dependent pathway, and IP3/DAG signaling pathway.

As used herein, the term “targeting modality” refers to a molecule, e.g., a polypeptide, that is genetically incorporated into a cell to promote antigen and/or epitope specificity that includes, but is not limited to, i) antigen specificity as it relates to a unique chimeric antigen receptor (CAR) or T cell receptor (TCR), ii) engager specificity as it relates to monoclonal antibodies or bispecific engagers, iii) targeting of transformed cells, iv) targeting of cancer stem cells, and v) other targeting strategies in the absence of a specific antigen or surface molecule.

As used herein, the term “specific” or “specificity” can be used to refer to the ability of a molecule, e.g., a receptor or an engager, to selectively bind to a target molecule, in contrast to non-specific or non-selective binding.

The term “adoptive cell therapy” as used herein refers to a cell-based immunotherapy that relates to the transfusion of autologous or allogeneic lymphocytes, whether the immune cells are isolated from a human donor or effector cells obtained from in vitro differentiation of a pluripotent cell; whether they are genetically modified or not; or whether they are primary donor cells or cells that have been passaged, expanded, or immortalized, ex vivo, after isolation from a donor.

As used herein, “lymphodepletion” and “lympho-conditioning” are used interchangeably to refer to the destruction of lymphocytes and T cells, typically prior to immunotherapy. The purpose of lympho-conditioning prior to the administration of an adoptive cell therapy is to promote homeostatic proliferation of effector cells as well as to eliminate regulatory immune cells and other competing elements of the immune system that compete for homeostatic cytokines. Thus, lympho-conditioning is typically accomplished by administering one or more chemotherapeutic agents to the subject prior to a first dose of the adoptive cell therapy. In various embodiments, lympho-conditioning precedes the first dose of the adoptive cell therapy by a few hours to a few days. Exemplary chemotherapeutic agents useful for lympho-conditioning include, but are not limited to, cyclophosphamide (CY), fludarabine (FLU), and those described below. However, a sufficient lymphodepletion through anti-CD38 mAb could provide an alternative conditioning process for the present iNK cell therapy, without or with minimal need of a CY/FLU-based lympho-conditioning procedure, as further described herein.

As used herein, “homing” or “trafficking” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of a cell to a target site. In some embodiments, a homing molecule is a chemokine receptor.

A “therapeutically sufficient amount”, as used herein, includes within its meaning a non-toxic, but sufficient and/or effective amount of a particular therapeutic agent and/or pharmaceutical composition to which it is referring to provide a desired therapeutic effect. The exact amount required will vary from subject to subject, depending on factors such as the patient's general health, the patient's age and the stage and severity of the condition being treated. In particular embodiments, a “therapeutically sufficient amount” is sufficient and/or effective to ameliorate, reduce, and/or improve at least one symptom associated with a disease or condition of the subject being treated.

Differentiation of pluripotent stem cells requires a change in the culture system, such as changing the stimuli agents in the culture medium or the physical state of the cells. The most conventional strategy utilizes the formation of embryoid bodies (EBs) as a common and critical intermediate to initiate lineage-specific differentiation. “Embryoid bodies” are three-dimensional clusters that have been shown to mimic embryo development as they give rise to numerous lineages within their three-dimensional area. Through the differentiation process, typically a few hours to days, simple EBs (for example, aggregated pluripotent stem cells elicited to differentiate) continue maturation and develop into a cystic EB at which time, typically days to a few weeks, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. As such, the pluripotent stem cell aggregates need to be transferred to differentiation medium that provides eliciting cues towards the lineage of choice. EB-based culture of pluripotent stem cells typically results in generation of differentiated cell populations (i.e., ectoderm, mesoderm and endoderm germ layers) with modest proliferation within the EB cell cluster. Although proven to facilitate cell differentiation, EBs, however, give rise to heterogeneous cells in variable differentiation states because of the inconsistent exposure of the cells in the three-dimensional structure to the differentiation cues within the environment. In addition, EBs are laborious to create and maintain. Moreover, cell differentiation through EB formation is accompanied with modest cell expansion, which also contributes to low differentiation efficiency.

In comparison, “aggregate formation,” as distinct from “EB formation,” can be used to expand the populations of pluripotent stem cell derived cells. For example, during aggregate-based pluripotent stem cell expansion, culture media are selected to maintain proliferation and pluripotency. Cell proliferation generally increases the size of the aggregates, forming larger aggregates, which can be mechanically or enzymatically dissociated into smaller aggregates to maintain cell proliferation within the culture and increase numbers of cells. As distinct from EB culture, cells cultured within aggregates in maintenance culture media maintain markers of pluripotency. The pluripotent stem cell aggregates require further differentiation cues to induce differentiation.

As used herein, “monolayer differentiation” is a term referring to a differentiation method distinct from differentiation through three-dimensional multilayered clusters of cells, i.e., “EB formation.” Monolayer differentiation, among other advantages disclosed herein, avoids the need for EB formation to initiate differentiation. Because monolayer culturing does not mimic embryo development such as is the case with EB formation, differentiation towards specific lineages is deemed to be minimal as compared to all three germ layer differentiation in EB formation.

As used herein, a “dissociated cell” or “single dissociated cell” refers to a cell that has been substantially separated or purified away from other cells or from a surface (e.g., a culture plate surface). For example, cells can be dissociated from an animal or tissue by mechanical or enzymatic methods. Alternatively, cells that aggregate in vitro can be enzymatically or mechanically dissociated from each other, such as by dissociation into a suspension of clusters, single cells or a mixture of single cells and clusters. In yet another alternative embodiment, adherent cells can be dissociated from a culture plate or other surface. Dissociation thus can involve breaking cell interactions with extracellular matrix (ECM) and substrates (e.g., culture surfaces), or breaking the ECM between cells.

As used herein, a “master cell bank” or “MCB” refers to a clonal master engineered iPSC line, which is a clonal population of iPSCs that have been engineered to comprise one or more therapeutic attributes, have been characterized, tested, qualified, and expanded, and have been shown to reliably serve as the starting cellular material for the production of cell-based therapeutics through directed differentiation in manufacturing settings. In various embodiments, an MCB is maintained, stored, and/or cryopreserved in multiple vessels to prevent genetic variation and/or potential contamination by reducing and/or eliminating the total number of times the iPS cell line is passaged, thawed or handled during the manufacturing processes.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of natural killer cells. The feeder cells may typically be inactivated when being co-cultured with other cells by irradiation or treatment with an anti-mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage, enhance proliferation capacity and promote maturation to a specialized cell type, such as an effector cell.

As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium. In some embodiments, a feeder-free environment is free of both feeder or stromal cells and is also not pre-conditioned by the cultivation of feeder cells.

“Functional” as used in the context of genomic editing or modification of iPSC, and derived non-pluripotent cells differentiated therefrom, or genomic editing or modification of non-pluripotent cells and derived iPSCs reprogrammed therefrom, refers to (1) at the gene level-successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level—successful removal, addition, or alteration of a cell function/characteristic via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at the iPSC, progenitor or dedifferentiated cellular origin.

“HLA deficient”, including HLA class I deficient, HLA class II deficient, or both, refers to cells that either lack, or no longer maintain, or have a reduced level of surface expression of a complete MHC complex comprising an HLA class I protein heterodimer and/or an HLA class II heterodimer, such that the diminished or reduced level is less than the level naturally detectable by other cells or by synthetic methods.

“Modified HLA deficient iPSC,” as used herein, refers to an HLA deficient iPSC that is further modified by introducing genes expressing proteins related, but not limited to improved differentiation potential, antigen targeting, antigen presentation, antibody recognition, persistence, immune evasion, resistance to suppression, proliferation, costimulation, cytokine stimulation, cytokine production (autocrine or paracrine), chemotaxis, and cellular cytotoxicity, such as non-classical HLA class I proteins (e.g., HLA-E and HLA-G), chimeric antigen receptor (CAR), T cell receptor (TCR), CD16 Fc Receptor, BCL11b, NOTCH, RUNX1, IL15, 4-1BB, DAP10, DAP12, CD24, CD3ζ, 4-1BBL, CD47, CD113, and PDL1. The cells that are “modified HLA deficient” also include cells other than iPSCs.

The term “ligand” refers to a substance that forms a complex with a target molecule to produce a signal by binding to a site on the target. The ligand may be a natural or artificial substance capable of specific binding to the target. The ligand may be in the form of a protein, a peptide, an antibody, an antibody complex, a conjugate, a nucleic acid, a lipid, a polysaccharide, a monosaccharide, a small molecule, a nanoparticle, an ion, a neurotransmitter, or any other molecular entity capable of specific binding to a target. The target to which the ligand binds, may be a protein, a nucleic acid, an antigen, a receptor, a protein complex, or a cell. A ligand that binds to and alters the function of the target and triggers a signaling response is called “agonistic” or “an agonist”. A ligand that binds to a target and blocks or reduces a signaling response is “antagonistic” or “an antagonist.”

The term “antibody” is used herein in the broadest sense and refers generally to an immune-response generating molecule that contains at least one binding site that specifically binds to a target, wherein the target may be an antigen, or a receptor that is capable of interacting with certain antibodies. For example, an NK cell can be activated by the binding of an antibody or the Fc region of an antibody to its Fc-gamma receptors (FcγR), thereby triggering the ADCC (antibody-dependent cellular cytotoxicity) mediated effector cell activation. A specific piece or portion of an antigen or receptor, or a target in general, to which an antibody binds is known as an epitope or an antigenic determinant. The term “antibody” includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. An antibody may be a murine antibody, a human antibody, a humanized antibody, a camel IgG, a single variable new antigen receptor (VNAR), a shark heavy-chain antibody (Ig-NAR), a chimeric antibody, a recombinant antibody, a single-domain antibody (dAb), an anti-idiotype antibody, a bi-specific-, multi-specific- or multimeric-antibody, or antibody fragment thereof. Anti-idiotype antibodies are specific for binding to an idiotope of another antibody, wherein the idiotope is an antigenic determinant of an antibody. A bi-specific antibody may be a BiTE (bi-specific T cell engager) or a BiKE (bi-specific killer cell engager), and a multi-specific antibody may be a TriKE (tri-specific Killer cell engager). Non-limiting examples of antibody fragments include Fab, Fab′, F(ab′)2, F(ab′)3, Fv, Fabc, pFc, Fd, single chain fragment variable (scFv), tandem scFv (scFv)2, single chain Fab (scFab), disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the antibody.

“Fc receptors,” abbreviated FcR, are classified based on the type of antibody that they recognize. For example, those that bind the most common class of antibody, IgG, are called Fc-gamma receptors (FcγR), those that bind IgA are called Fc-alpha receptors (FcαR) and those that bind IgE are called Fc-epsilon receptors (FcεR). The classes of FcRs are also distinguished by the cells that express them (macrophages, granulocytes, natural killer cells, T and B cells) and the signaling properties of each receptor. Fc-gamma receptors (FcγR) include several members, FcγRI (CD64), FcγRIIA (CD32), FcγRIIB (CD32), FcγRIIIA (CD16a), and FcγRIIIB (CD16b), which differ in their antibody affinities due to their different molecular structures.

“Chimeric Receptor” is a general term used to describe an engineered, artificial, or a hybrid receptor protein molecule that is made to comprise two or more portions of amino acid sequences that are originated from at least two different proteins. The chimeric receptor proteins have been engineered to give a cell the ability to initiate signal transduction and carry out downstream function upon binding of an agonistic ligand to the receptor. Exemplary “chimeric receptors” include, but are not limited to, chimeric antigen receptors (CARs), chimeric fusion receptors (CFRs), chimeric Fc receptors (CFcRs), as well as fusions of two or more receptors.

“Chimeric Fc Receptor,” abbreviated as CFcR, is a term used to describe engineered Fc receptors having their native transmembrane and/or intracellular signaling domains modified or replaced with non-native transmembrane and/or intracellular signaling domains. In some embodiments of the chimeric Fc receptor, in addition to having one of, or both of, the transmembrane and signaling domains being non-native, one or more stimulatory domains can be introduced to the intracellular portion of the engineered Fc receptor to enhance cell activation, expansion and function upon triggering of the receptor. Unlike a chimeric antigen receptor (CAR), which contains an antigen binding domain to a target antigen, the chimeric Fc receptor binds to an Fc fragment, or the Fc region of an antibody, or the Fc region comprised in an engager or a binding molecule and activates the cell function with or without bringing the targeted cell close in vicinity. For example, a Fcγ receptor can be engineered to comprise selected transmembrane, stimulatory, and/or signaling domains in the intracellular region that respond to the binding of IgG at the extracellular domain, thereby generating a CFcR. In one example, a CFcR is produced by engineering CD16, a Fcγ receptor, by replacing its transmembrane domain and/or intracellular domain. To further improve the binding affinity of the CD16-based CFcR, the extracellular domain of CD64 or the high-affinity variants of CD16 (F176V, for example) can be incorporated. In some embodiments of the CFcR where a high affinity CD16 extracellular domain is involved, the proteolytic cleavage site comprising a serine at position 197 is eliminated or is replaced such at the extracellular domain of the receptor is non-cleavable, i.e., not subject to shedding, thereby obtaining a hnCD16-based CFcR.

CD16, a FcγR receptor, has been identified to have two isoforms, Fc receptors FcγRIIIa (CD16a) and FcγRIIIb (CD16b). CD16a is a transmembrane protein expressed by NK cells, which binds monomeric IgG attached to target cells to activate NK cells and facilitate antibody-dependent cell-mediated cytotoxicity (ADCC). “High affinity CD16,” “non-cleavable CD16,” or “high affinity non-cleavable CD16” (abbreviated as hnCD16), as used herein, refers to a natural or non-natural variant of CD16. The wildtype CD16 has low affinity and is subject to ectodomain shedding, a proteolytic cleavage process that regulates the cells surface density of various cell surface molecules on leukocytes upon NK cell activation. F176V and F158V are exemplary CD16 polymorphic variants having high affinity. A CD16 variant having the cleavage site (position 195-198) in the membrane-proximal region (position 189-212) altered or eliminated is not subject to shedding. The cleavage site and the membrane-proximal region are described in detail in WO2015/148926, the complete disclosure of which is incorporated herein by reference. The CD16 S197P variant is an engineered non-cleavable version of CD16. A CD16 variant comprising both F158V and S197P has high affinity and is non-cleavable. Another exemplary high affinity and non-cleavable CD16 (hnCD16) variant is an engineered CD16 comprising an ectodomain originated from one or more of the 3 exons of the CD64 ectodomain.

“T cell receptor,” abbreviated as “TCR,” generally refers to a protein complex found on the surface of a T cell and is responsible for recognizing fragments of antigen peptides bound to major histocompatibility complex (MHC) molecules. Binding of a TCR to an antigen peptide initiates TCR-CD3 intracellular activation, recruitment of numerous signaling molecules, and branching and integrating signaling pathways, leading to mobilization of transcription factors that are important for gene expression and typical T cell growth and function acquisition. A typical TCR comprises two highly variable protein chains (a and p), with each chain comprising a constant region proximal to the cell membrane and a variable region (i.e., binding domain) that binds to the peptide/MHC.

I. Cells and Compositions Useful for Adoptive Cell Therapies with Enhanced Properties

Provided herein is a strategy to systematically engineer the regulatory circuitry of a clonal iPSC without impacting the differentiation potency and cell development biology of the iPSC and its derivative cells, while enhancing the therapeutic properties of the derivative cells differentiated from the iPSC. The iPSC-derived cells are functionally improved and suitable for adoptive cell therapies following a combination of selective modalities being introduced to the cells at the level of iPSC through genomic engineering. It was previously unclear whether altered iPSCs comprising one or more provided genetic edits still have the capacity to enter cell development, and/or to mature and generate functional differentiated cells while retaining modified activities and/or properties. Unanticipated failures during directed cell differentiation from iPSCs have been attributed to aspects including, but not limited to, development stage specific gene expression or lack thereof, requirements for HLA complex presentation, protein shedding of introduced surface expressing modalities, and the need for reconfiguration of differentiation protocols enabling phenotypic and/or functional change in the cell. The present application has shown that the one or more selected genomic modifications as provided herein does not negatively impact iPSC differentiation potency, and the functional effector cells derived from the engineered iPSC have enhanced and/or acquired therapeutic properties attributable to the individual or combined genomic modifications retained in the effector cells following the iPSC differentiation.

### 1. Exogenous FAS Signaling Redirector

Fas receptor (Fas) is a death receptor that when bound to its respective ligand, Fas ligand (FasL), can induce apoptosis. Multiple liquid and solid tumors, as well as activated peripheral B, T and NK cells express high levels of FasL. This elevated expression of FasL in a recipient represents a large hurdle to adoptive immune cell products. CAR-T and CAR-NK cell therapy products could be Fas positive either by canonical expression or through FasL-induced activation of Fas, thereby being targeted and killed through FasL-induced apoptosis in a tumor environment or through fratricide by neighboring product cells that express Fas ligand.

To provide genetic enhancements to adoptive cell products, a number of constructs encoding a Fas signaling redirector receptor (also termed as “Fas redirector” or “Fas signaling redirector”) were designed. The Fas signaling redirector receptor comprises an extracellular Fas binding domain comprising a partial or full peptide of an extracellular domain (ECD) of a Fas receptor, or a variant or an allele thereof, and a cytoplasmic signaling domain comprising a partial or full peptide of an intracellular domain (ICD) of one or more co-stimulation molecules, wherein the Fas signaling redirector receptor redirects Fas signaling upon FasL binding to provide the cell with improved apoptosis resistance and/or exhaustion resistance. The Fas redirector as described herein further comprises a transmembrane region comprising a transmembrane domain, or a portion thereof, of a transmembrane protein. In some embodiments, the transmembrane region and the extracellular binding domain of the Fas redirector are both from the Fas receptor. In some embodiments, the transmembrane region of the Fas redirector is from a transmembrane protein that is not a Fas receptor. In some embodiments, the transmembrane region and the cytoplasmic signaling domain of the Fas redirector are from a same protein or from different proteins.

In some embodiments, the extracellular binding domain of a Fas redirector comprises a sequence having at least about 85%, about 90%, about 95%, about 96%, about 97%, about 98%, or about 99% identity to SEQ ID NO: 1. In some embodiments, the extracellular binding domain of a Fas redirector comprises a sequence having at least about 90% identity to SEQ ID NO: 1. In some embodiments, the extracellular binding domain of a Fas redirector comprises a sequence having at least about 95% identity to SEQ ID NO: 1. In some embodiments, the extracellular binding domain of a Fas redirector comprises the amino acid sequence of SEQ ID NO: 1. As used herein and throughout the application, the percent identity between two sequences is a function of the number of identical positions shared by the sequences (i.e., % identity=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm recognized in the art.

SEQ ID NO: 1
QVTDINSKGLELRKTVTTVETQNLEGLHHDGQFCHKPCPPGERKARDCTV
NGDEPDCVPCQEGKEYTDKAHFSSKCRRCRLCDEGHGLEVEINCTRTQNT
KCRCKPNFFCNSTVCEHCDPCTKCEHGIIKECTLTSNTKCKEEGSRSN

In some embodiments, the transmembrane region of the Fas redirector comprises a full length or at least a portion of the native or modified transmembrane region of Fas, CD2, CD3δ, CD3δ, CD3γ, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide. In some embodiments, the transmembrane region of the Fas redirector is a full or partial length of the transmembrane domain of the Fas receptor.

In some embodiments, the co-stimulation molecule that provides the cytoplasmic signaling domain of the Fas redirector comprises CD27, CD28, CD40, MyD88, OX40, IL12Rβ2, IL18R1, IL21R, or combinations thereof. In some embodiments, the co-stimulation molecule that provides the cytoplasmic signaling domain of the Fas redirector is not 41BB by itself.

In some embodiments, the co-stimulation molecule that provides the cytoplasmic signaling domain of the Fas redirector comprises a full or partial length of the intracellular domain (ICD) of MyD88 and CD40, represented by SEQ ID NO: 2. In some embodiments, the co-stimulation molecule that provides the cytoplasmic signaling domain of the Fas redirector comprises a full or partial length of the intracellular domain (ICD) of CD27, represented by SEQ ID NO: 3. In some embodiments, the co-stimulation molecule that provides the cytoplasmic signaling domain of the Fas redirector comprises a full or partial length of the intracellular domain (ICD) of CD28, represented by SEQ ID NO: 4. In some embodiments, the co-stimulation molecule that provides the cytoplasmic signaling domain of the Fas redirector comprises a full or partial length of the intracellular domain (ICD) of OX40, represented by SEQ ID NO: 5. In some embodiments, the co-stimulation molecule that provides the cytoplasmic signaling domain of the Fas redirector comprises a full or partial length of the intracellular domain (ICD) of IL12Rβ2, represented by SEQ ID NO: 6. In some embodiments, the co-stimulation molecule that provides the cytoplasmic signaling domain of the Fas redirector comprises a full or partial length of the intracellular domain (ICD) of IL18R1, represented by SEQ ID NO: 7. In some embodiments, the co-stimulation molecule that provides the cytoplasmic signaling domain of the Fas redirector comprises a full or partial length of the intracellular domain (ICD) of IL21R, represented by SEQ ID NO: 8.

SEQ ID NO: 2
MAAGGPGAGSAAPVSSTSSLPLAALNMRVRRRLSLFLNVRTQVAADWTAL
AEEMDFEYLEIRQLETQADPTGRLLDAWQGRPGASVGRLLELLTKLGRDD
VLLELGPSIEEDCQKYILKQQQEEAEKPLQVAAVDSSVPRTAELAGITTL
DDPLGHMPKKVAKKPTNKAPHPKQEPQEINFPDDLPGSNTAAPVQETLHG
CQPVTQEDGKESRISVQERQ
SEQ ID NO: 3
QRRKYRSNKGESPVEPAEPCHYSCPREEEGSTIPIQEDYRKPEPACSP
SEQ ID NO: 4
RSKRSRLLHSDYMNMTPRRPGPTRKHYQPYAPPRDFAAYRS
SEQ ID NO: 5
ALYLLRRDQRLPPDAHKPPGGGSFRTPIQEEQADAHSTLAKI
SEQ ID NO: 6
SDPKPENPACPWTVLPAGDLPTHDGYLPSNIDDLPSHEAPLADSLEELEP
Q
SEQ ID NO: 7
YRVDLVLFYRHLTRRDETLTDGKTYDAFVSYLKECRPENGEEHTFAVEIL
PRVLEKHFGYKLCIFERDVVPGGAVVDEIHSLIEKSRRLIIVLSKSYMSN
EVRYELESGLHEALVERKIKIILIEFTPVTDFTFLPQSLKLLKSHRVLKW
KADKSLSYNSRFWKNLLYLMPAKTVKPGRDEPEVLPVLSES
SEQ ID NO: 8
SLKTHPLWRLWKKIWAVPSPERFEMPLYKGCSGDFKKWVGAPFTGSSLEL
GPWSPEVPSTLEVYSCHPPRSPAKRLQLTELQEPAELVESDGVPKPSFWP
TAQNSGGSAYSEERDRPYGLVSIDTVTVLDAEGPCTWPCSCEDDGYPALD
LDAGLEPSPGLEDPLLDAGTTVLSCGCVSAGSPGLGGPLGSLLDRLKPPL
ADGEDWAGGLPWGGRSPGGVSESEAGSPLAGLDMDTFDSGFVGSDCSSPV
ECDFTSPGDEGPPRSYLRQWVVIPPPLSSPGPQAS

In some embodiments, the Fas redirector comprising an extracellular Fas binding domain and a cytoplasmic signaling domain comprising a partial or full peptide of the ICD of MyD88 and CD40 comprises an amino acid sequence that has at least about 85%, about 90%, about 95%, about 96%, about 97%, about 98%, or about 99% identity to SEQ ID NO: 9, with understanding by one skilled in the art that the signal peptide sequence and/or transmembrane domain sequence of which could vary in length or in sequence, or could be replaced with that of a different protein. In some embodiments, the Fas redirector comprises an amino acid sequence having at least about 90% identity to SEQ ID NO: 9. In some embodiments, the Fas redirector comprises an amino acid sequence having at least about 95% identity to SEQ ID NO: 9. In some embodiments, the Fas redirector comprises the amino acid sequence of SEQ ID NO: 9.

SEQ ID NO: 9
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVMAAGGPGAGS
AAPVSSTSSLPLAALNMRVRRRLSLFLNVRTQVAADWTALAEEMDFEYLE
IRQLETQADPTGRLLDAWQGRPGASVGRLLELLTKLGRDDVLLELGPSIE
EDCQKYILKQQQEEAEKPLQVAAVDSSVPRTAELAGITTLDDPLGHMPKK
VAKKPTNKAPHPKQEPQEINFPDDLPGSNTAAPVQETLHGCQPVTQEDGK
ESRISVQERQ
signal peptide-FAS (ECD) - TM - MyD88 (ICD) -
CD40 (ICD)

In some embodiments, the Fas redirector comprising an extracellular Fas binding domain and a cytoplasmic signaling domain comprising a partial or full peptide of the ICD of CD27 comprises an amino acid sequence that has at least about 85%, about 90%, about 95%, about 96%, about 97%, about 98%, or about 99% identity to SEQ ID NO: 10, with understanding by one skilled in the art that the signal peptide sequence and/or transmembrane domain sequence of which could vary in length or in sequence, or could be replaced with that of a different protein. In some embodiments, the Fas redirector comprises an amino acid sequence having at least about 90% identity to SEQ ID NO: 10. In some embodiments, the Fas redirector comprises an amino acid sequence having at least about 95% identity to SEQ ID NO: 10. In some embodiments, the Fas redirector comprises the amino acid sequence of SEQ ID NO: 10.

SEQ ID NO: 10
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVQRRKYRSNKG
ESPVEPAEPCHYSCPREEEGSTIPIQEDYRKPEPACSP
signal peptide-FAS (ECD) - TM - CD27 (ICD)

In some embodiments, the Fas redirector comprising an extracellular Fas binding domain and a cytoplasmic signaling domain comprising a partial or full peptide of the ICD of CD28 comprises an amino acid sequence that has at least about 85%, about 90%, about 95%, about 96%, about 97%, about 98%, or about 99% identity to SEQ ID NO: 11, with understanding by one skilled in the art that the signal peptide sequence and/or transmembrane domain sequence of which could vary in length or in sequence, or could be replaced with that of a different protein. In some embodiments, the Fas redirector comprises an amino acid sequence having at least about 90% identity to SEQ ID NO: 11. In some embodiments, the Fas redirector comprises an amino acid sequence having at least about 95% identity to SEQ ID NO: 11. In some embodiments, the Fas redirector comprises the amino acid sequence of SEQ ID NO: 11.

SEQ ID NO: 11
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVRSKRSRLLHS
DYMNMTPRRPGPTRKHYQPYAPPRDFAAYRS
signal peptide-FAS (ECD) - TM - CD28 (ICD)

In some embodiments, the Fas redirector comprising an extracellular Fas binding domain and a cytoplasmic signaling domain comprising a partial or full peptide of the ICD of OX40 comprises an amino acid sequence that has at least about 85%, about 90%, about 95%, about 96%, about 97%, about 98%, or about 99% identity to SEQ ID NO: 12, with understanding by one skilled in the art that the signal peptide sequence and/or transmembrane domain sequence of which could vary in length or in sequence, or could be replaced with that of a different protein. In some embodiments, the Fas redirector comprises an amino acid sequence having at least about 90% identity to SEQ ID NO: 12. In some embodiments, the Fas redirector comprises an amino acid sequence having at least about 95% identity to SEQ ID NO: 12. In some embodiments, the Fas redirector comprises the amino acid sequence of SEQ ID NO: 12.

SEQ ID NO: 12
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVALYLLRRDQR
LPPDAHKPPGGGSFRTPIQEEQADAHSTLAKI
signal peptide-FAS (ECD) - TM - OX40 (ICD)

In some embodiments, the Fas redirector comprising an extracellular Fas binding domain and a cytoplasmic signaling domain comprising a partial or full peptide of the ICD of IL12Rβ2 comprises an amino acid sequence that has at least about 85%, about 90%, about 95%, about 96%, about 97%, about 98%, or about 99% identity to SEQ ID NO: 13, with understanding by one skilled in the art that the signal peptide sequence and/or transmembrane domain sequence of which could vary in length or in sequence, or could be replaced with that of a different protein. In some embodiments, the Fas redirector comprises an amino acid sequence having at least about 90% identity to SEQ ID NO: 13. In some embodiments, the Fas redirector comprises an amino acid sequence having at least about 95% identity to SEQ ID NO: 13. In some embodiments, the Fas redirector comprises the amino acid sequence of SEQ ID NO: 13.

SEQ ID NO: 13
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVSDPKPENPAC
PWTVLPAGDLPTHDGYLPSNIDDLPSHEAPLADSLEELEPQ
signal peptide-FAS (ECD) - TM - IL12Rb2 (ICD)

In some embodiments, the Fas redirector comprising an extracellular Fas binding domain and a cytoplasmic signaling domain comprising a partial or full peptide of the ICD of IL18R1 comprises an amino acid sequence that has at least about 85%, about 90%, about 95%, about 96%, about 97%, about 98%, or about 99% identity to SEQ ID NO: 14, with understanding by one skilled in the art that the signal peptide sequence and/or transmembrane domain sequence of which could vary in length or in sequence, or could be replaced with that of a different protein. In some embodiments, the Fas redirector comprises an amino acid sequence having at least about 90% identity to SEQ ID NO: 14. In some embodiments, the Fas redirector comprises an amino acid sequence having at least about 95% identity to SEQ ID NO: 14. In some embodiments, the Fas redirector comprises the amino acid sequence of SEQ ID NO: 14.

SEQ ID NO: 14
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVYRVDLVLFYR
HLTRRDETLTDGKTYDAFVSYLKECRPENGEEHTFAVEILPRVLEKHFGY
KLCIFERDVVPGGAVVDEIHSLIEKSRRLIIVLSKSYMSNEVRYELESGL
HEALVERKIKIILIEFTPVTDFTFLPQSLKLLKSHRVLKWKADKSLSYNS
RFWKNLLYLMPAKTVKPGRDEPEVLPVLSES
signal peptide-FAS (ECD) - TM - IL18R1 (ICD)

In some embodiments, the Fas redirector comprising an extracellular Fas binding domain and a cytoplasmic signaling domain comprising a partial or full peptide of the ICD of IL21R comprises an amino acid sequence that has at least about 85%, about 90%, about 95%, about 96%, about 97%, about 98%, or about 99% identity to SEQ ID NO: 15, with understanding by one skilled in the art that the signal peptide sequence and/or transmembrane domain sequence of which could vary in length or in sequence, or could be replaced with that of a different protein. In some embodiments, the Fas redirector comprises an amino acid sequence having at least about 90% identity to SEQ ID NO: 15. In some embodiments, the Fas redirector comprises an amino acid sequence having at least about 95% identity to SEQ ID NO: 15. In some embodiments, the Fas redirector comprises the amino acid sequence of SEQ ID NO: 15.

SEQ ID NO: 15
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVSLKTHPLWRL
WKKIWAVPSPERFFMPLYKGCSGDFKKWVGAPFTGSSLELGPWSPEVPST
LEVYSCHPPRSPAKRLQLTELQEPAELVESDGVPKPSFWPTAQNSGGSAY
SEERDRPYGLVSIDTVTVLDAEGPCTWPCSCEDDGYPALDLDAGLEPSPG
LEDPLLDAGTTVLSCGCVSAGSPGLGGPLGSLLDRLKPPLADGEDWAGGL
PWGGRSPGGVSESEAGSPLAGLDMDTFDSGFVGSDCSSPVECDFTSPGDE
GPPRSYLRQWVVIPPPLSSPGPQAS
signal peptide-FAS (ECD) - TM - IL21R (ICD)

As such, in various embodiments, a Fas redirector as provided herein may be introduced to an induced pluripotent cell (iPSC) using one or more of the construct designs described above, and to its derivative cells upon iPSC differentiation. In addition to an iPSC, a clonal iPSC, a clonal iPS cell line, and iPSC-derived effector cells comprising at least one engineered modality as disclosed herein are provided. Also provided is a master cell bank comprising single cell sorted and expanded clonal engineered iPSCs having at least an exogenously introduced Fas redirector as described in this section, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

Accordingly, in some embodiments, the present invention provides iPSCs and derivative cells therefrom comprising an exogenous Fas redirector (“Fas” in Table 1), wherein the cells, including derivative T and NK cells, are useful for overcoming Fas induced apoptosis or fratricide, and wherein the Fas redirector comprises a partial or full peptide of an extracellular domain (ECD) of a Fas receptor, or a variant or an allele thereof; and a partial or full peptide of an intracellular domain (ICD) of one or more co-stimulation molecules. In some embodiments, the iPSC and derivative cells thereof comprise one or more additional genomic edits as described herein, without adversely impacting the differentiation potential of the iPSC and function of the derived effector cells including derivative T and NK cells.

### 2. Chimeric Antigen Receptor (CAR) Expression

Applicable to the genetically engineered iPSC and derivative effector cells thereof may be any CAR design known in the art. CAR is a fusion protein generally including an ectodomain that comprises an antigen recognition domain, a transmembrane domain, and an endodomain. In some embodiments, the ectodomain can further include a signal peptide or leader sequence and/or a spacer. In some embodiments, the endodomain can further comprise a signaling peptide that activates the effector cell expressing the CAR. In some embodiments, the endodomain can further comprise a signaling domain, where the signaling domain originates from a cytoplasmic domain of a signal transducing protein specific to T and/or NK cell activation or functioning. In some embodiments, the antigen recognition domain can specifically bind an antigen. In some embodiments, the antigen recognition domain can specifically bind an antigen associated with a disease or pathogen. In some embodiments, the disease-associated antigen is a tumor antigen, wherein the tumor may be a liquid or a solid tumor. In some embodiments, the CAR is suitable to activate either T or NK lineage cells expressing the CAR. In some embodiments, the CAR is NK cell-specific for comprising NK-specific signaling components. In certain embodiments, the T cells are derived from an iPSC comprising the CAR, and the derivative T lineage cells may comprise T helper cells, cytotoxic T cells, memory T cells, regulatory T cells, natural killer T cells, αβ T cells, γδ T cells, or a combination thereof. In certain embodiments, the NK cells are derived from iPSCs comprising the CAR.

In certain embodiments, the antigen recognition region/domain comprises a murine antibody, a human antibody, a humanized antibody, a camel Ig, a shark heavy-chain-only antibody (VNAR), Ig NAR, a chimeric antibody, a recombinant antibody, or an antibody fragment thereof. Non-limiting examples of antibody fragments include Fab, Fab′, F(ab′)2, F(ab′)3, Fv, single chain antigen binding fragment (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody. In some embodiments, the antigen recognition region of a CAR originates from the binding domain of a T cell receptor (TCR) that targets a tumor associated antigen (TAA).

Non-limiting examples of antigens that may be targeted by a CAR include ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NYESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and various pathogen antigens known in the art. Non-limiting examples of pathogens include viruses, bacteria, fungi, parasites, and protozoa capable of causing diseases.

Thus, in some embodiments, the genetically engineered iPSC and its derivative cells comprise an exogenous polynucleotide encoding a CAR, where the CAR is specific to a target including, but not limited to, B7H3, CD19, BCMA, CD20, CD22, CD38, CD52, CD79b, CD123, EGFR, EGP2/EpCAM, GD2, GPRC5D, HER2, KLK2, MICA/B, MR1, MSLN, Muc1, Muc16, NYESO1, VEGF-R2, PSMA and PDL1.

In some embodiments, the transmembrane domain of a CAR comprises a full length or at least a portion of the native or modified transmembrane region of CD2, CD3δ, CD3δ, CD3γ, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide.

In some embodiments, the signal transducing peptide of the endodomain (or intracellular domain) comprises a full length or at least a portion of a polypeptide of 2B4 (Natural killer Cell Receptor 2B4), 4-1BB (Tumor necrosis factor receptor superfamily member 9), CD16 (IgG Fc region Receptor III-A), CD2 (T-cell surface antigen CD2), CD28 (T-cell-specific surface glycoprotein CD28), CD28H (Transmembrane and immunoglobulin domain-containing protein 2), CD3ζ (T-cell surface glycoprotein CD3 zeta chain), CD3ζ1XX (CD3ζ variant), DAP10 (Hematopoietic cell signal transducer), DAP12 (TYRO protein tyrosine kinase-binding protein), DNAM1 (CD226 antigen), FcERIγ (High affinity immunoglobulin epsilon receptor subunit gamma), IL21R (Interleukin-21 receptor), IL-2Rβ/IL-15RB (Interleukin-2 receptor subunit beta), IL-2Rγ (Cytokine receptor common subunit gamma), IL-7R (Interleukin-7 receptor subunit alpha), KIR2DS2 (Killer cell immunoglobulin-like receptor 2DS2), NKG2D (NKG2-D type II integral membrane protein), NKp30 (Natural cytotoxicity triggering receptor 3), NKp44 (Natural cytotoxicity triggering receptor 2), NKp46 (Natural cytotoxicity triggering receptor 1), CS1(SLAM family member 7), and CD8 (T-cell surface glycoprotein CD8 alpha chain).

In some embodiments, the endodomain of a CAR further comprises a second signaling domain, and optionally a third signaling domain, where each of the first, second, and third signaling domains are different. In particular embodiments, the second and/or the third signaling domain comprises a cytoplasmic domain, or a portion thereof, of 2B4, 4-1BB, CD16, CD2, CD28, CD28H, CD3ζ, DAP10, DAP12, DNAM1, FcERIγ IL21R, IL-2Rβ (IL-15Rβ), IL-2Rγ, IL-7R, KIR2DS2, NKG2D, NKp30, NKp44, NKp46, CD3ζ1XX, CS1, or CD8.

In certain embodiments, the endodomain of a CAR further comprises at least one co-stimulatory signaling region. The co-stimulatory signaling region can comprise a full length or at least a portion of a polypeptide of CD27, CD28, 4-1BB, OX40, ICOS, PD-1, LAG-3, 2B4, BTLA, DAP10, DAP12, CTLA-4, or NKG2D, or any combination thereof.

In some embodiments, the CAR applicable to the cells provided herein comprises a co-stimulatory domain derived from CD28, and a signaling domain comprising the native or modified ITAM1 of CD3ζ, represented by an amino acid sequence having at least about 85%, about 90%, about 95%, about 96%, about 97%, about 98%, or about 99% identity to SEQ ID NO: 16. In some embodiments, the CAR comprises an amino acid sequence with at least about 90% identity to SEQ ID NO: 16. In some embodiments, the CAR comprises an amino acid sequence with at least about 95% identity to SEQ ID NO: 16. In some embodiments, the CAR comprises the amino acid sequence of SEQ ID NO: 16. In a further embodiment, the CAR comprising a co-stimulatory domain derived from CD28, and a native or modified ITAM1 of CD3ζ also comprises a hinge domain and trans-membrane domain derived from CD28, wherein an scFv may be connected to the trans-membrane domain through the hinge, and the CAR comprises an amino acid sequence of at least about 85%, about 90%, about 95%, about 96%, about 97%, about 98%, or about 99% identity to SEQ ID NO: 17. In some embodiments, the CAR comprises an amino acid sequence with at least about 90% identity to SEQ ID NO: 17. In some embodiments, the CAR comprises an amino acid sequence with at least about 95% identity to SEQ ID NO: 17. In some embodiments, the CAR comprises the amino acid sequence of SEQ ID NO: 17.

SEQ ID NO: 16
RSKRSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQEGLF
NELQKDKMAEAFSEIGMKGERRRGKGHDGLFQGLSTATKDTFDALHMQAL
PPR
(153 a.a. CD28 co-stim + CD3ζITAM)
SEQ ID NO: 17
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRSRVKFSRSADAPAYQQGQNQLYNELNLGRREEYDVLDKRRGRDP
EMGGKPRRKNPQEGLFNELQKDKMAEAFSEIGMKGERRRGKGHDGLFQGL
STATKDTFDALHMQALPPR
(219 a.a. CD28 hinge + CD28 TM + CD28 co-stim +
CD3ζITAM)

In various embodiments, the CAR applicable to the cells provided herein comprises a transmembrane domain derived from NKG2D, a co-stimulatory domain derived from 2B4, and a signaling domain comprising the native or modified CD3ζ, represented by an amino acid sequence of at least about 85%, about 90%, about 95%, about 96%, about 97%, about 98%, or about 99% identity to SEQ ID NO: 18. In some embodiments, the CAR comprises an amino acid sequence with at least about 90% identity to SEQ ID NO: 18. In some embodiments, the CAR comprises an amino acid sequence with at least about 95% identity to SEQ ID NO: 18. In some embodiments, the CAR comprises the amino acid sequence of SEQ ID NO: 18. The CAR comprising a transmembrane domain derived from NKG2D, a co-stimulatory domain derived from 2B4, and a signaling domain comprising the native or modified CD3ζ may further comprise a CD8 hinge, wherein the amino acid sequence of such a structure is of at least about 85%, about 90%, about 95%, about 96%, about 97%, about 98%, or about 99% identity to SEQ ID NO: 19. In some embodiments, the CAR comprises an amino acid sequence with at least about 90% identity to SEQ ID NO: 19. In some embodiments, the CAR comprises an amino acid sequence with at least about 95% identity to SEQ ID NO: 19. In some embodiments, the CAR comprises the amino acid sequence of SEQ ID NO: 19.

SEQ ID NO: 18
SNLFVASWIAVMIIFRIGMAVAIFCCFFFPSWRRKRKEKQSETSPKEFLT
IYEDVKDLKTRRNHEQEQTFPGGGSTIYSMIQSQSSAPTSQEPAYTLYSL
IQPSRKSGSRKRNHSPSFNSTIYEVIGKSQPKAQNPARLSRKELENFDVY
SRVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
(263 a.a NKG2D TM + 2B4 + CD3ζ)
SEQ ID NO: 19
TTTPAPRPPTPAPTIASQPLSLRPEACRPAAGGAVHTRGLDFACDSNLFV
ASWIAVMIIFRIGMAVAIFCCFFFPSWRRKRKEKQSETSPKEFLTIYEDV
KDLKTRRNHEQEQTFPGGGSTIYSMIQSQSSAPTSQEPAYTLYSLIQPSR
KSGSRKRNHSPSFNSTIYEVIGKSQPKAQNPARLSRKELENFDVYSRVKF
SRSADAPAYKQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPR
(308 a.a CD8 hinge + NKG2D TM + 2B4 + CD3ζ)

Non-limiting CAR strategies further include heterodimeric, conditionally activated CAR through dimerization of a pair of intracellular domain (see for example, U.S. Pat. No. 9,587,020); split CAR, where homologous recombination of antigen binding, hinge, and endo-domains to generate a CAR (see for example, U.S. Pub. No. 2017/0183407); multi-chain CAR that allows non-covalent link between two transmembrane domains connected to an antigen binding domain and a signaling domain, respectively (see for example, U.S. Pub. No. 2014/0134142); CARs having bispecific antigen binding domain (see for example, U.S. Pat. No. 9,447,194), or having a pair of antigen binding domains recognizing same or different antigens or epitopes (see for example, U.S. Pat. No. 8,409,577), or a tandem CAR (see for example, Hegde et al., J Clin Invest. 2016; 126(8):3036-3052); inducible CAR (see for example, U.S. Pub. Nos. 2016/0046700, 2016/0058857, and 2017/0166877); switchable CAR (see for example, U.S. Pub. No. 2014/0219975); and any other designs known in the art.

In a further embodiment, the iPSC and its derivative effector cells comprising a Fas redirector and optionally a CAR have the CAR inserted in a TCR constant region, leading to endogenous TCR knockout (TCR^{KO}), and optionally placing CAR expression under the control of the endogenous TCR promoter. In some other embodiments, the CAR inserted in the TCR constant region is specific to a tumor antigen comprising at least one of MR1, NYESO1, MICA/B, EpCAM, EGFR, B7H3, Muc1, Muc16, CD19, BCMA, CD20, CD22, CD38, CD123, HER2, CD52, GD2, MSLN, VEGF-R2, PSMA and PDL1. Additional CAR insertion sites include, but are not limited to, AAVS1, CCR5, ROSA26, collagen, HTRP, H11, GAPDH, RUNX1, B2M, TAP1, TAP2, tapasin, NLRC5, CIITA, RFXANK, RFX5, RFXAP, NKG2A, NKG2D, CD25, CD38, CD44, CD58, CD54, CD56, CD69, CD71, CIS, CBL-B, SOCS2, PD1, CTLA4, LAG3, TIM3, and TIGIT.

As such, aspects of the present invention provide genomically engineered iPSCs and derivative cells obtained from differentiating the genomically engineered iPSCs, wherein the iPSCs and the derivative cells comprise an exogenous polynucleotide encoding a Fas redirector and a CAR, optionally along with one or more additional modified modalities, as provided in Table 1, without adversely impacting the differentiation potential of the iPSC and function of the derived effector cells, including derivative T and NK cells. Also provided is a master cell bank comprising single cell sorted and expanded clonal engineered iPSCs having at least an exogenously introduced Fas redirector and optionally a CAR, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 3. CD16 Knock-In

CD16 has been identified as two isoforms, Fc receptors FcγRIIIa (CD16a; NM_000569.6) and FcγRIIIb (CD16b; NM_000570.4). CD16a is a transmembrane protein expressed by NK cells, which binds monomeric IgG attached to target cells to activate NK cells and facilitate antibody-dependent cell-mediated cytotoxicity (ADCC). CD16b is exclusively expressed by human neutrophils. “High affinity CD16,” “non-cleavable CD16,” or “high affinity non-cleavable CD16,” as used herein, refers to various CD16 variants. The wildtype CD16 has low affinity and is subject to ectodomain shedding, a proteolytic cleavage process that regulates cell surface density of various cell surface molecules on leukocytes upon NK cell activation. F176V (also called F158V in some publications) is an exemplary CD16 polymorphic variant having high affinity; whereas an S197P variant is an example of a genetically engineered non-cleavable version of CD16. An engineered CD16 variant comprising both F176V and S197P has high affinity and is non-cleavable, which was described in greater detail in International Pub. No. WO2015/148926, the complete disclosure of which is incorporated herein by reference. In addition, a chimeric CD16 receptor with the ectodomain of CD16 essentially replaced with at least a portion of CD64 ectodomain can also achieve the desired high affinity and non-cleavable features of a CD16 receptor capable of carrying out ADCC. In some embodiments, the replacement ectodomain of a chimeric CD16 comprises one or more of EC1, EC2, and EC3 exons of CD64 (UniPRotKB_P12314 or its isoform or polymorphic variant).

Unlike the endogenous CD16 expressed by primary NK cells which gets cleaved from the cellular surface following NK cell activation, the various non-cleavable versions of CD16 in derivative NK cells avoid CD16 shedding and maintain constant expression. In derivative NK cells, non-cleavable CD16 increases expression of TNFα and CD107a, indicative of improved cell functionality. Non-cleavable CD16 also enhances the antibody-dependent cell-mediated cytotoxicity (ADCC), and the engagement of bi-, tri-, or multi-specific engagers. ADCC is a mechanism of NK cell mediated lysis through the binding of CD16 to antibody-coated target cells. The additional high affinity characteristics of the introduced hnCD16 in a derived NK cell also enables in vitro loading of an ADCC antibody to the NK cell through hnCD16 before administering the cell to a subject in need of a cell therapy. As provided herein, in some embodiments, the hnCD16 may comprise F176V and S197P, or may comprise a full or partial length ectodomain originated from CD64, or may further comprise at least one of non-native transmembrane domain, stimulatory domain and signaling domain. As disclosed, the present application also provides a derivative NK cell or a cell population thereof, preloaded with one or more pre-selected ADCC antibodies in an amount sufficient for therapeutic use in a treatment of a condition, a disease, or an infection as further detailed below.

Unlike primary NK cells, mature T cells from a primary source (i.e., natural/native sources such as peripheral blood, umbilical cord blood, or other donor tissues) do not express CD16. It was unexpected that an iPSC comprising an expressed exogenous non-cleavable CD16 did not impair the T cell developmental biology and was able to differentiate into functional derivative T lineage cells that not only express the exogenous CD16, but also are capable of carrying out function through an acquired ADCC mechanism. This acquired ADCC in the derivative T lineage cell can additionally be used as an approach for dual targeting and/or to rescue antigen escape often occurred with CAR-T cell therapy, where the tumor relapses with reduced or lost CAR-T targeted antigen expression or expression of a mutated antigen to avoid recognition by the CAR (chimerical antigen receptor). When said derivative T lineage cell comprises acquired ADCC through exogenous CD16, including functional variants and CD16-based CFcR, expression, and when an antibody targets a different tumor antigen from the one targeted by the CAR, the antibody can be used to rescue CAR-T antigen escape and reduce or prevent relapse or recurrence of the targeted tumor often seen in CAR-T treatment. Such a strategy to reduce and/or prevent antigen escape while achieving dual targeting is equally applicable to NK cells expressing one or more CARs.

As such, various embodiments of an exogenous CD16 introduced to a cell include functional CD16 variants and chimeric receptors thereof. In some embodiments, the functional CD16 variant is a high-affinity non-cleavable CD16 receptor (hnCD16). An hnCD16, in some embodiments, comprises both F176V and S197P; and in some embodiments, comprises F176V and with the cleavage region eliminated. In some other embodiments, a hnCD16 comprises a sequence having an identity of at least 50%, 55%, 60%, 65%, 70%, 75%, 80%, 85%, 90%, 95%, 99%, 100%, or any percentage in-between, when compared to any of the exemplary sequences, SEQ ID NOs: 20, 21 and 22, each comprising at least a portion of CD64 ectodomain. In some embodiments, the hnCD16 comprises an amino acid sequence of at least 90% identity to any of SEQ ID NOs: 20-22. In some embodiments, the hnCD16 comprises an amino acid sequence of at least 95% identity to any of SEQ ID NOs: 20-22. In some embodiments, the hnCD16 comprises the amino acid sequence of SEQ ID NO: 20. In some embodiments, the hnCD16 comprises the amino acid sequence of SEQ ID NO: 21. In some embodiments, the hnCD16 comprises the amino acid sequence of SEQ ID NO: 22.

SEQ ID NO: 20
MWFLTTLLLWVPVDGQVDTTKAVITLQPPWVSVFQEETVTLHCEVLHLPG
SSSTQWFLNGTATQTSTPSYRITSASVNDSGEYRCQRGLSGRSDPIQLEI
HRGWLLLQVSSRVFTEGEPLALRCHAWKDKLVYNVLYYRNGKAFKFFHWN
SNLTILKTNISHNGTYHCSGMGKHRYTSAGISVTVKELFPAPVLNASVTS
PLLEGNLVTLSCETKLLLQRPGLQLYFSFYMGSKTLRGRNTSSEYQILTA
RREDSGLYWCEAATEDGNVLKRSPELELQVLGLQLPTPVWFHYQVSFCLV
MVLLFAVDTGLYFSVKTNIRSSTRDWKDHKFKWRKDPQDK
(340 a.a. CD64 domain-based construction; CD16TM;
CD16ICD)
SEQ ID NO: 21
MWFLTTLLLWVPVDGQVDTTKAVITLQPPWVSVFQEETVTLHCEVLHLPG
SSSTQWFLNGTATQTSTPSYRITSASVNDSGEYRCQRGLSGRSDPIQLEI
HRGWLLLQVSSRVFTEGEPLALRCHAWKDKLVYNVLYYRNGKAFKFFHWN
SNLTILKTNISHNGTYHCSGMGKHRYTSAGISVTVKELFPAPVLNASVTS
PLLEGNLVTLSCETKLLLQRPGLQLYFSFYMGSKTLRGRNTSSEYQILTA
RREDSGLYWCEAATEDGNVLKRSPELELQVLGLFFPPGYQVSFCLVMVLL
FAVDTGLYFSVKTNIRSSTRDWKDHKFKWRKDPQDK
(336 a.a. CD64 exon-based construction; CD16TM;
CD16ICD)
SEQ ID NO: 22
MWFLTTLLLWVPVDGQVDTTKAVITLQPPWVSVFQEETVTLHCEVLHLPG
SSSTQWFLNGTATQTSTPSYRITSASVNDSGEYRCQRGLSGRSDPIQLEI
HRGWLLLQVSSRVFTEGEPLALRCHAWKDKLVYNVLYYRNGKAFKFFHWN
SNLTILKTNISHNGTYHCSGMGKHRYTSAGISVTVKELFPAPVLNASVTS
PLLEGNLVTLSCETKLLLQRPGLQLYFSFYMGSKTLRGRNTSSEYQILTA
RREDSGLYWCEAATEDGNVLKRSPELELQVLGFFPPGYQVSFCLVMVLLF
AVDTGLYFSVKTNIRSSTRDWKDHKFKWRKDPQDK
(335 a.a. CD64 exon-based construction; CD16TM;
CD16ICD)

Accordingly, provided herein are iPSCs genetically engineered to comprise, among other editing as contemplated and described herein, an exogenous CD16 (“CD16^{exo}” in Table 1) that is a high-affinity non-cleavable CD16 receptor (hnCD16), wherein the genetically engineered iPSCs are capable of differentiating into effector cells comprising the hnCD16 introduced to the iPSCs. In some embodiments, the derived effector cells comprising a Fas redirector, and optionally a CAR and/or an exogenous CD16 are NK cells. In some embodiments, the derived effector cells comprising a Fas redirector, and optionally a CAR and/or an exogenous CD16 are T cells.

The exogenous hnCD16 expressed in iPSC or derivative cells thereof has high affinity in binding to not only ADCC antibodies or fragments thereof, but also to bi-, tri-, or multi-specific engagers or binders that recognize the CD16 or CD64 extracellular binding domains of the hnCD16. The bi-, tri-, or multi-specific engagers or binders are further described below in this application. As such, the present application provides a derivative effector cell or a cell population thereof, preloaded with one or more pre-selected ADCC antibodies through high-affinity binding with the extracellular domain of the hnCD16 or a variant thereof expressed on the derivative effector cell, in an amount sufficient for therapeutic use in a treatment of a condition, a disease, or an infection as further detailed below, wherein said hnCD16 comprises an extracellular binding domain of CD64, or of CD16 having F176V and S197P. Thus, in some embodiments, the derived NK cells are preloaded with an antibody. In some embodiments, the derived NK cells are used in a combination therapy with an antibody. In some embodiments, the antibody in the combination therapy or preloaded with the derived effector cells specifically targets CD38. In some embodiments, the antibody in the combination therapy or preloaded with the derived effector cells specifically targets an antigen different from CD38. In some embodiments, the anti-CD38 antibody is daratumumab.

In some other embodiments, the exogenous CD16 expressed in iPSC or derivative cells thereof comprises a CD16-, or variants thereof, based CFcR. A chimeric Fc receptor (CFcR) is produced to comprise a non-native transmembrane domain, a non-native stimulatory domain and/or a non-native signaling domain by modifying or replacing the native CD16 transmembrane- and/or the intracellular-domain. The term “non-native” used herein means that the transmembrane, stimulatory or signaling domain are derived from a different receptor other than the receptor which provides the extracellular domain. In the illustration here, the CFcR based on CD16 or variants thereof does not have a transmembrane, stimulatory or signaling domain that is derived from CD16. In some embodiments, the exogenous CD16-based CFcR comprises a non-native transmembrane domain derived from CD3δ, CD3ε, CD3γ, CD3ζ, CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor polypeptide. In some embodiments, the exogenous CD16-based CFcR comprises a non-native stimulatory/inhibitory domain derived from CD27, CD28, 4-1BB, OX40, ICOS, PD-1, LAG-3, 2B4, BTLA, DAP10, DAP12, CTLA-4, or NKG2D polypeptide. In some embodiments, the exogenous CD16-based CFcR comprises a non-native signaling domain derived from CD3ζ, 2B4, DAP10, DAP12, DNAM1, CD137 (4-1BB), IL21, IL7, IL12, IL15, NKp30, NKp44, NKp46, NKG2C, or NKG2D polypeptide. In some embodiments of the CD16-based CFcR, the provided chimeric Fc receptor comprises a transmembrane domain and a signaling domain both derived from one of IL7, IL12, IL15, NKp30, NKp44, NKp46, NKG2C, or NKG2D polypeptide. One particular exemplary embodiment of the CD16-based chimeric Fc receptor comprises a transmembrane domain of NKG2D, a stimulatory domain of 2B4, and a signaling domain of CD3ζ; wherein the extracellular domain of the CFcR is derived from a full length or partial sequence of the extracellular domain of CD64 or CD16, and wherein the extracellular domain of CD16 comprises F176V and S197P. Another exemplary embodiment of the CD16-based chimeric Fc receptor comprises a transmembrane domain and a signaling domain of CD3ζ; wherein the extracellular domain of the CFcR is derived from a full length or partial sequence of the extracellular domain of CD64 or CD16, and wherein the extracellular domain of CD16 comprises F176V and S197P.

The various embodiments of CD16-based chimeric Fc receptor as described above are capable of binding, with high affinity, to the Fc region of an antibody or fragment thereof; or to a bi-, tri-, or multi-specific engagers or binders that recognize the CD16 or CD64 extracellular binding domains of the chimeric Fc receptor. Upon binding, the stimulatory and/or signaling domains of the chimeric receptor enable the activation and cytokine secretion of the effector cells, and the killing of the tumor cells targeted by the antibody, or the bi-, tri-, or multi-specific engager or binder having a tumor antigen binding component as well as the Fc region. Without being limited by theory, through the non-native transmembrane, stimulatory and/or signaling domains, or through an engager binding to the ectodomain, of the CD16-based chimeric Fc receptor, the CFcR could contribute to effector cells' killing ability while increasing the effector cells' proliferation and/or expansion potential. The antibody and the engager can bring tumor cells expressing the antigen and the effector cells expressing the CFcR into a close proximity, which also contributes to the enhanced killing of the tumor cells. Exemplary tumor antigens for bi-, tri-, multi-specific engagers or binders include, but are not limited to, B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EPCAM, FLT-3, FOLR1, FOLR3, GD2, gpA33, HER2, HM1.24, LGR5, MSLN, MCSP, MICA/B, PSMA, PAMA, P-cadherin, and ROR1. Some non-limiting exemplary bi-, tri-, multi-specific engagers or binders suitable for engaging effector cells expressing the CD16-based CFcR in attacking tumor cells include CD16 (or CD64)-CD30, CD16 (or CD64)-BCMA, CD16 (or CD64)-IL15-EPCAM, and CD16 (or CD64)-IL15-CD33.

Accordingly, in some embodiments, the present invention provides iPSCs and derivative cells therefrom comprising an exogenous Fas redirector and an exogenous CD16 or a variant thereof, wherein the cells, including derivative T and NK cells, are useful for overcoming Fas induced apoptosis or fratricide. In some embodiments, the iPSC and derivative cells thereof comprise a Fas redirector and an exogenous CD16 or a variant thereof, and optionally a CAR. In some embodiments, the iPSC and derivative cells thereof comprise one or more additional genomic edits as described herein, without adversely impacting the differentiation potential of the iPSC and function of the derived effector cells including derivative T and NK cells. Also provided is a master cell bank comprising single cell sorted and expanded clonal engineered iPSCs having at least an exogenously introduced Fas redirector and an exogenous CD16 or a variant thereof, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 4. CD38 Knock-Out

The cell surface molecule CD38 is highly upregulated in multiple hematologic malignancies derived from both lymphoid and myeloid lineages, including multiple myeloma and a CD20 negative B-cell malignancy, which makes it an attractive target for antibody therapeutics to deplete cancer cells. Antibody mediated cancer cell depletion is usually attributable to a combination of direct cell apoptosis induction and activation of immune effector mechanisms such as ADCC (antibody-dependent cell-mediated cytotoxicity). In addition to ADCC, the immune effector mechanisms in concert with the therapeutic antibody may also include antibody-dependent cell-mediated phagocytosis (ADCP) and/or complement-dependent cytotoxicity (CDC).

Other than being highly expressed on malignant cells, CD38 is also expressed on plasma cells as well as on NK cells, and activated T and B cells. During hematopoiesis, CD38 is expressed on CD34⁺ stem cells and lineage-committed progenitors of lymphoid, erythroid, and myeloid, and during the final stages of maturation which continues through the plasma cell stage. As a type II transmembrane glycoprotein, CD38 carries out cell functions as both a receptor and a multifunctional enzyme involved in the production of nucleotide-metabolites. As an enzyme, CD38 catalyzes the synthesis and hydrolysis of the reaction from NAD⁺ to ADP-ribose, thereby producing secondary messengers CADPR and NAADP which stimulate release of calcium from the endoplasmic reticulum and lysosomes, critical for the calcium dependent process of cell adhesion. As a receptor, CD38 recognizes CD31 and regulates cytokine release and cytotoxicity in activated NK cells. CD38 is also reported to associate with cell surface proteins in lipid rafts, to regulate cytoplasmic Ca²⁺ flux, and to mediate signal transduction in lymphoid and myeloid cells.

In malignancy treatment, systemic use of CD38 antigen binding receptor transduced T cells however have been shown to lyse the CD38⁺ fractions of CD34⁺ hematopoietic progenitor cells, monocytes, NK cells, T cells and B cells, leading to incomplete treatment responses and reduced or eliminated efficacy because of the impaired recipient immune effector cell function. In addition, in multiple myeloma patients treated with daratumumab, a CD38-specific antibody, NK cell reduction in both bone marrow and peripheral blood was observed, although other immune cell types, such as T cells and B cells, were unaffected despite their CD38 expression (Casneuf et al., Blood Advances. 2017; 1(23):2105-2114).

Without being limited by theories, the present application includes a strategy to leverage the full potential of CD38-targeted cancer treatment by knocking out CD38 in the effector cell, thereby overcoming CD38-specific antibody and/or CD38 antigen binding domain-induced effector cell depletion or reduction through fratricide. In addition, since CD38 is upregulated on activated lymphocytes such as T or B cells, by suppressing activation of these recipient lymphocytes using a CD38-specific antibody, such as daratumumab, in the recipient of allogeneic effector cells, host allorejection against these effector cells would be reduced and/or prevented, thereby increasing effector cell survival and persistency. As such, a CD38-specific antibody, a secreted CD38-specific engager or a CD38-CAR (chimeric antigen receptor) against activation of recipient T, Treg, NK, and/or B cells can be used as a replacement for lymphodepletion using chemotherapy such as Cy/Flu (cyclophosphamide/fludarabine) prior to adoptive cell transferring. In addition, when targeting CD38⁺ T and pbNK cells using hnCD16a⁺/CD38⁻ effector cells in the presence of anti-CD38 antibodies or CD38 inhibitors, the depletion of CD38⁺ alloreactive cells increases the NAD⁺ (nicotinamide adenine dinucleotide, a substrate of CD38) availability and decreases NAD⁺ consumption related cell death, which, among other advantages, boosts effector cell responses in an immunosuppressive tumor microenvironment and supports cell rejuvenation in aging, degenerative or inflammatory diseases.

Thus, the strategies as provided herein also include generating an iPSC line comprising a Fas redirector and CD38 knock-out, and optionally one or more of a CAR and exogenous CD16 or a variant thereof, and obtaining derivative effector cells comprising a Fas redirector and CD38 null (CD38^{-/-}), and optionally one or more of a CAR and exogenous CD16 or a variant thereof, through directed differentiation of the engineered iPSC line. In one embodiment, the CD38 knock-out in an iPSC line is a bi-allelic knock-out.

As disclosed herein, in some embodiments, the provided iPSC line comprising a Fas redirector and CD38^{-/-}, and optionally one or more of a CAR and exogenous CD16, is capable of directed differentiation to produce functional derivative hematopoietic cells including, but not limited to, mesodermal cells with definitive hemogenic endothelium (HE) potential, definitive HE, CD34 hematopoietic cells, hematopoietic stem and progenitor cells, hematopoietic multipotent progenitors (MPP), T cell progenitors, NK cell progenitors, myeloid cells, neutrophil progenitors, T lineage cells, NKT lineage cells, NK lineage cells, B lineage cells, neutrophils, dendritic cells, and macrophages. In some embodiments, when an anti-CD38 antibody is used to induce CD16-mediated enhanced ADCC, or an anti-CD38 CAR is used for targeted cell killing, the iPSC comprising a Fas redirector and CD38^{-/-}, and optionally one or more of a CAR and exogenous CD16 and/or derivative effector cells thereof can target the CD38 expressing (tumor) cells without causing effector cell elimination, i.e., reduction or depletion of CD38 expressing effector cells, thereby increasing persistence and/or survival of the iPSC and its effector cell in the presence of, and/or after exposure to, such therapeutic agents. In some embodiments, the effector cell has increased persistence and/or survival in vivo in the presence of, and/or after exposure to, such anti-CD38 therapeutic agents, such as anti-CD38 antibodies. In some embodiments, the anti-CD38 antibody is daratumumab, isatuximab, or MOR202. In addition, since CD38 is upregulated on activated lymphocytes such as T or B cells, a CD38-specific antibody can be used for lymphodepletion thereby eliminating those activated lymphocytes, overcoming allorejection, increasing survival and persistency of the CD38 negative effector cells without fratricide in the recipient of the allogeneic effector cell therapy.

In some embodiments, the derived effector cells comprising a Fas redirector and CD38^{-/-}, and optionally one or more of a CAR and exogenous CD16 are NK cells derived from iPSCs. In some embodiments, the effector cells comprising a Fas redirector and CD38^{-/-}, and optionally one or more of a CAR and exogenous CD16 are T cells derived from iPSCs. In some embodiments, the iPSC comprising a Fas redirector and CD38^{-/-}, and optionally one or more of a CAR and exogenous CD16 and/or derivative cells thereof comprise one or more additional genomic edits as provided in Table 1, without adversely impacting the differentiation potential of the iPSC and function of the derived effector cells, including derivative T and NK cells. Also provided is a master cell bank comprising single cell sorted and expanded clonal engineered iPSCs having at least an exogenously introduced Fas redirector and CD38^{-/-}, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

5. Exogenously Introduced Cytokine and/or Cytokine Signaling

By avoiding systemic high-dose administration of clinically relevant cytokines, the risk of dose-limiting toxicities due to such a practice is reduced while cytokine-mediated cell autonomy is being established. To achieve lymphocyte autonomy without the need to additionally administer soluble cytokines, a cytokine signaling complex comprising a partial or full length peptide of one or more of IL2, IL4, IL6, IL7, IL9, IL10, IL11, IL12, IL15, IL18, IL21, and/or their respective receptors may be introduced to the cell to enable cytokine signaling with or without the expression of the cytokine itself, thereby maintaining or improving cell growth, proliferation, expansion, and/or effector function with reduced risk of cytokine toxicities. In some embodiments, the introduced cytokine and/or its respective native or modified receptor for cytokine signaling (signaling complex) are expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, the transient/temporal expression of a cell surface cytokine/cytokine receptor is through an expression construct carried by a retrovirus, Sendai virus, an adenovirus, an episome, mini-circle, or RNAs including mRNA.

Various construct designs for introducing a protein complex for signaling of one, two, or more cytokines including, but not limited to, IL2, IL4, IL6, IL7, IL9, IL10, IL11, IL12, IL15, IL18 and IL21, into the cell are provided herein. In embodiments where the cytokine signaling complex is for IL15, the transmembrane (TM) domain can be native to the IL15 receptor or may be modified or replaced with the transmembrane domain of any other membrane bound proteins. In some embodiments, IL15 and IL15Rα are co-expressed by using a self-cleaving peptide, mimicking trans-presentation of IL15, without eliminating cis-presentation of IL15. In other embodiments, IL15Rα is fused to IL15 at the C-terminus through a linker, mimicking trans-presentation without eliminating cis-presentation of IL15 as well as ensuring that IL15 is membrane-bound. In other embodiments, IL15Rα with a truncated intracellular domain is fused to IL15 at the C-terminus through a linker, mimicking trans-presentation of IL15, maintaining membrane-bound IL15, and additionally eliminating cis-presentation and/or any other potential signal transduction pathways mediated by a normal IL15R through its intracellular domain.

In various embodiments, such a truncated construct comprises an amino acid sequence of at least 75%, 80%, 85%, 90%, 95% or 99% identity to SEQ ID NO: 23. In some embodiments, the IL15/IL15Rα comprises an amino acid sequence of at least 90% sequence identity to SEQ ID NO: 23. In some embodiments, the IL15/IL15Rα comprises an amino acid sequence of at least 95% sequence identity to SEQ ID NO: 23. In some embodiments, the IL15/IL15Rα comprises the amino acid sequence of SEQ ID NO: 23. In one embodiment of the truncated IL15/IL15Rα, the construct does not comprise the last 4 amino acid residues (KSRQ) of SEQ ID NO: 23, and comprises an amino acid sequence of at least 75%, 80%, 85%, 90%, 95% or 99% identity to SEQ ID NO: 24. In some embodiments, the IL15/IL15Rα comprises an amino acid sequence of at least 90% sequence identity to SEQ ID NO: 24. In some embodiments, the IL15/IL15Rα comprises an amino acid sequence of at least 95% sequence identity to SEQ ID NO: 24. In some embodiments, the IL15/IL15Rα comprises the amino acid sequence of SEQ ID NO: 24.

SEQ ID NO: 23
MDWTWILFLVAAATRVHSGIHVFILGCFSAGLPKTEANWVNVISDLKKIE
DLIQSMHIDATLYTESDVHPSCKVTAMKCFLLELQVISLESGDASIHDTV
ENLIILANNSLSSNGNVTESGCKECEELEEKNIKEFLQSFVHIVQMFINT
SSGGGSGGGGSGGGGSGGGGSGGGSLQITCPPPMSVEHADIWVKSYSLYS
RERYICNSGFKRKAGTSSLTECVLNKATNVAHWTTPSLKCIRDPALVHQR
PAPPSTVTTAGVTPQPESLSPSGKEPAASSPSSNNTAATTAAIVPGSQLM
PSKSPSTGTTEISSHESSHGTPSQTTAKNWELTASASHQPPGVYPQGHSD
TTVAISTSTVLLCGLSAVSLLACYLKSRQ
(379 a.a.; variable signal and linker peptides are
underlined)
SEQ ID NO: 24
MDWTWILFLVAAATRVHSGIHVFILGCFSAGLPKTEANWVNVISDLKKIE
DLIQSMHIDATLYTESDVHPSCKVTAMKCFLLELQVISLESGDASIHDTV
ENLIILANNSLSSNGNVTESGCKECEELEEKNIKEFLQSFVHIVQMFINT
SSGGGSGGGGSGGGGSGGGGSGGGSLQITCPPPMSVEHADIWVKSYSLYS
RERYICNSGFKRKAGTSSLTECVLNKATNVAHWTTPSLKCIRDPALVHQR
PAPPSTVTTAGVTPQPESLSPSGKEPAASSPSSNNTAATTAAIVPGSQLM
PSKSPSTGTTEISSHESSHGTPSQTTAKNWELTASASHQPPGVYPQGHSD
TTVAISTSTVLLCGLSAVSLLACYL
(375 a.a.; variable signal and linker peptides are
underlined)

In yet other embodiments, the cytoplasmic domain of IL15Rα can be omitted without negatively impacting the autonomous feature of the effector cell equipped with IL15. In other embodiments, essentially the entire IL15Rα is removed except for the Sushi domain fused with IL15 at one end and a transmembrane domain on the other (mb-Sushi), optionally with a linker between the Sushi domain and the trans-membrane domain. The fused IL15/mb-Sushi is expressed at the cell surface through the transmembrane domain of any membrane bound protein. Thus, unnecessary signaling through IL15Rα, including cis-presentation, is eliminated when only the desirable trans-presentation of TL15 is retained. In some embodiments, the component comprising IL15 fused with Sushi domain comprises an amino acid sequence of at least 75%, 80%, 85%, 90%, 95% or 99% identity to SEQ ID NO: 25. In some embodiments, the component comprising IL15 fused with Sushi domain comprises an amino acid sequence of at least 90% identity to SEQ ID NO: 25. In some embodiments, the component comprising IL15 fused with Sushi domain comprises an amino acid sequence of at least 95% identity to SEQ ID NO: 25. In some embodiments, the component comprising IL15 fused with Sushi domain comprises the amino acid sequence of SEQ ID NO: 25.

SEQ ID NO: 25
MDWTWILFLVAAATRVHSGIHVFILGCFSAGLPKTEANWVNVISDLKKIE
DLIQSMHIDATLYTESDVHPSCKVTAMKCFLLELQVISLESGDASIHDTV
ENLIILANNSLSSNGNVTESGCKECEELEEKNIKEFLQSFVHIVQMFINT
SSGGGSGGGGSGGGGSGGGGSGGGSLQITCPPPMSVEHADIWVKSYSLYS
RERYICNSGFKRKAGTSSLTECVLNKATNVAHWTTPSLKCIR
(242 a.a .; signal and linker peptides are
underlined)

In other embodiments, a native or modified IL15Rβ is fused to IL15 at the C-terminus through a linker, enabling constitutive signaling and maintaining IL15 membrane-bound and trans-presentation. In other embodiments, a native or modified common receptor γC is fused to IL15 at the C-terminus through a linker for constitutive signaling and membrane bound trans-presentation of the cytokine. The common receptor γC is also called the common gamma chain or CD132, which is also known as IL2 receptor subunit gamma or IL2RG. γC is a cytokine receptor subunit that is common to the receptor complexes for many interleukin receptors, including, but not limited to, IL2, IL4, IL7, IL9, IL15 and IL21 receptors. In other embodiments, engineered IL15Rβ that forms a homodimer in the absence of IL15 is useful for producing constitutive signaling of the cytokine.

In other various embodiments, the cytokine signaling complex comprises an IL7 receptor fusion (IL7RF) comprising a full or partial length of IL7 and a full or partial length of IL7 receptor (IL7R). The transmembrane (TM) domain can be native to the IL7 receptor or may be modified or replaced with a transmembrane domain of any other membrane bound proteins. In one embodiment, a native (or wildtype) or modified IL7R is fused to IL7 at the C-terminus through a linker, enabling constitutive signaling and maintaining membrane-bound IL7. In some embodiments, such a construct comprises an amino acid sequence of at least 75%, 80%, 85%, 90%, 95% or 99% identity to SEQ ID NO: 26, with transmembrane domain, signal peptide and linker being flexible and varying in length and/or sequences. In some embodiments, the sequence identity is at least 80%. In some embodiments, the sequence identity is at least 90%. In some embodiments, the sequence identity is at least 95%. In some embodiments, the sequence identity is 100%.

SEQ ID NO: 26
MDWTWILFLVAAATRVHSDCDIEGKDGKQYESVLMVSIDQLLDSMKEIGS
NCLNNEFNFFKRHICDANKEGMFLFRAARKLRQFLKMNSTGDFDLHLLKV
SEGTTILLNCTGQVKGRKPAALGEAQPTKSLEENKSLKEQKKLNDLCFLK
RLLQEIKTCWNKILMGTKEHSGGGSGGGGSGGGGSGGGGSGGGS
LQESGYAQNGDLEDAELDDYSFSCYSQLEVNGSQHSLTCAFEDPDVNITN
LEFEICGALVEVKCLNFRKLQEIYFIETKKELLIGKSNICVKVGEKSLTC
KKIDLTTIVKPEAPFDLSVVYREGANDFVVTFNTSHLQKKYVKVLMHDVA
YRQEKDENKWTHVNLSSTKLTLLQRKLQPAAMYEIKVRSIPDHYFKGFWS
EWSPSYYFRTPEINNSSGEMDPILLTISILSFFSVALLVILACVLWKKRI
KPIVWPSLPDHKKTLEHLCKKPRKNLNVSFNPESFLDCQIHRVDDIQARD
EVEGFLQDTFPQQLEESEKQRLGGDVQSPNCPSEDVVITPESFGRDSSLT
CLAGNVSACDAPILSSSRSLDCRESGKNGPHVYQDLLLSLGTINSTLPPP
FSLQSGILTLNPVAQGQPILTSLGSNQEEAYVTMSSFYQNQ
(Signal peptide-IL7-linker-IL 7R; transmembrane
domain (TM), signal peptide and linker can vary in
length and sequences)

In one embodiment, a native or modified common receptor γC is fused to IL7 at the C-terminus through a linker for the constitutive and membrane-bound cytokine signaling complex. The common receptor γC is also called the common gamma chain or CD132, which is also known as IL2 receptor subunit gamma or IL2RG. γC is a cytokine receptor sub-unit that is common to the receptor complexes for many interleukin receptors, including, but not limited to, IL2, IL4, IL7, IL9, and IL21 receptor. In addition, engineered IL7R that forms a homodimer in the absence of IL7 is useful for producing constitutive signaling of the cytokine as well.

One having ordinary skill in the art would appreciate that the signal peptide and the linker sequences above are illustrative and in no way limit their variations suitable for use as a signal peptide or linker. There are many suitable signal peptide or linker sequences known and available to those in the art. The ordinary skilled in the art understands that the signal peptide and/or linker sequences may be substituted for another sequence without altering the activity of the functional peptide led by the signal peptide or linked by the linker. As such, the iPSC and derivative cells therefrom are capable of maintaining or improving cell growth, proliferation, expansion, and/or effector function autonomously without contacting additionally supplied soluble cytokines in vitro or in vivo.

In iPSCs and derivative cells therefrom comprising both a CAR and a cytokine signaling complex (“IL” in Table 1), the CAR and IL may be expressed in separate constructs, or may be co-expressed in a bi-cistronic construct comprising both CAR and IL. In some embodiments, the cytokine signaling complex can be linked to either the 5′ or the 3′ end of a CAR expression construct through a self-cleaving 2A coding sequence. As such, a cytokine signaling complex (e.g., IL7 signaling complex) and CAR may be in a single open reading frame (ORF). In one embodiment, the cytokine signaling complex is comprised in a CAR-2A-IL or IL-2A-CAR construct. When CAR-2A-IL or IL-2A-CAR is expressed, the self-cleaving 2A peptide allows the expressed CAR and IL to dissociate, and the dissociated IL can then be presented at the cell surface, with the transmembrane domain thereof anchored in the cell membrane. The CAR-2A-IL or IL-2A-CAR bi-cistronic construct design allows for coordinated CAR and IL expression both in timing and quantity, and under the same control mechanism that may be chosen to incorporate, for example, an inducible promoter or promoter with temporal or spatial specificity for the expression of the single ORF. Self-cleaving peptides are found in members of the Picornaviridae virus family, including aphthoviruses such as foot-and-mouth disease virus (FMDV), equine rhinitis A virus (ERAV), Thosea asigna virus (TaV) and porcine tescho virus-1 (PTV-I) (Donnelly, M L, et al, J. Gen. Virol, 82, 1027-101 (2001); Ryan, M D, et al., J. Gen. Virol., 72, 2727-2732 (2001)), and cardioviruses such as Theilovirus (e.g., Theiler's murine encephalomyelitis) and encephalomyocarditis viruses. The 2A peptides derived from FMDV, ERAV, PTV-I, and TaV are sometimes also referred to as “F2A”, “E2A”, “P2A”, and “T2A”, respectively.

The bi-cistronic CAR-2A-IL or IL-2A-CAR embodiment as disclosed herein is also contemplated for expression of any other cytokine or cytokine signaling complex provided herein, for example, IL2, IL4, IL6, IL9, IL10, IL11, IL12, IL18, and IL21. In some embodiments, the bi-cistronic CAR-2A-IL or IL-2A-CAR is for expression of one or more of IL2, IL4, IL7, IL9, IL15, IL21, and a cytokine signaling complex thereof.

As such, in various embodiments, a cytokine signaling complex as provided herein may be introduced to iPSC using one or more of the construct designs described above, and to its derivative cells upon iPSC differentiation. In addition to an iPSC, a clonal iPSC, a clonal iPS cell line, or iPSC-derived effector cells comprising at least one engineered modality as disclosed herein are provided. Accordingly, in some embodiments, the present invention provides iPSCs and derivative cells therefrom comprising a Fas redirector and an exogenous cytokine signaling complex described herein (“IL” in Table 1), and optionally one or more of a CAR, exogenous CD16 or a variant thereof, and CD38^{-/-}, wherein the Fas redirector redirects Fas signaling upon binding to a FAS agonist, thereby providing the cell, including derivative T and NK cells, with improved apoptosis resistance and/or exhaustion resistance. In some embodiments, the iPSC and derivative cells thereof comprise one or more additional genomic edits as provided in Table 1, without adversely impacting the differentiation potential of the iPSC and function of the derived effector cells, including derivative T and NK cells. Also provided is a master cell bank comprising single cell sorted and expanded clonal engineered iPSCs having at least a Fas redirector and an exogenously introduced cytokine signaling complex as described in this section, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

### 6. HLA-I- and HLA-II-Deficiency

Multiple HLA class I and class II proteins must be matched for histocompatibility in allogeneic recipients to avoid allogeneic rejection problems. Provided herein is an iPSC cell line with eliminated or substantially reduced expression of one or both HLA class I (“HLA-I”) and HLA class II (“HLA-II”) proteins. HLA class I deficiency can be achieved by functional deletion of any region of the HLA class I locus (chromosome 6p21), or deletion or reducing the expression level of HLA class-I associated genes including, not being limited to, beta-2 microglobulin (B2M) gene, TAP1 gene, TAP2 gene and Tapasin. For example, the B2M gene encodes a common subunit essential for cell surface expression of all HLA class I heterodimers. B2M negative cells are HLA-I deficient.

HLA class II deficiency can be achieved by functional deletion or reduction of HLA-II associated genes including, not being limited to, RFXANK, CIITA, RFX5 and RFXAP. CIITA is a transcriptional coactivator, functioning through activation of the transcription factor RFX5 required for class II protein expression. CIITA negative cells are HLA-II deficient. However, lacking HLA class I expression increases susceptibility to lysis by NK cells. As such, this application provides an iPSC and derivative cells therefrom comprising HLA-I and/or HLA-II deficiency, for example by lacking B2M and/or CIITA expression, wherein the obtained derivative effector cells enable allogeneic cell therapies by eliminating the need for MHC (major histocompatibility complex) matching, and avoiding recognition and killing by host (allogeneic) T cells.

Furthermore, a lack of HLA class I expression leads to lysis by host NK cells. Therefore, in addition to the above-discussed approach of CD38 conditioning to remove activated CD38-expressing host NK cells, to overcome this “missing self” response, HLA-G may be optionally knocked in to avoid NK cell recognition and killing of the HLA-I deficient effector cells derived from an engineered iPSC. In one embodiment, the provided HLA-I deficient iPSC and its derivative cells further comprise HLA-G knock-in. Alternatively, or in addition thereto, the provided HLA-I deficient iPSC and its derivative cells further comprise one or both of CD58 knockout and CD54 knockout. CD58 (or LFA-3) and CD54 (or ICAM-1) are adhesion proteins initiating signal-dependent cell interactions, and facilitating cell, including immune cell, migration. It was previously shown that CD58 and/or CD54 disruption effectively reduces the susceptibility of HLA-I deficient iPSC-derived effector cells to allogeneic NK cell killing. While it was shown that CD58 knockout has a higher efficiency in reducing allogeneic NK cell activation than CD54 knockout, double knockout of both CD58 and CD54 was shown to provide the most enhanced reduction of NK cell activation. In some observations, the CD58 and CD54 double knockout is even more effective than HLA-G overexpression for HLA-I deficient cells in overcoming “missing-self” effect.

Accordingly, in some embodiments, the present invention provides a strategy to enhance effector cell persistency and/or survival through reducing or preventing allorejection by generating HLA-I and/or HLA-II deficiency, optionally with additional HLA-I protein modifications, without adversely impacting the differentiation potential of the iPSC and function of the derived effector cells, including derivative T and NK cells.

In some embodiments, the effector cells have increased persistence and/or survival in vivo in the presence of, and/or after exposure to, therapeutic agents. Thus, in some embodiments, the iPSC and derivative cells thereof comprising a Fas redirector are HLA-I deficient (e.g., B2M negative or B2M^{-/-}), and optionally further comprise one or more of a CAR, exogenous CD16 or a variant thereof, CD38^{-/-}, and an exogenously introduced cytokine signaling complex (IL). In some embodiments, the iPSC and derivative cells thereof comprising a Fas redirector are HLA-I deficient and HLA-II deficient (e.g., B2M^{-/-} and CIITA negative or CIITA^{-/-}), and optionally further comprise one or more of a CAR, exogenous CD16 or a variant thereof, CD38^{-/-}, and IL. In some embodiments, the iPSC and derivative cells thereof comprising a Fas redirector are HLA-I and/or HLA-II deficient and further comprise an exogenous polynucleotide encoding HLA-G or HLA-E, and optionally one or more of a CAR, exogenous CD16 or a variant thereof, CD38^{-/-}, and IL. In some embodiments, the iPSC and derivative cells thereof comprising a Fas redirector are HLA-I and/or HLA-II deficient and are CD58 negative, and optionally further comprise one or more of a CAR, exogenous CD16 or a variant thereof, CD38^{-/-}, and IL. In some embodiments, the iPSC and derivative cells thereof comprising a Fas redirector are HLA-I and/or HLA-II deficient and are CD54 negative, and optionally further comprise one or more of a CAR, exogenous CD16 or a variant thereof, CD38^{-/-}, and IL.

In some embodiments, the iPSC and derivative cells thereof comprising a Fas redirector are HLA-I and/or HLA-II deficient and are both CD58 negative and CD54 negative, and optionally further comprise one or more of a CAR, exogenous CD16 or a variant thereof, CD38^{-/-}, and IL. In some embodiments, the effector cells comprising a Fas redirector and HLA-I and/or HLA-II deficiency, and optionally one or more of a CAR, exogenous CD16 or a variant thereof, CD38^{-/-}, and IL, are NK cells derived from iPSCs. In some embodiments, the effector cells comprising a Fas redirector and HLA-I and/or HLA-II deficiency, and optionally one or more of a CAR, exogenous CD16 or a variant thereof, CD38^{-/-}, and IL are T cells derived from iPSCs.

In some embodiments, the iPSC and derivative cells thereof comprising a Fas redirector and HLA deficiency or modification (e.g., HLA-I and/or HLA-II modification which comprises: HLA-I knockout with or without HLA-II knockout, and optionally one or more of HLA-E or HLA-G knockin or overexpression, CD54 knockout and CD58 knockout; such HLA-I and/or HLA-II modification is represented by “HLA” in Table 1), and optionally one or more of a CAR, exogenous CD16 or a variant thereof, CD38^{-/-}, and IL, said cells further comprise one or more additional genomic edits as provided in Table 1, without adversely impacting the differentiation potential of the iPSC and function of the derived effector cells, including derivative T and NK cells.

Also provided is a master cell bank comprising single cell sorted and expanded clonal engineered iPSCs having at least a Fas redirector and HLA deficiency as described in this section, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

7. Genetically Engineered iPSC Line and Derivative Cells Provided Herein

In light of the above, the present application provides an iPSC, a clonal iPS cell, an iPS cell line cell, or a derivative cell therefrom, wherein each cell comprises at least a Fas redirector (“Fas” in Table 1) as described herein. In some embodiments, the cells comprise an exogenous polynucleotide encoding a signaling redirector receptor that comprises (a) an extracellular Fas binding domain comprising a partial or full peptide of an extracellular domain (ECD) of a Fas receptor (FAS), or a variant or an allele thereof, and a cytoplasmic signaling domain comprising a partial or full peptide of an intracellular domain (ICD) of one or more co-stimulation molecules, wherein the signaling redirector receptor is a Fas redirector that redirects Fas signaling upon binding to a FAS agonist, thereby providing the cell or the derivative cell with improved apoptosis resistance and/or exhaustion resistance. In some embodiments, the derivative cells are hematopoietic lineage cells including, but are not limited to, mesodermal cells with definitive hemogenic endothelium (HE) potential, definitive HE, CD34 hematopoietic cells, hematopoietic stem and progenitor cells, hematopoietic multipotent progenitors (MPP), T cell progenitors, NK cell progenitors, myeloid cells, neutrophil progenitors, T lineage cells, NKT lineage cells, NK lineage cells, B lineage cells, neutrophils, dendritic cells, and macrophages. In some embodiments, the functional derivative hematopoietic cells comprise effector cells having one or more functional features that are not present in a counterpart primary T, NK, NKT, and/or B cell.

In some embodiments, the derivative cells comprise NK or T lineage cells. iPSC-derived NK or T lineage cells comprising a Fas redirector and optionally one or more of a CAR, exogenous CD16, CD38^{-/-}, a cytokine signaling complex, HLA deficiency, and any other modality, as shown in Table 1, are useful for overcoming or reducing Fas induced apoptosis or fratricide and/or tumor microenvironment suppression associated with solid tumors. As discussed above, derivative CAR-T cells expressing hnCD16 have acquired ADCC, providing an additional mechanism for tumor killing in addition to CAR targeting. In some embodiments, the derivative cells comprise NK lineage cells. iPSC-derived NK cells comprising a Fas redirector and optionally one or more of a CAR, exogenous CD16, CD38^{-/-}, a cytokine signaling complex, HLA deficiency, and any other modality, as shown in Table 1, have enhanced cytotoxicity, are effective in recruiting by-stander cells including T cells to infiltrate and kill tumor cells.

In some embodiments of effector cells comprising a Fas redirector and optionally one or more of a CAR, exogenous CD16, CD38^{-/-}, a cytokine signaling complex, HLA deficiency, and any other modality, as shown in Table 1, derived from engineered iPSC, the cells are intact in HLA-II and still withstand allorejection by activated recipient T, B, and NK cells. In some embodiments, the iPSC and its derivative effector cells are intact in HLA-II and have synergistically increased persistence and/or survival in the presence of activated recipient T, B, and NK cells. In some embodiments, the iPSC and its derivative effector cells are useful for overcoming or reducing tumor microenvironment suppression associated with solid tumors. When an anti-CD38 antibody is used with said derivative effector cells in a combination therapy, said cells have synergistically increased persistence, survival and effector function.

Also provided is an iPSC comprising a Fas redirector and optionally one or more of a CAR, exogenous CD16, CD38^{-/-}, a cytokine signaling complex (“IL” in Table 1), HLA deficiency, and any other modality as shown in Table 1, wherein the iPSC is capable of directed differentiation to produce functional derivative hematopoietic cells optionally with HLA-E or HLA-G knockin, and knockout of one or both of CD54 and CD58, to overcome alloreactive NK cells.

As such, the present application provides iPSCs and their functional derivative hematopoietic cells, which comprise any one of the following genotypes in Table 1. In some embodiments, iPSCs and their functional derivative hematopoietic cells have a genotype comprising at least a Fas redirector (“Fas” in Table 1). In addition, “HLA” in Table 1 represents HLA-I and/or HLA-II modification which comprises: HLA-I knockout with or without HLA-II knockout, and optionally one or more of HLA-E or HLA-G knockin or overexpression, CD54 knockout and CD58 knockout. In some embodiments, iPSCs and their functional derivative hematopoietic cells have a genotype comprising at least a Fas redirector and optionally a checkpoint inhibitor (“CI⁺ in Table 1), engager (“En⁺ in Table 1), or an antibody (“Ab⁺ in Table 1). Checkpoint inhibitors, engagers, and antibodies will be discussed in greater detail below.

TABLE 1
Applicable Exemplary Genotypes of the Cells Provided:

| Fas | CAR | CD16^{exo} | CD38^{-/-} | IL | HLA | Cl⁺/En⁺/Ab⁺ | Genotype | |
|---|---|---|---|---|---|---|---|---|
| √ | | | | | | | 1. | Fas |
| √ | √ | | | | | | 2. | Fas CAR |
| √ | | √ | | | | | 3. | Fas CD16^{exo} |
| √ | | | √ | | | | 4. | Fas CD38^{-/-} |
| √ | | | | √ | | | 5. | Fas IL |
| √ | | | | | √ | | 6. | Fas HLA |
| √ | | | | | | √ | 7. | Fas Cl⁺ |
| | | | | | | | 8. | Fas En⁺ |
| | | | | | | | 9. | Fas Ab⁺ |
| √ | √ | √ | | | | | 10. | Fas CAR CD16^{exo} |
| √ | √ | | √ | | | | 11. | Fas CAR CD38^{-/-} |
| √ | √ | | | √ | | | 12. | Fas CAR IL |
| √ | √ | | | | √ | | 13. | Fas CAR HLA |
| √ | √ | | | | | √ | 14. | Fas CAR Cl⁺ |
| | | | | | | | 15. | Fas CAR En⁺ |
| | | | | | | | 16. | Fas CAR Ab⁺ |
| √ | | √ | √ | | | | 17. | Fas CD16^{exo} CD38^{-/-} |
| √ | | √ | | √ | | | 18. | Fas CD16^{exo} IL |
| √ | | √ | | | √ | | 19. | Fas CD16^{exo} HLA |
| √ | | √ | | | | √ | 20. | Fas CD16^{exo} Cl⁺ |
| | | | | | | | 21. | Fas CD16^{exo} En⁺ |
| | | | | | | | 22. | Fas CD16^{exo} Ab⁺ |
| √ | | | √ | √ | | | 23. | Fas CD38^{-/-} IL |
| √ | | | √ | | √ | | 24. | Fas CD38^{-/-} HLA |
| √ | | | √ | | | √ | 25. | Fas CD38^{-/-} Cl⁺ |
| | | | | | | | 26. | Fas CD38^{-/-} En⁺ |
| | | | | | | | 27. | Fas CD38^{-/-} Ab⁺ |
| √ | | | | √ | √ | | 28. | Fas IL HLA |
| √ | | | | √ | | √ | 29. | Fas IL Cl⁺ |
| | | | | | | | 30. | Fas IL En⁺ |
| | | | | | | | 31. | Fas IL Ab⁺ |
| √ | | | | | √ | √ | 32. | Fas HLA Cl⁺ |
| | | | | | | | 33. | Fas HLA En⁺ |
| | | | | | | | 34. | Fas HLA Ab⁺ |
| √ | √ | √ | √ | | | | 35. | Fas CAR CD16^{exo} CD38^{-/-} |
| √ | √ | √ | | √ | | | 36. | Fas CAR CD16^{exo} IL |
| √ | √ | √ | | | √ | | 37. | Fas CAR CD16^{exo} HLA |
| √ | √ | √ | | | | √ | 38. | Fas CAR CD16^{exo} Cl⁺ |
| | | | | | | | 39. | Fas CAR CD16^{exo} En⁺ |
| | | | | | | | 40. | Fas CAR CD16^{exo} Ab⁺ |
| √ | √ | | √ | √ | | | 41. | Fas CAR CD38^{-/-} IL |
| √ | √ | | √ | | √ | | 42. | Fas CAR CD38^{-/-} HLA |
| √ | √ | | √ | | | √ | 43. | Fas CAR CD38^{-/-} Cl⁺ |
| | | | | | | | 44. | Fas CAR CD38^{-/-} En⁺ |
| | | | | | | | 45. | Fas CAR CD38^{-/-} Ab⁺ |
| √ | √ | | | √ | √ | | 46. | Fas CAR IL HLA |
| v | √ | | | √ | | √ | 47. | Fas CAR IL Cl⁺ |
| | | | | | | | 48. | Fas CAR IL En⁺ |
| | | | | | | | 49. | Fas CAR IL Ab⁺ |
| √ | √ | | | | √ | √ | 50. | Fas CAR HLA Cl⁺ |
| | | | | | | | 51. | Fas CAR HLA En⁺ |
| | | | | | | | 52. | Fas CAR HLA Ab⁺ |
| √ | | √ | √ | √ | | | 53. | Fas CD16^{exo} CD38^{-/-} IL |
| √ | | √ | √ | | √ | | 54. | Fas CD16^{exo} CD38^{-/-} HLA |
| √ | | √ | √ | | | √ | 55. | Fas CD16^{exo} CD38^{-/-} Cl⁺ |
| | | | | | | | 56. | Fas CD16^{exo} CD38^{-/-} En⁺ |
| | | | | | | | 57. | Fas CD16^{exo} CD38^{-/-} Ab⁺ |
| √ | | √ | | √ | √ | | 58. | Fas CD16^{exo} IL HLA |
| √ | | √ | | √ | | √ | 59. | Fas CD16^{exo} IL Ab⁺ |
| | | | | | | | 60. | Fas CD16^{exo} IL Cl⁺ |
| | | | | | | | 61. | Fas CD16^{exo} IL En⁺ |
| √ | | √ | | | √ | √ | 62. | Fas CD16^{exo} HLA Ab⁺ |
| | | | | | | | 63. | Fas CD16^{exo} HLA Cl⁺ |
| | | | | | | | 64. | Fas CD16^{exo} HLA En⁺ |
| √ | | | √ | √ | √ | | 65. | Fas CD38^{-/-} IL HLA |
| √ | | | √ | √ | | √ | 66. | Fas CD38^{-/-} IL Cl⁺ |
| | | | | | | | 67. | Fas CD38^{-/-} IL En⁺ |
| | | | | | | | 68. | Fas CD38^{-/-} IL Ab⁺ |
| √ | | | | √ | √ | √ | 69. | Fas IL HLA Cl⁺ |
| | | | | | | | 70. | Fas IL HLA En⁺ |
| | | | | | | | 71. | Fas IL HLA Ab⁺ |
| √ | √ | √ | √ | √ | | | 72. | Fas CAR CD16^{exo} CD38^{-/-} IL |
| √ | √ | √ | √ | | √ | | 73. | Fas CAR CD16^{exo} CD38^{-/-} HLA |
| √ | √ | √ | √ | | | √ | 74. | Fas CAR CD16^{exo} CD38^{-/-} Cl⁺ |
| | | | | | | | 75. | Fas CAR CD16^{exo} CD38^{-/-} En⁺ |
| | | | | | | | 76. | Fas CAR CD16^{exo} CD38^{-/-} Ab⁺ |
| √ | √ | √ | | √ | √ | | 77. | Fas CAR CD16^{exo} IL HLA |
| √ | √ | √ | | √ | | √ | 78. | Fas CAR CD16^{exo} IL Cl⁺ |
| | | | | | | | 79. | Fas CAR CD16^{exo} IL En⁺ |
| | | | | | | | 80. | Fas CAR CD16^{exo} IL Ab⁺ |
| √ | √ | √ | | | √ | √ | 81. | Fas CAR CD16^{exo} HLA Cl⁺ |
| | | | | | | | 82. | Fas CAR CD16^{exo} HLA En⁺ |
| | | | | | | | 83. | Fas CAR CD16^{exo} HLA Ab⁺ |
| √ | √ | | √ | √ | √ | | 84. | Fas CAR CD38^{-/-}IL HLA |
| √ | √ | | √ | √ | | √ | 85. | Fas CAR CD38^{-/-}IL Cl⁺ |
| | | | | | | | 86. | Fas CAR CD38^{-/-}IL En⁺ |
| | | | | | | | 87. | Fas CAR CD38^{-/-}IL Ab⁺ |
| √ | √ | | | √ | √ | √ | 88. | Fas CAR IL HLA Cl⁺ |
| | | | | | | | 89. | Fas CAR IL HLA En⁺ |
| | | | | | | | 90. | Fas CAR IL HLA Ab⁺ |
| √ | | √ | √ | √ | √ | | 91. | Fas CD16^{exo} CD38^{-/-} IL HLA |
| √ | | √ | √ | √ | | √ | 92. | Fas CD16^{exo} CD38^{-/-} IL Cl⁺ |
| | | | | | | | 93. | Fas CD16^{exo} CD38^{-/-} IL En⁺ |
| | | | | | | | 94. | Fas CD16^{exo} CD38^{-/-} IL Ab⁺ |
| √ | | √ | | √ | √ | √ | 95. | Fas CD16^{exo} IL HLA Cl⁺ |
| | | | | | | | 96. | Fas CD16^{exo} IL HLA En⁺ |
| | | | | | | | 97. | Fas CD16^{exo} IL HLA Ab⁺ |
| √ | | √ | √ | | √ | √ | 98. | Fas CD16^{exo} CD38^{-/-} HLA Cl⁺ |
| | | | | | | | 99. | Fas CD16^{exo} CD38^{-/-} HLA En⁺ |
| | | | | | | | 100. | Fas CD16^{exo} CD38^{-/-} HLA Ab⁺ |
| √ | | | √ | √ | √ | √ | 101. | IL CD38^{-/-} HLA Ab⁺ En⁺ |
| | | | | | | | 102. | IL CD38^{-/-} HLA Cl⁺ En⁺ |
| √ | √ | √ | √ | √ | √ | | 103. | Fas CAR CD16^{exo} CD38^{-/-} IL HLA |
| √ | √ | √ | √ | √ | | √ | 104. | Fas CAR CD16^{exo} CD38^{-/-} IL Cl⁺ |
| | | | | | | | 105. | Fas CAR CD16^{exo} CD38^{-/-} IL En⁺ |
| | | | | | | | 106. | Fas CAR CD16^{exo} CD38^{-/-} IL Ab⁺ |
| √ | √ | √ | | √ | √ | √ | 107. | Fas CAR CD16^{exo} IL HLA Cl⁺ |
| | | | | | | | 108. | Fas CAR CD16^{exo} IL HLA En⁺ |
| | | | | | | | 109. | Fas CAR CD16^{exo} IL HLA Ab⁺ |
| √ | √ | √ | √ | | √ | √ | 110. | Fas CAR CD16^{exo} CD38^{-/-} HLA Cl⁺ |
| | | | | | | | 111. | Fas CAR CD16^{exo} CD38^{-/-} HLA |
| | | | | | | | | En⁺ |
| | | | | | | | 112. | Fas CAR CD16^{exo} CD38^{-/-} HLA |
| | | | | | | | | Ab⁺ |
| √ | √ | | √ | √ | √ | √ | 113. | Fas CAR CD38^{-/-} IL HLA Cl⁺ |
| | | | | | | | 114. | Fas CAR CD38^{-/-} IL HLA En⁺ |
| | | | | | | | 115. | Fas CAR CD38^{-/-} IL HLA Ab⁺ |
| √ | | √ | √ | √ | √ | √ | 116. | Fas CD16^{exo} CD38^{-/-} IL HLA Cl⁺ |
| | | | | | | | 117. | Fas CD16^{exo} CD38^{-/-} IL HLA En⁺ |
| | | | | | | | 118. | Fas CD16^{exo} CD38^{-/-} IL HLA Ab⁺ |
| √ | √ | √ | √ | √ | √ | √ | 119. | Fas CAR CD16^{exo} CD38^{-/-} IL HLA |
| | | | | | | | | Cl⁺ |
| | | | | | | | 120. | Fas CAR CD16^{exo} CD38^{-/-} IL HLA |
| | | | | | | | | En⁺ |
| | | | | | | | 121. | Fas CAR CD16^{exo} CD38^{-/-} IL HLA |
| | | | | | | | | Ab⁺ |

### 8. Additional Modifications

In some embodiments, the genetically modified iPSC and the derivative cells thereof comprise a genotype listed in Table 1, wherein said cells may further comprise one or more of the following genetically modified modalities: safety switch proteins, targeting modalities, receptors, signaling molecules, transcription factors, pharmaceutically active proteins and peptides, drug target candidates; or proteins promoting engraftment, trafficking, homing, viability, self-renewal, persistence, immune response regulation and modulation, and/or survival of the iPSCs or derivative cells thereof. In some embodiments, the iPSC, and its derivative effector cells comprising any one of the genotypes in Table 1 may additionally comprise deletion or disruption of at least one of TAP1, TAP2, Tapasin, NLRC5, PD1, LAG3, TIM3, RFXANK, RFX5, RFXAP, RAG1, and any gene in the chromosome 6p21 region; or introduction of at least one of HLA-E, HLA-G, 4-1BBL, CD3, CD4, CD8, CD47, CD113, CD131, CD137, CD80, PDL1, A_{2A}R, TCR, Fc receptor, antibodies, engagers, and surface triggering receptors for coupling with bi-, multi-specific or universal engagers.

### 8.1 Engagers

Engagers are fusion proteins consisting of two or more single-chain variable fragments (scFvs), or other functional variants, of different antibodies or fragments thereof, with at least one scFv that binds to an effector cell surface molecule or surface triggering receptor, and at least another to a target cell via a target cell specific surface molecule. Engagers can be bi-specific or multi-specific. Such bi-specific or multi-specific engagers are capable of directing an effector cell (e.g., a T cell, a NK cell, an NKT cell, a B cell, a macrophage, and/or a neutrophil) to a tumor cell and activating the immune effector cell, and have shown great potential to maximize the benefits of CAR-T cell therapy. Examples of engagers include, but are not limited to, bi-specific T cell engagers (BiTEs), bi-specific killer cell engagers (BiKEs), tri-specific killer cell engagers (TriKEs), multi-specific killer cell engagers, or universal engagers compatible with multiple immune cell types.

In some embodiments, the engager is used in combination with a population of the effector cells described herein by concurrent or consecutive administration, wherein the effector cells comprise a surface molecule, or surface triggering receptor, that is recognized by the engager. In some other embodiments, the engager (“En⁺” in Table 1) is a bi-specific antibody expressed by a derivative effector cell through genetically engineering an iPSC as described herein, and directed differentiation of the engineered iPSC. Exemplary effector cell surface molecules, or surface triggering receptors, that can be used for bi- or multi-specific engager recognition, or coupling thereof, include, but are not limited to, CD3, CD5, CD16, CD28, CD32, CD33, CD64, CD89, NKG2C, NKG2D, and a chimeric Fc receptor as disclosed herein.

In some embodiments, the target cell for an engager is a tumor cell. The exemplary tumor cell surface molecules for bi- or multi-specific engager recognition include, but are not limited to, B7H3, BCMA, CD10, CD19, CD20, CD22, CD24, CD30, CD33, CD34, CD38, CD44, CD52, CD79a, CD79b, CD123, CD138, CD179b, CEA, CLEC12A, CS-1, DLL3, EGFR, EGFRvIII, EPCAM, FLT-3, FOLR1, FOLR3, GD2, gpA33, HER2, HM1.24, LGR5, MSLN, MCSP, MICA/B, Muc1, Muc16, PDL1, PSMA, PAMA, P-cadherin, ROR1, and VEGF-R2. In various embodiments, the bi-specific engager is a bi-specific antibody. Exemplary bi-specific antibodies include, but are not limited to, CD3-CD19 (specific to CD3 and CD19), CD16-CD30, CD64-CD30, CD16-BCMA, CD64-BCMA and CD3-CD33.

In yet another embodiment, the bi-specific antibody further comprises a linker between the effector cell and tumor cell antigen binding domains. For example, a modified IL15 may be used as a linker for effector NK cells to facilitate cell expansion (called TriKE, or Tri-specific Killer Engager, in some publications). Exemplary TriKEs include, but are not limited to, CD16-IL15-EPCAM, CD64-IL15-EPCAM, CD16-IL15-CD33, CD64-IL15-CD33, and NKG2C-IL15-CD33. In various embodiments, the IL15 in the TriKE may also originate from other cytokines including, but not limited to, IL2, IL4, IL6, IL7, IL9, IL10, IL11, IL12, IL18, and IL21.

In some embodiments, the surface triggering receptor for a bi- or multi-specific engager could be endogenous to the effector cells, sometimes depending on the cell types. In some other embodiments, one or more exogenous surface triggering receptors could be introduced to the effector cells using the methods and compositions provided herein, e.g., through additional engineering of an iPSC comprising a genotype listed in Table 1, then directing the differentiation of the iPSC to T, NK or any other effector cells comprising the same genotype and the surface triggering receptor as the source iPSC.

### 8.2 Antibodies for Immunotherapy

In some embodiments, in addition to the genomically engineered effector cells as provided herein, additional therapeutic agents comprising an antibody, or an antibody fragment that targets an antigen associated with a condition, a disease, or an indication may be used with these effector cells in a combinational therapy. In some embodiments, the antibody is used in combination with a population of the effector cells described herein by concurrent or consecutive administration to a subject. In other embodiments, such antibody or a fragment thereof (“Ab⁺” in Table 1) may be expressed by the effector cells by genetically engineering an iPSC using an exogenous polynucleotide sequence encoding the antibody or fragment thereof, and directing differentiation of the engineered iPSC. In some embodiments, the effector cell additionally expresses an exogenous CD16 variant, wherein the cytotoxicity of the effector cell is enhanced by the antibody via ADCC. In some embodiments, the antibody is a monoclonal antibody. In some embodiments, the antibody is a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor- or viral-specific antigen activates the administered iPSC-derived effector cells to enhance their killing ability. In some embodiments, the antibodies suitable for combinational treatment as an additional therapeutic agent to the administered iPSC-derived effector cells include, but are not limited to, anti-CD20 (rituximab, veltuzumab, ofatumumab, ublituximab, ocaratuzumab, obinutuzumab), anti-HER2 (trastuzumab, pertuzumab), anti-CD52 (alemtuzumab), anti-EGFR (cetuximab), anti-GD2 (dinutuximab), anti-PDL1 (avelumab), anti-CD38 (daratumumab, isatuximab, MOR202), anti-CD123 (7G3, CSL362), anti-SLAMF7 (elotuzumab); and their humanized or Fc modified variants or fragments, or their functional equivalents and biosimilars.

In some embodiments, the iPSC-derived effector cells, including iPSC-derived NK or T cells, comprising a Fas redirector and optionally one or more of a CAR, exogenous CD16, CD38^{-/-}, a cytokine signaling complex, and HLA deficiency, are used in a combination therapy comprising said effector cells and an anti-CD38 antibodies, such as daratumumab, isatuximab, and MOR202. In some embodiments of a combination useful for treating liquid or solid tumors, the combination comprises iPSC-derived NK or T cells comprising a Fas redirector and optionally one or more of a CAR, exogenous CD16, CD38^{-/-}, a cytokine signaling complex, and HLA deficiency; and daratumumab. In some further embodiments, the iPSC-derived NK cells comprised in the combination with one of the anti-CD38 antibodies, comprise a Fas redirector, CAR, exogenous CD16, CD38^{-/-}, HLA deficiency and IL15 or a cytokine signaling complex; wherein the IL15 or its signaling complex is co- or separately expressed with the CAR. In some further embodiments, the iPSC-derived T cells comprised in the combination with one of the anti-CD38 antibodies comprise a Fas redirector, CAR, exogenous CD16, CD38^{-/-} HLA deficiency and IL7 or a cytokine signaling complex thereof. In some particular embodiments, IL7 or its signaling complex is co- or separately expressed with the CAR.

In some embodiments, the iPSC-derived effector cells, including iPSC-derived NK or T cells, comprise a Fas redirector and optionally one or more of a CAR, exogenous CD16, CD38^{-/-}, a cytokine signaling complex, HLA deficiency, and Ab⁺, where “Ab⁺” refers to an exogenous polynucleotide sequence encoding the antibody or fragment thereof, and wherein the antibody or a fragment thereof is expressed by the effector cells. In various embodiments, the Ab⁺ comprises any one or more the therapeutic antibodies described above or functional fragments thereof.

### 8.3 Checkpoint Inhibitors

Checkpoints are cell molecules, often cell surface molecules, capable of suppressing or downregulating immune responses when not inhibited. It is now clear that tumors co-opt certain immune-checkpoint pathways as a major mechanism of immune resistance, particularly against T cells that are specific for tumor antigens. Checkpoint inhibitors (CIs) are antagonists capable of reducing checkpoint gene expression or gene products, or decreasing activity of checkpoint molecules, thereby blocking inhibitory checkpoints, and restoring immune system function. The development of checkpoint inhibitors targeting PD1/PDL1 or CTLA4 has transformed the oncology landscape, with these agents providing long term remissions in multiple indications. However, many tumor subtypes are resistant to checkpoint blockade therapy, and relapse remains a significant concern.

The present application therefore provides, in some embodiments, a therapeutic approach to overcome CI resistance by including genomically-engineered functional derivative cells as provided herein in a combination therapy with CI. In some embodiments, the checkpoint inhibitor is used in combination with a population of the effector cells described herein by concurrent or consecutive administration thereof to a subject. Some embodiments of the combination therapy with the effector cells described herein comprise at least one checkpoint inhibitor to target at least one checkpoint molecule; wherein the derivative cells have a genotype listed in Table 1. In other embodiments, the checkpoint inhibitor (“CIE” in Table 1) is expressed by the effector cells by genetically engineering an iPSC using an exogenous polynucleotide sequence encoding the checkpoint inhibitor, or a fragment or variant thereof, and directing differentiation of the engineered iPSC.

In some embodiments, the exogenous polynucleotide sequence encoding the checkpoint inhibitor, or a fragment thereof, is co-expressed with a CAR, either in separate constructs or in a bi-cistronic construct. In some further embodiments, the sequence encoding the checkpoint inhibitor, or the fragment thereof, can be linked to either the 5′ or the 3′ end of a CAR expression construct through a self-cleaving 2A coding sequence, illustrated as, for example, CAR-2A-CI or CI-2A-CAR. As such, the coding sequences of the checkpoint inhibitor and the CAR are in a single open reading frame (ORF). When the checkpoint inhibitor is delivered, expressed, and secreted as a payload by the derivative effector cells capable of infiltrating the tumor microenvironment (TME), it counteracts the inhibitory checkpoint molecule upon engaging the TME, allowing activation of the effector cells by activating modalities such as CAR or activating receptors. In one embodiment of the combination therapy, the derivative effector cells comprising a genotype listed in Table 1 are NK lineage cells. In another embodiment of the combination therapy, the derivative effector cells comprising a genotype listed in Table 1 are T lineage cells.

Suitable checkpoint inhibitors for combination therapy with the derivative effector cells and/or for expression by the derivative effector cells as provided herein include, but are not limited to, antagonists of PD-1 (Pdcdl, CD279), PDL-1 (CD274), TIM-3 (Havcr2), TIGIT (WUCAM and Vstm3), LAG-3 (Lag3, CD223), CTLA-4 (Ctla4, CD152), 2B4 (CD244), 4-1BB (CD137), 4-1BBL (CD137L), A_{2A}R, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR (for example, 2DL1, 2DL2, 2DL3, 3DL1, and 3DL2).

In some embodiments, the antagonist inhibiting any of the above checkpoint molecules is an antibody. In some embodiments, the checkpoint inhibitory antibodies may be murine antibodies, human antibodies, humanized antibodies, a camel Ig, a shark heavy-chain-only antibody (VNAR), Ig NAR, chimeric antibodies, recombinant antibodies, or antibody fragments thereof. Non-limiting examples of antibody fragments include Fab, Fab′, F(ab′)2, F(ab′)3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the checkpoint inhibitors comprise at least one of atezolizumab (anti-PDL1 mAb), avelumab (anti-PDL1 mAb), durvalumab (anti-PDL1 mAb), tremelimumab (anti-CTLA4 mAb), ipilimumab (anti-CTLA4 mAb), IPH4102 (anti-KIR), IPH43 (anti-MICA), IPH33 (anti-TLR3), lirimumab (anti-KIR), monalizumab (anti-NKG2A), nivolumab (anti-PD1 mAb), pembrolizumab (anti-PD1 mAb), and any derivatives, functional equivalents, or biosimilars thereof.

In some embodiments, the antagonist inhibiting any of the above checkpoint molecules is microRNA-based, as many miRNAs are found as regulators that control the expression of immune checkpoints (Dragomir et al., Cancer Biol Med. 2018, 15(2):103-115). In some embodiments, the checkpoint antagonistic miRNAs include, but are not limited to, miR-28, miR-15/16, miR-138, miR-342, miR-20b, miR-21, miR-130b, miR-34a, miR-197, miR-200c, miR-200, miR-17-5p, miR-570, miR-424, miR-155, miR-574-3p, miR-513, and miR-29c.

In some embodiments, the checkpoint inhibitor (“CI⁺” in Table 1) is co-expressed with CAR and inhibits at least one of the following checkpoint molecules: PD-1, PDL-1, TIM-3, TIGIT, LAG-3, CTLA-4, 2B4, 4-1BB, 4-1BBL, A_{2A}R, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/ILA-E, and inhibitory KIR. In some embodiments, the checkpoint inhibitor co-expressed with CAR in a derivative cell having a genotype listed in Table 1 is selected from the group comprising atezolizumab, avelumab, durvalumab, tremelimumab, ipilimumab, IPH4102, IPH43, IPH33, lirimumab, monalizumab, nivolumab, pembrolizumab, and their humanized, or Fc modified variants, fragments and their functional equivalents or biosimilars. In some embodiments, the checkpoint inhibitor co-expressed with CAR is atezolizumab, or its humanized, or Fc modified variants, fragments or their functional equivalents or biosimilars. In some other embodiments, the checkpoint inhibitor co-expressed with CAR is nivolumab, or its humanized, or Fc modified variants, fragments or their functional equivalents or biosimilars. In some other embodiments, the checkpoint inhibitor co-expressed with CAR is pembrolizumab, or its humanized, or Fc modified variants, fragments or their functional equivalents or biosimilars.

In some other embodiments of the combination therapy comprising the derivative effector cells provided herein and at least one antibody inhibiting a checkpoint molecule, the antibody is not produced by, or in, the derivative cells and is additionally administered before, with, or after the administering of the derivative cells as provided herein. In some embodiments, the administering of one, two, three or more checkpoint inhibitors in a combination therapy with the provided derivative NK lineage cells or T lineage cells are simultaneous or sequential. In one embodiment of the combinational treatment, the checkpoint inhibitor included in the treatment is one or more of atezolizumab, avelumab, durvalumab, tremelimumab, ipilimumab, IPH4102, IPH43, IPH33, lirimumab, monalizumab, nivolumab, pembrolizumab, and their humanized or Fc modified variants, fragments and their functional equivalents or biosimilars. In some embodiments of the combination treatment, the checkpoint inhibitor included in the treatment is atezolizumab, or its humanized or Fc modified variant, fragment and its functional equivalent or biosimilar. In some embodiments of the combination treatment, the checkpoint inhibitor included in the treatment is nivolumab, or its humanized or Fc modified variant, fragment or its functional equivalent or biosimilar. In some embodiments of the combination treatment, the checkpoint inhibitor included in the treatment is pembrolizumab, or its humanized or Fc modified variant, fragment or its functional equivalent or biosimilar.

In some embodiments, the iPSC-derived effector cells, including iPSC-derived NK or T cells, comprise a Fas redirector and optionally one or more of a CAR, exogenous CD16, CD38^{-/-}, a cytokine signaling complex, HLA deficiency, and CI⁺, where “CI⁺” refers to an exogenous polynucleotide sequence encoding the checkpoint inhibitor or fragment thereof, and wherein the checkpoint inhibitor or a fragment thereof is expressed by the effector cells. In various embodiments, the CI⁺ comprises any one or more the therapeutic antibodies described above or functional fragments thereof.

II. Methods for Targeted Genome Editing at Selected Locus in iPSCs

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. Therefore, targeted editing may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and for reliable gene response control.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.” In some situations, the targeted integration site is intended to be within a coding region of a selected gene, and thus the targeted integration could disrupt the gene expression, resulting in simultaneous knock-in and knock-out (KI/KO) in one single editing step.

Inserting one or more transgenes at a selected position in a gene locus of interest (GOI) to knock-out the gene at the same time can be achieved. Gene loci suitable for simultaneous knock-in and knockout (KI/KO) include, but are not limited to, B2M, TAP1, TAP2, tapasin, NLRC5, CIITA, RFXANK, RFX5, RFXAP, TCRα or TCRβ constant region, NKG2A, NKG2D, CD38, CD25, CD69, CD71, CD44, CD58, CD54, CD56, CIS, CBL-B, SOCS2, PD1, CTLA4, LAG3, TIM3, and TIGIT. With respective site-specific targeting homology arms for position-selective insertion, it allows the transgene(s) to express either under an endogenous promoter at the site or under an exogenous promoter comprised in the construct. When two or more transgenes are to be inserted at a selected location in CD38 locus, a linker sequence, for example, a 2A linker or IRES, is placed between any two transgenes. The 2A linker encodes a self-cleaving peptide derived from, e.g., FMDV, ERAV, PTV-I, or TaV (referred to as “F2A”, “E2A”, “P2A”, and “T2A”, respectively), allowing for separate proteins to be produced from a single translation. In some embodiments, insulators are included in the construct to reduce the risk of transgene and/or exogenous promoter silencing. In various embodiments, the exogenous promoter may be CAG, or other constitutive, inducible, temporal-, tissue-, or cell type-specific promoters including, but not limited to CMV, EF1α, PGK, and UBC.

Available endonucleases capable of introducing specific and targeted DSBs include, but are not limited to, zinc-finger nucleases (ZFN), transcription activator-like effector nucleases (TALEN), RNA-guided CRISPR (Clustered Regular Interspaced Short Palindromic Repeats) systems. Additionally, the DICE (dual integrase cassette exchange) system utilizing phiC31 and Bxb1 integrases is also a promising tool for targeted integration.

ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but are not limited to, C₂H₂ zinc fingers, C₃H zinc fingers, and C₄ zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496, the complete disclosures of which are incorporated herein by reference. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the FokI nuclease with a zinc finger DNA binding domain.

A TALEN is a targeted nuclease comprising a nuclease fused to a TAL effector DNA binding domain. By “transcription activator-like effector DNA binding domain”, “TAL effector DNA binding domain”, or “TALE DNA binding domain”, it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in U.S. Pub. No. 2011/0145940, which is herein incorporated by reference. The most recognized example of a TALEN in the art is a fusion polypeptide of the FokI nuclease to a TAL effector DNA binding domain.

Another example of a targeted nuclease that finds use in the subject methods is a targeted Spo11 nuclease, a polypeptide comprising a Spo11 polypeptide having nuclease activity fused to a DNA binding domain, e.g., a zinc finger DNA binding domain, a TAL effector DNA binding domain, etc. that has specificity for a DNA sequence of interest.

Additional examples of targeted nucleases suitable for embodiments of the present invention include, but not limited to Bxb1, phiC31, R4, PhiBT1, and Wβ/SPBc/TP901-1, whether used individually or in combination.

Other non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like.

Using Cas9 as an example, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction.

DICE mediated insertion uses a pair of recombinases, for example, phiC31 and Bxb1, to provide unidirectional integration of an exogenous DNA that is tightly restricted to each enzymes' own small attB and attP recognition sites. Because these target att sites that are not naturally present in mammalian genomes, they must be first introduced into the genome, at the desired integration site. See, for example, U.S. Pub. No. 2015/0140665, the disclosure of which is incorporated herein by reference.

One aspect of the present invention provides a construct comprising one or more exogenous polynucleotides for targeted genome integration. In one embodiment, the construct further comprises a pair of homologous arms specific to a desired integration site, and the method of targeted integration comprises introducing the construct to cells to enable site specific homologous recombination by the cell host enzymatic machinery. In another embodiment, the method of targeted integration in a cell comprises introducing a construct comprising one or more exogenous polynucleotides to the cell and introducing a ZFN expression cassette comprising a DNA-binding domain specific to a desired integration site to the cell to enable a ZFN-mediated insertion. In yet another embodiment, the method of targeted integration in a cell comprises introducing a construct comprising one or more exogenous polynucleotides to the cell and introducing a TALEN expression cassette comprising a DNA-binding domain specific to a desired integration site to the cell to enable a TALEN-mediated insertion. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration.

Promising sites for targeted integration include, but are not limited to, safe harbor loci, or genomic safe harbor (GSH), which are intragenic or extragenic regions of the human genome that, theoretically, are able to accommodate predictable expression of newly integrated DNA without adverse effects on the host cell or organism. A useful safe harbor must permit sufficient transgene expression to yield desired levels of the vector-encoded protein or non-coding RNA. A safe harbor also must not predispose cells to malignant transformation nor alter cellular functions. For an integration site to be a potential safe harbor locus, it ideally needs to meet criteria including, but not limited to: absence of disruption of regulatory elements or genes, as judged by sequence annotation; is an intergenic region in a gene dense area, or a location at the convergence between two genes transcribed in opposite directions; keep distance to minimize the possibility of long-range interactions between vector-encoded transcriptional activators and the promoters of adjacent genes, particularly cancer-related and microRNA genes; and has apparently ubiquitous transcriptional activity, as reflected by broad spatial and temporal expressed sequence tag (EST) expression patterns, indicating ubiquitous transcriptional activity. This latter feature is especially important in stem cells, where during differentiation, chromatin remodeling typically leads to silencing of some loci and potential activation of others. Within the region suitable for exogenous insertion, a precise locus chosen for insertion should be devoid of repetitive elements and conserved sequences and to which primers for amplification of homology arms could easily be designed.

Suitable sites for human genome editing, or specifically, targeted integration, include, but are not limited to, the adeno-associated virus site 1 (AAVS1), the chemokine (CC motif) receptor 5 (CCR5) gene locus and the human orthologue of the mouse ROSA26 locus. Additionally, the human orthologue of the mouse H11 locus may also be a suitable site for insertion using the composition and method of targeted integration disclosed herein. Further, collagen and HTRP gene loci may also be used as safe harbor for targeted integration. However, validation of each selected site has been shown to be necessary especially in stem cells for specific integration events, and optimization of insertion strategy including promoter election, exogenous gene sequence and arrangement, and construct design is often needed.

For targeted in/dels, the editing site is often comprised in an endogenous gene whose expression and/or function is intended to be disrupted. In some embodiments, the endogenous gene comprising a targeted in/del is associated with immune response regulation and modulation. In some other embodiments, the endogenous gene comprising a targeted in/del is associated with targeting modality, receptors, signaling molecules, transcription factors, drug target candidates, immune response regulation and modulation, or proteins suppressing engraftment, trafficking, homing, viability, self-renewal, persistence, and/or survival of stem cells and/or progenitor cells, and the derived cells therefrom.

As such, one aspect of the present invention provides a method of targeted integration in a selected locus including genome safe harbor or a preselected locus known or proven to be safe and well-regulated for continuous or temporal gene expression such as, in the case of a T cell, a constant region of a T Cell Receptor (TCR). In one embodiment, the genome safe harbor for the method of targeted integration comprises one or more desired integration site comprising AAVS1, CCR5, ROSA26, collagen, HTRP, H11, GAPDH, TCR or RUNX1, or other loci meeting the criteria of a genome safe harbor. In one embodiment, the method of targeted integration in a cell comprises introducing a construct comprising one or more exogenous polynucleotides to the cell, and introducing a construct comprising a pair of homologous arms specific to a desired integration site and one or more exogenous sequence, to enable site specific homologous recombination by the cell host enzymatic machinery, wherein the desired integration site comprises AAVS1, CCR5, ROSA26, collagen, HTRP, H11, GAPDH, TCR or RUNX1, or other loci meeting the criteria of a genome safe harbor. Additional integration sites include an endogenous gene locus intended for disruption, such as reduction or knockout, which comprises B2M, TAP1, TAP2, tapasin, NLRC5, CIITA, RFXANK, RFX5, RFXAP, TCRα or TCRβ constant region (TRAC or TRBC), NKG2A, NKG2D, CD38, CD25, CD69, CD71, CD44, CD54, CD56, CD58, OX40, 4-1BB, CIS, CBL-B, SOCS2, PD1, CTLA4, LAG3, TIM3, or TIGIT.

In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCRα or TCRβ constant region, NKG2A, NKG2D, CD38, CD25, CD69, CD71, CD44, CD54, CD56, CD58, OX40, 4-1BB, CIS, CBL-B, SOCS2, PD1, CTLA4, LAG3, TIM3, or TIGIT. In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCRα or TCRβ constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, or TIGIT. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCRα or TCRβ constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, or TIGIT. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCRα or TCRβ constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, or TIGIT.

Further, as provided herein, the above method for targeted integration in a safe harbor is used to insert any polynucleotide of interest, for example, polynucleotides encoding safety switch proteins, targeting modality, receptors, signaling molecules, transcription factors, pharmaceutically active proteins and peptides, drug target candidates, and proteins promoting engraftment, trafficking, homing, viability, self-renewal, persistence, and/or survival of stem cells and/or progenitor cells. In some other embodiments, the construct comprising one or more exogenous polynucleotides further comprises one or more marker genes. In one embodiment, the exogenous polynucleotide in a construct of the invention is a suicide gene encoding a safety switch protein. Suitable suicide gene systems for induced cell death include, but not limited to Caspase 9 (or caspase 3 or 7) and AP1903; thymidine kinase (TK) and ganciclovir (GCV); cytosine deaminase (CD) and 5-fluorocytosine (5-FC). Additionally, some suicide gene systems are cell type specific, for example, the genetic modification of T lymphocytes with the B-cell molecule CD20 allows their elimination upon administration of mAb Rituximab. Further, modified EGFR containing epitope recognized by cetuximab can be used to deplete genetically engineered cells when the cells are exposed to cetuximab. As such, one aspect of the invention provides a method of targeted integration of one or more suicide genes encoding safety switch proteins selected from caspase 9 (caspase 3 or 7), thymidine kinase, cytosine deaminase, modified EGFR, and B cell CD20.

In some embodiments, one or more exogenous polynucleotides integrated by the method described herein are driven by operatively-linked exogenous promoters comprised in the construct for targeted integration. The promoters may be inducible, or constructive, and may be temporal-, tissue- or cell type-specific. Suitable constructive promoters for methods of the invention include, but not limited to, cytomegalovirus (CMV), elongation factor 1α (EF1α), phosphoglycerate kinase (PGK), hybrid CMV enhancer/chicken β-actin (CAG) and ubiquitin C (UBC) promoters. In one embodiment, the exogenous promoter is CAG.

The exogenous polynucleotides integrated by the method described herein may be driven by endogenous promoters in the host genome, at the integration site. In one embodiment, the method described herein is used for targeted integration of one or more exogenous polynucleotides at AAVS1 locus in the genome of a cell. In one embodiment, at least one integrated polynucleotide is driven by the endogenous AAVS1 promoter. In another embodiment, the method described herein is used for targeted integration at ROSA26 locus in the genome of a cell. In one embodiment, at least one integrated polynucleotide is driven by the endogenous ROSA26 promoter. In still another embodiment, the method described herein is used for targeted integration at H11 locus in the genome of a cell. In one embodiment, at least one integrated polynucleotide is driven by the endogenous H11 promoter. In another embodiment, the method described herein is used for targeted integration at collagen locus in the genome of a cell. In one embodiment, at least one integrated polynucleotide is driven by the endogenous collagen promoter. In still another embodiment, the method described herein is used for targeted integration at HTRP locus in the genome of a cell. In one embodiment, at least one integrated polynucleotide is driven by the endogenous HTRP promoter. Theoretically, only correct insertions at the desired location would enable gene expression of an exogenous gene driven by an endogenous promoter.

In some embodiments, the one or more exogenous polynucleotides comprised in the construct for the methods of targeted integration are driven by one promoter. In some embodiments, the construct comprises one or more linker sequences between two adjacent polynucleotides driven by the same promoter to provide greater physical separation between the moieties and maximize the accessibility to enzymatic machinery. The linker peptide of the linker sequences may consist of amino acids selected to make the physical separation between the moieties (exogenous polynucleotides, and/or the protein or peptide encoded therefrom) more flexible or more rigid depending on the relevant function. The linker sequence may be cleavable by a protease or cleavable chemically to yield separate moieties. Examples of enzymatic cleavage sites in the linker include sites for cleavage by a proteolytic enzyme, such as enterokinase, Factor Xa, trypsin, collagenase, and thrombin. In some embodiments, the protease is one which is produced naturally by the host or it is exogenously introduced. Alternatively, the cleavage site in the linker may be a site capable of being cleaved upon exposure to a selected chemical, e.g., cyanogen bromide, hydroxylamine, or low pH. The optional linker sequence may serve a purpose other than the provision of a cleavage site. The linker sequence should allow effective positioning of the moiety with respect to another adjacent moiety for the moieties to function properly. The linker may also be a simple amino acid sequence of a sufficient length to prevent any steric hindrance between the moieties. In addition, the linker sequence may provide for post-translational modification including, but not limited to, e.g., phosphorylation sites, biotinylation sites, sulfation sites, γ-carboxylation sites, and the like. In some embodiments, the linker sequence is flexible so as not to hold the biologically active peptide in a single undesired conformation. The linker may be predominantly comprised of amino acids with small side chains, such as glycine, alanine, and serine, to provide for flexibility. In some embodiments about 80 to 90 percent or greater of the linker sequence comprises glycine, alanine, or serine residues, particularly glycine and serine residues. In several embodiments, a G4S linker peptide separates the end-processing and endonuclease domains of the fusion protein. In other embodiments, a 2A linker sequence allows for two separate proteins to be produced from a single translation. Suitable linker sequences can be readily identified empirically. Additionally, suitable size and sequences of linker sequences also can be determined by conventional computer modeling techniques. In one embodiment, the linker sequence encodes a self-cleaving peptide. In one embodiment, the self-cleaving peptide is 2A. In some other embodiments, the linker sequence provides an Internal Ribosome Entry Sequence (IRES). In some embodiments, any two consecutive linker sequences are different.

The method of introducing into cells a construct comprising exogenous polynucleotides for targeted integration can be achieved using a method of gene transfer to cells known per se. In one embodiment, the construct comprises backbones of viral vectors such as adenovirus vector, adeno-associated virus vector, retrovirus vector, lentivirus vector, Sendai virus vector. In some embodiments, the plasmid vectors are used for delivering and/or expressing the exogenous polynucleotides to target cells (e.g., pAl-11, pXTl, pRc/CMV, pRc/RSV, pcDNAI/Neo) and the like. In some other embodiments, the episomal vector is used to deliver the exogenous polynucleotide to target cells. In some embodiments, recombinant adeno-associated viruses (rAAV) can be used for genetic engineering to introduce insertions, deletions or substitutions through homologous recombination. Unlike lentiviruses, rAAVs do not integrate into the host genome. In addition, episomal rAAV vectors mediate homology-directed gene targeting at much higher rates compared to transfection of conventional targeting plasmids. In some embodiments, an AAV6 or AAV2 vector is used to introduce insertions, deletions or substitutions in a target site in the genome of iPSCs. In some embodiments, the genomically modified iPSCs and their derivative cells obtained using the methods and compositions described herein comprise at least one genotype listed in Table 1.

III. Method of Obtaining and Maintaining Genome-Engineered iPSCs

In various embodiments, the present invention provides a method of obtaining and maintaining genome-engineered iPSCs comprising one or more targeted edits at one or more desired sites, wherein the one or more targeted edits remain intact and functional in expanded genome-engineered iPSCs or the iPSC-derived non-pluripotent cells at the respective selected editing sites. The targeted editing introduces into the genome of the iPSC, and derivative cells thereof, insertions, deletions, and/or substitutions (i.e., targeted integration and/or in/dels at selected sites). In comparison to direct engineering of patient-sourced, peripheral blood originated primary effector cells, the many benefits of obtaining genomically-engineered derivative cells through editing and differentiating iPSC as provided herein include, but are not limited to: unlimited source for engineered effector cells; no need for repeated manipulation of the effector cells, especially when multiple engineered modalities are involved; the obtained effector cells are rejuvenated for having elongated telomere and experiencing less exhaustion; the effector cell population is homogeneous in terms of editing site, copy number, and void of allelic variation, random mutations and expression variegation, largely due to the enabled clonal selection in engineered iPSCs as provided herein.

In particular embodiments, the genome-engineered iPSCs comprising one or more targeted edits at one or more selected sites are maintained, passaged and expanded as single cells for an extended period in the cell culture medium shown in Table 2 as Fate Maintenance Medium (FM), wherein the iPSCs retain the targeted editing and functional modification at the selected site(s). The components of the medium may be present in the medium in amounts within an optimal range shown in Table 2. The iPSCs cultured in F M have been shown to continue to maintain their undifferentiated, and ground or naïve, profile; provide genomic stability without the need for culture cleaning or selection; and readily to give rise to all three somatic lineages by in vitro differentiation via embryoid bodies or monolayer (without formation of embryoid bodies); and by in vivo differentiation via teratoma formation. See, for example, International Pub. No. WO2015/134652, the disclosure of which is incorporated herein by reference.

TABLE 2
Exemplary media for iPSC reprogramming and maintenance

| Conventional hESC | Fate Reprogramming | Fate Maintenance |
|---|---|---|
| Medium (Conv.) | Medium (FRM) | Medium (FMM) |
| DMEM/F12 | DMEM/F12 | DMEM/F12 |
| Knockout Serum | Knockout Serum | Knockout Serum |
| Replacement (20%) | Replacement (20%) | Replacement (20%) |
| | N2 | |
| | B27 | |
| Glutamine | Glutamine | Glutamine (1x) |
| Non-Essential Amino | Non-Essential Amino | Non-Essential Amino |
| Acids (1x) | Acids (1x) | Acids (1x) |
| β-mercaptoethanol | β-mercaptoethanol | β-mercaptoethanol |
| (100 μM) | (100 μM) | (100 μM) |
| bFGF (0.2-50 ng/mL) | bFGF (2-500 ng/mL) | bFGF (2-500 ng/mL) |
| | LIF (0.2-50 ng/mL) | LIF (0.2-50 ng/mL) |
| | Thiazovivin (0.1-25 μM) | Thiazovivin (0.1-25 μM) |
| | PD0325901 (0.005-2 μM) | PD0325901 (0.005-2 μM) |
| | CHIR99021 (0.02-5 μM) | CHIR99021 (0.02-5 μM) |
| | SB431542 (0.04-10 μM) | |

| | |
|---|---|
| In combination with | Feeder-free, in combination with |
| MEF feeder cells | Matrigel ™ or Vitronectin |

In some embodiments, the genome-engineered iPSCs comprising one or more targeted integrations and/or in/dels are maintained, passaged and expanded in a medium comprising a MEK inhibitor, a GSK3 inhibitor, and a ROCK inhibitor, and free of, or essentially free of, TGFβ receptor/ALK5 inhibitors, wherein the iPSCs retain the intact and functional targeted edits at the selected sites.

Another aspect of the invention provides a method of generating genome-engineered iPSCs through targeted editing of iPSCs; or through first generating genome-engineered non-pluripotent cells by targeted editing, and then reprogramming the selected/isolated genome-engineered non-pluripotent cells to obtain iPSCs comprising the same targeted editing as the non-pluripotent cells. A further aspect of the invention provides genome-engineering non-pluripotent cells which are concurrently undergoing reprogramming by introducing targeted integration and/or targeted in/dels to the cells, wherein the contacted non-pluripotent cells are under sufficient conditions for reprogramming, and wherein the conditions for reprogramming comprise contacting non-pluripotent cells with one or more reprogramming factors and small molecules. In various embodiments of the method for concurrent genome-engineering and reprogramming, the targeted integrations and/or targeted in/dels may be introduced to the non-pluripotent cells prior to, or essentially concomitantly with, initiating reprogramming by contacting the non-pluripotent cells with one or more reprogramming factors and optionally one or more small molecules.

In some embodiments, to concurrently genome-engineer and reprogram non-pluripotent cells, the targeted integrations and/or in/dels may also be introduced to the non-pluripotent cells after the multi-day process of reprogramming is initiated by contacting the non-pluripotent cells with one or more reprogramming factors and small molecules, and wherein the vectors carrying the constructs are introduced before the reprogramming cells present stable expression of one or more endogenous pluripotent genes including, but not limited to, SSEA4, Tra181 and CD30.

In some embodiments, the reprogramming is initiated by contacting the non-pluripotent cells with at least one reprogramming factor, and optionally a combination of a TGFβ receptor/ALK inhibitor, a MEK inhibitor, a GSK3 inhibitor and a ROCK inhibitor (FRM; Table 2). In some embodiments, the genome-engineered iPSCs produced through any methods above are further maintained and expanded using a mixture comprising a combination of a MEK inhibitor, a GSK3 inhibitor and a ROCK inhibitor (FMM; Table 2).

In some embodiments of the method of generating genome-engineered iPSCs, the method comprises: genomically engineering an iPSC by introducing one or more targeted integrations and/or in/dels into iPSCs to obtain genome-engineered iPSCs having at least one genotype listed in Table 1. Alternatively, the method of generating genome-engineered iPSCs comprises: (a) introducing one or more targeted edits into non-pluripotent cells to obtain genome-engineered non-pluripotent cells comprising targeted integrations and/or in/dels at selected sites, and (b) contacting the genome-engineered non-pluripotent cells with one or more reprogramming factors, and optionally a small molecule composition comprising a TGFβ receptor/ALK inhibitor, a MEK inhibitor, a GSK3 inhibitor and/or a ROCK inhibitor, to obtain genome-engineered iPSCs comprising targeted integrations and/or in/dels at selected sites. Alternatively, the method of generating genome-engineered iPSCs comprises: (a) contacting non-pluripotent cells with one or more reprogramming factors, and optionally a small molecule composition comprising a TGFβ receptor/ALK inhibitor, a MEK inhibitor, a GSK3 inhibitor and/or a ROCK inhibitor to initiate the reprogramming of the non-pluripotent cells; (b) introducing one or more targeted integrations and/or in/dels into the reprogramming non-pluripotent cells for genome-engineering; and (c) obtaining clonal genome-engineered iPSCs comprising the targeted integrations and/or in/dels at selected sites. Any of the above methods may further comprise single cell sorting of the genome-engineered iPSCs to obtain a clonal iPSC. Through clonal expansion of the genome-engineered iPSCs, a master cell bank is generated to comprise single cell sorted and expanded clonal engineered iPSCs having at least one phenotype as provided herein. The master cell bank is subsequently cryopreserved, providing a platform for additional iPSC engineering and a renewable source for manufacturing off-the-shelf, engineered, homogeneous cell therapy products, which are well-defined and uniform in composition, and can be mass produced at significant scale in a cost-effective manner.

The reprogramming factors are selected from the group consisting of OCT4, SOX2, NANOG, KLF4, LIN28, C-MYC, ECAT1, UTF1, ESRRB, SV40LT, HESRG, CDH1, TDGF1, DPPA4, DNMT3B, ZIC3, L1TD1, and any combinations thereof as disclosed in International Pub. Nos. WO2015/134652 and WO 2017/066634, the disclosures of which are incorporated herein by reference. The one or more reprogramming factors may be in the form of a polypeptide. The reprogramming factors may also be in the form of polynucleotides encoding the reprogramming factors, and thus may be introduced to the non-pluripotent cells by vectors such as, a retrovirus, a Sendai virus, an adenovirus, an episome, a plasmid, and a mini-circle. In particular embodiments, the one or more polynucleotides encoding at least one reprogramming factor are introduced by a lentiviral vector. In some embodiments, the one or more polynucleotides introduced by an episomal vector. In various other embodiments, the one or more polynucleotides are introduced by a Sendai viral vector. In some embodiments, the one or more polynucleotides introduced by a combination of plasmids. See, for example, International Pub. No. WO2019/075057A1, the disclosure of which is incorporated herein by reference.

In some embodiments, the non-pluripotent cells are transfected with multiple constructs comprising different exogenous polynucleotides and/or different promoters by multiple vectors for targeted integration at the same or different selected sites. These exogenous polynucleotides may comprise a suicide gene, or gene encoding targeting modalities, receptors, signaling molecules, transcription factors, pharmaceutically active proteins and peptides, drug target candidates, or a gene encoding a protein promoting engraftment, trafficking, homing, viability, self-renewal, persistence, and/or survival of the iPSCs or derivative cells thereof. In some embodiments, the exogenous polynucleotides encode RNA, including but not limited to siRNA, shRNA, miRNA and antisense nucleic acids. These exogenous polynucleotides may be driven by one or more promoters selected from the group consisting of constitutive promoters, inducible promoters, temporal-specific promoters, and tissue or cell type specific promoters. Accordingly, the polynucleotides are expressible when under conditions that activate the promoter, for example, in the presence of an inducing agent or in a particular differentiated cell type. In some embodiments, the polynucleotides are expressed in iPSCs and/or in cells differentiated from the iPSCs. In one embodiment, one or more suicide genes are driven by a constitutive promoter, for example Caspase-9 driven by CAG. These constructs comprising different exogenous polynucleotides and/or different promoters can be transfected to non-pluripotent cells either simultaneously or consecutively. The non-pluripotent cells subjected to targeted integration of multiple constructs can simultaneously contact the one or more reprogramming factors to initiate the reprogramming concurrently with the genomic engineering, thereby obtaining genome-engineered iPSCs comprising multiple targeted integrations in the same pool of cells. As such, this robust method enables a concurrent reprogramming and engineering strategy to derive a clonal genomically-engineered iPSC with multiple modalities integrated into one or more selected target sites. In some embodiments, the genomically modified iPSCs and their derivative cells obtained using the methods and composition herein comprise at least one genotype listed in Table 1.

IV. A Method of Obtaining Genetically-Engineered Effector Cells by Differentiating Genome-Engineered iPSC

A further aspect of the present invention provides a method of in vivo differentiation of genome-engineered iPSCs by teratoma formation, wherein the differentiated cells derived in vivo from the genome-engineered iPSCs retain the intact and functional targeted edits including targeted integration(s) and/or in/dels at the desired site(s). In some embodiments, the differentiated cells derived in vivo from the genome-engineered iPSCs via teratoma formation comprise one or more inducible suicide genes integrated at one or more desired sites comprising AAVS1, CCR5, ROSA26, collagen, HTRP H11, beta-2 microglobulin, CD38, GAPDH, TCR or RUTNX1, or other loci meeting the criteria of a genome safe harbor. In some other embodiments, the differentiated cells derived in vivo from the genome-engineered iPSCs via teratoma formation comprise polynucleotides encoding targeting modalities, or encoding proteins promoting trafficking, homing, viability, self-renewal, persistence, and/or survival of stem cells and/or progenitor cells. In some embodiments, the differentiated cells derived in vivo from the genome-engineered iPSCs via teratoma formation comprising one or more inducible suicide genes further comprise one or more in/dels in endogenous genes associated with immune response regulation and mediation. In some embodiments, the in/del is comprised in one or more endogenous checkpoint genes. In some embodiments, the in/del is comprised in one or more endogenous T cell receptor genes. In some embodiments, the in/del is comprised in one or more endogenous MHC class I suppressor genes. In some embodiments, the in/del is comprised in one or more endogenous genes associated with the major histocompatibility complex. In some embodiments, the in/del is comprised in one or more endogenous genes including, but not limited to, AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCRα or TCRβ constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, and TIGIT. In one embodiment, the genome-engineered iPSCs comprising one or more exogenous polynucleotides at selected site(s) further comprise a targeted edit in a B2M (beta-2-microglobulin) encoding gene.

In particular embodiments, the genome-engineered iPSCs comprising one or more genetic modifications as provided herein are used to derive hematopoietic cell lineages or any other specific cell types in vitro, wherein the derived non-pluripotent cells retain the functional genetic modifications including targeted editing at the selected site(s). In one embodiment, the genome-engineered iPSC-derived cells include, but are not limited to, mesodermal cells with definitive hemogenic endothelium (HE) potential, definitive HE, CD34 hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, B cells, neutrophils, dendritic cells, and macrophages, wherein the cells derived from the genome-engineered iPSCs retain the functional genetic modifications including targeted editing at the desired site(s).

Applicable differentiation methods and compositions for obtaining iPSC-derived hematopoietic cell lineages include those depicted in, for example, International Pub. No. WO2017/078807, the disclosure of which is incorporated herein by reference. As provided, the methods and compositions for generating hematopoietic cell lineages are through definitive hemogenic endothelium (HE) derived from pluripotent stem cells, including iPSCs under serum-free, feeder-free, and/or stromal-free conditions and in a scalable and monolayer culturing platform without the need of EB formation. Cells that may be differentiated according to the provided methods range from pluripotent stem cells, to progenitor cells that are committed to particular terminally differentiated cells and transdifferentiated cells, and to cells of various lineages directly transitioned to hematopoietic fate without going through a pluripotent intermediate. Similarly, the cells that are produced by differentiating stem cells range from multipotent stem or progenitor cells, to terminally differentiated cells, and to all intervening hematopoietic cell lineages.

The methods for differentiating and expanding cells of the hematopoietic lineage from pluripotent stem cells in monolayer culturing comprise contacting the pluripotent stem cells with a BMP pathway activator, and optionally, bFGF. As provided, the pluripotent stem cell-derived mesodermal cells are obtained and expanded without forming embryoid bodies from pluripotent stem cells. The mesodermal cells are then subjected to contact with a BMP pathway activator, bFGF, and a WNT pathway activator to obtain expanded mesodermal cells having definitive hemogenic endothelium (HE) potential without forming embryoid bodies from the pluripotent stem cells. By subsequent contact with bFGF, and optionally, a ROCK inhibitor, and/or a WNT pathway activator, the mesodermal cells having definitive HE potential are differentiated to definitive HE cells, which are also expanded during differentiation.

The methods provided herein for obtaining cells of the hematopoietic lineage are superior to EB-mediated pluripotent stem cell differentiation, because EB formation leads to modest to minimal cell expansion, does not allow monolayer culturing which is important for many applications requiring homogeneous expansion and homogeneous differentiation of the cells in a population, and is laborious and of low efficiency.

The provided monolayer differentiation platform facilitates differentiation towards definitive hemogenic endothelium resulting in the derivation of hematopoietic stem cells and differentiated progeny such as T, B, NKT and NK cells. The monolayer differentiation strategy combines enhanced differentiation efficiency with large-scale expansion, and enables the delivery of a therapeutically relevant number of pluripotent stem cell-derived hematopoietic cells for various therapeutic applications. Further, monolayer culturing using the methods provided herein leads to functional hematopoietic lineage cells that enable a full range of in vitro differentiation, ex vivo modulation, and in vivo long term hematopoietic self-renewal, reconstitution and engraftment. As provided, the iPSC-derived hematopoietic lineage cells include, but are not limited to, definitive hemogenic endothelium, hematopoietic multipotent progenitor cells, hematopoietic stem and progenitor cells, T cell progenitors, NK cell progenitors, T cells, NK cells, NKT cells, B cells, macrophages, and neutrophils.

Thus, in various embodiments, the method for directing differentiation of pluripotent stem cells into cells of a definitive hematopoietic lineage, comprises: (i) contacting pluripotent stem cells with a composition comprising a BMP activator, and optionally bFGF, to initiate differentiation and expansion of mesodermal cells from the pluripotent stem cells; (ii) contacting the mesodermal cells with a composition comprising a BMP activator, bFGF, and a GSK3 inhibitor, wherein the composition is optionally free of TGFβ receptor/ALK inhibitor, to initiate differentiation and expansion of mesodermal cells having definitive HE potential from the mesodermal cells; (iii) contacting the mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator, wherein the composition is optionally free of TGFβ receptor/ALK inhibitor, to initiate differentiation and expansion of definitive hemogenic endothelium from pluripotent stem cell-derived mesodermal cells having definitive hemogenic endothelium potential.

In some embodiments, the method further comprises contacting pluripotent stem cells with a composition comprising a MEK inhibitor, a GSK3 inhibitor, and a ROCK inhibitor, wherein the composition is free of TGFβ receptor/ALK inhibitors, to seed and expand the pluripotent stem cells. In some embodiments, the pluripotent stem cells are iPSCs, or naïve iPSCs, or iPSCs comprising one or more genetic imprints; and the one or more genetic imprints comprised in the iPSCs are retained in the hematopoietic cells differentiated therefrom. In some embodiments of the method for directing differentiation of pluripotent stem cells into cells of a hematopoietic lineage, the differentiation of the pluripotent stem cells into cells of hematopoietic lineage is void of generation of embryoid bodies and is in a monolayer culturing form.

In some embodiments of the above method, the obtained pluripotent stem cell-derived definitive hemogenic endothelium cells are CD34⁺. In some embodiments, the obtained definitive hemogenic endothelium cells are CD34⁺CD43⁻. In some embodiments, the definitive hemogenic endothelium cells are CD34⁺CD43⁻CXCR4⁻CD73⁻. In some embodiments, the definitive hemogenic endothelium cells are CD34⁺CXCR4⁻CD73⁻. In some embodiments, the definitive hemogenic endothelium cells are CD34⁺CD43⁻CD93⁻. In some embodiments, the definitive hemogenic endothelium cells are CD34⁺CD93⁻.

In some embodiments of the above method, the method further comprises (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, and IL7; and optionally a BMP activator; to initiate the differentiation of the definitive hemogenic endothelium to pre-T cell progenitors; and optionally, (ii) contacting the pre-T cell progenitors with a composition comprising one or more growth factors and cytokines selected from the group consisting of SCF, Flt3L, and IL7, but free of one or more of VEGF, bFGF, TPO, BMP activators and ROCK inhibitors, to initiate the differentiation of the pre-T cell progenitors to T cell progenitors or T cells. In some embodiments of the method, the pluripotent stem cell-derived T cell progenitors are CD34⁺CD45⁺CD7⁺. In some embodiments of the method, the pluripotent stem cell-derived T cell progenitors are CD45⁺CD7⁺.

In yet some embodiments of the above method for directing differentiation of pluripotent stem cells into cells of a hematopoietic lineage, the method further comprises: (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, IL3, IL7, and IL15; and optionally, a BMP activator, to initiate differentiation of the definitive hemogenic endothelium to pre-NK cell progenitor; and optionally, (ii) contacting pluripotent stem cells-derived pre-NK cell progenitors with a composition comprising one or more growth factors and cytokines selected from the group consisting of SCF, Flt3L, IL3, IL7, and IL15, wherein the medium is free of one or more of VEGF, bFGF, TPO, BMP activators and ROCK inhibitors, to initiate differentiation of the pre-NK cell progenitors to NK cell progenitors or NK cells. In some embodiments, the pluripotent stem cell-derived NK progenitors are CD3⁻CD45⁺CD56⁺CD7⁺. In some embodiments, the pluripotent stem cell-derived NK cells are CD3⁻CD45⁺CD56⁺, and optionally further defined by being NKp46⁺, CD57⁺ and CD16⁺.

Therefore, using the above differentiation methods, one may obtain one or more populations of iPSC-derived hematopoietic cells: (i) CD34⁺ HE cells (iCD34), using one or more culture medium selected from iMPP-A, iTC-A2, iTC-B2, iNK-A2, and iNK-B2; (ii) definitive hemogenic endothelium (iHE), using one or more culture medium selected from iMPP-A, iTC-A2, iTC-B2, iNK-A2, and iNK-B2; (iii) definitive HSCs, using one or more culture medium selected from iMPP-A, iTC-A2, iTC-B2, iNK-A2, and iNK-B2; (iv) multipotent progenitor cells (iMPP), using iMPP-A; (v) T cell progenitors (ipro-T), using one or more culture medium selected from iTC-A2, and iTC-B2; (vi) T cells (iTC), using iTC-B2; (vii) NK cell progenitors (ipro-NK), using one or more culture medium selected from iNK-A2, and iNK-B2; and/or (viii) NK cells (iNK), and iNK-B2. In some embodiments, the medium:
a. iCD34-C comprises a ROCK inhibitor, one or more growth factors and cytokines selected from the group consisting of bFGF, VEGF, SCF, IL6, IL11, IGF, and EPO, and optionally, a Wnt pathway activator; and is free of TGFβ receptor/ALK inhibitor;
b. iMPP-A comprises a BMP activator, a ROCK inhibitor, and one or more growth factors and cytokines selected from the group consisting of TPO, IL3, GMCSF, EPO, bFGF, VEGF, SCF, IL6, Flt3L and IL11;
c. iTC-A2 comprises a ROCK inhibitor; one or more growth factors and cytokines selected from the group consisting of SCF, Flt3L, TPO, and IL7; and optionally, a BMP activator;
d. iTC-B2 comprises one or more growth factors and cytokines selected from the group consisting of SCF, Flt3L, and IL7;
e. iNK-A2 comprises a ROCK inhibitor, and one or more growth factors and cytokines selected from the group consisting of SCF, Flt3L, TPO, IL3, IL7, and IL15; and optionally, a BMP activator, and
f. iNK-B2 comprises one or more growth factors and cytokines selected from the group consisting of SCF, Flt3L, IL7 and IL15.

In some embodiments, the genome-engineered iPSC-derived cells obtained from the above methods comprise one or more inducible suicide genes integrated at one or more desired integration sites comprising AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCRα or TCRβ constant region, NKG2A, NKG2D, CD25, CD38, CD44, CD54, CD56, CD58, CD69, CD71, OX40, 4-1BB, CIS, CBL-B, SOCS2, PD1, CTLA4, LAG3, TIM3, and TIGIT, or other loci meeting the criteria of a genome safe harbor. In some other embodiments, the genome-engineered iPSC-derived cells comprise polynucleotides encoding safety switch proteins, targeting modality, receptors, signaling molecules, transcription factors, pharmaceutically active proteins and peptides, drug target candidates, or proteins promoting trafficking, homing, viability, self-renewal, persistence, and/or survival of stem cells and/or progenitor cells. In some embodiments, the genome-engineered iPSC-derived cells comprising one or more suicide genes further comprise one or more in/dels comprised in one or more endogenous genes associated with immune response regulation and mediation, including, but not limited to, checkpoint genes, endogenous T cell receptor genes, and MHC class I suppressor genes.

Additionally, applicable dedifferentiation methods and compositions for obtaining genomic-engineered hematopoietic cells of a first fate to genomic-engineered hematopoietic cells of a second fate include those depicted in, for example, International Pub. No. WO2011/159726, the disclosure of which is incorporated herein by reference. The method and composition provided therein allows partially reprogramming a starting non-pluripotent cell to a non-pluripotent intermediate cell by limiting the expression of endogenous Nanog gene during reprogramming; and subjecting the non-pluripotent intermediate cell to conditions for differentiating the intermediate cell into a desired cell type. In some embodiments, the genomically modified iPSCs and their derivative cells obtained using the methods and compositions described herein comprise at least one genotype listed in Table 1.

V. Therapeutic Use of Derivative Immune Cells with Functional Modalities Differentiated from Genetically Engineered iPSCs

The present invention provides, in some embodiments, a composition comprising an isolated population or subpopulation of functionally enhanced derivative immune cells that have been differentiated from genomically engineered iPSCs using the methods and compositions as disclosed. In some embodiments, the iPSCs comprise one or more targeted genetic edits which are retainable in the iPSC-derived effector cells, wherein the genetically engineered iPSCs and derivative cells thereof are suitable for cell-based adoptive therapies. In one embodiment, the isolated population or subpopulation of genetically engineered effector cells comprises iPSC-derived CD34 cells. In one embodiment, the isolated population or subpopulation of genetically engineered effector cells comprises iPSC-derived HSC cells. In one embodiment, the isolated population or subpopulation of genetically engineered effector cells comprises iPSC-derived proT or T cells. In one embodiment, the isolated population or subpopulation of genetically engineered effector cells comprises iPSC-derived proNK or NK cells. In one embodiment, the isolated population or subpopulation of genetically engineered effector cells comprises iPSC-derived immune regulatory cells or myeloid derived suppressor cells (MDSCs). In some embodiments, the iPSC-derived genetically engineered effector cells are further modulated ex vivo for improved therapeutic potential. In one embodiment, an isolated population or subpopulation of genetically engineered effector cells that have been derived from iPSCs comprises an increased number or ratio of naïve T cells, stem cell memory T cells, and/or central memory T cells. In one embodiment, the isolated population or subpopulation of genetically engineered effector cells that have been derived from iPSCs comprises an increased number or ratio of type I NKT cells. In another embodiment, the isolated population or subpopulation of genetically engineered effector cells that have been derived from iPSCs comprises an increased number or ratio of adaptive NK cells. In some embodiments, the isolated population or subpopulation of genetically engineered CD34 cells, HSC cells, T cells, NK cells, or myeloid derived suppressor cells derived from iPSCs are allogeneic. In some other embodiments, the isolated population or subpopulation of genetically engineered CD34 cells, HSC cells, T cells, NK cells, or MDSCs derived from iPSC are autologous.

In some embodiments, the iPSC for differentiation comprises genetic imprints selected to convey desirable therapeutic attributes in derived effector cells, provided that cell development biology during differentiation is not disrupted, and provided that the genetic imprints are retained and functional in the differentiated hematopoietic cells derived from said iPSC.

In some embodiments, the genetic imprints of the pluripotent stem cells comprise (i) one or more genetically modified modalities obtained through genomic insertion, deletion or substitution in the genome of the pluripotent cells during or after reprogramming a non-pluripotent cell to iPSC; or (ii) one or more retainable therapeutic attributes of a source specific immune cell that is donor-, disease-, or treatment response-specific, and wherein the pluripotent cells are reprogrammed from the source specific immune cell, wherein the iPSC retain the source therapeutic attributes, which are also comprised in the iPSC-derived hematopoietic lineage cells.

In some embodiments, the genetically modified modalities comprise one or more of: safety switch proteins, targeting modalities, receptors, signaling molecules, transcription factors, pharmaceutically active proteins and peptides, drug target candidates; or proteins promoting engraftment, trafficking, homing, viability, self-renewal, persistence, immune response regulation and modulation, and/or survival of the iPSCs or derivative cells thereof. In some embodiments, the genetically modified iPSC and the derivative cells thereof comprise a genotype listed in Table 1. In some other embodiments, the genetically modified iPSC and the derivative cells thereof comprising a genotype listed in Table 1 further comprise additional genetically modified modalities comprising (1) one or more of deletion or disruption of B2M, TAP1, TAP2, tapasin, NLRC5, CIITA, RFXANK, RFX5, RFXAP, TCRα or TCRβ constant region (TRAC or TRBC), NKG2A, NKG2D, CD38, CD25, CD69, CD71, CD44, CD54, CD56, CD58, OX40, 4-1BB, CIS, CBL-B, SOCS2, PD1, CTLA4, LAG3, TIM3, or TIGIT; and/or (2) introduction of HLA-E, HLA-G, 4-1BBL, CD3, CD4, CD8, CD47, CD113, CD131, CD137, CD80, PDL1, A_{2A}R, CAR, TCR, Fc receptor, or surface triggering receptors for coupling with bi- or multi-specific or universal engagers.

In still some other embodiments, the iPSC-derived hematopoietic lineage cells comprise the therapeutic attributes of the source specific immune cell relating to a combination of at least two of the following: (i) expression of one or more antigen targeting receptors; (ii) modified HLA; (iii) resistance to tumor microenvironment; (iv) recruitment of bystander immune cells and immune modulations; (v) improved on-target specificity with reduced off-tumor effect; (vi) overcoming or reducing tumor microenvironment suppression associated with solid tumors; (vii) improved homing, persistence, cytotoxicity, or antigen escape rescue; and (viii) improved apoptosis resistance and/or exhaustion resistance.

In some embodiments, the iPSC-derived hematopoietic cells comprise a genotype listed in Table 1, and said cells express a Fas redirector (“Fas” in Table 1), wherein the Fas redirector comprises (a) an extracellular Fas binding domain comprising a partial or full peptide of an extracellular domain (ECD) of a Fas receptor (FAS), or a variant or an allele thereof, and (b) a cytoplasmic signaling domain comprising a partial or full peptide of an intracellular domain (ICD) of one or more co-stimulation molecules, wherein the Fas redirector redirects Fas signaling upon binding to a FAS agonist, thereby providing the cell with improved apoptosis resistance and/or exhaustion resistance. In various embodiments, the Fas redirector further comprises a transmembrane region comprising a transmembrane domain, or a portion thereof, of a transmembrane protein. In some embodiments, the one or more co-stimulation molecules comprises CD27, CD28, CD40, MyD88, OX40, IL12Rβb2, IL18R1, IL21R, or a combination thereof. In some embodiments, the one or more co-stimulation molecules does not comprise 41BB. In some embodiments, the FAS agonist comprises a Fas ligand (FasL). In some embodiments, the cell comprising the Fas redirector further comprises one or more of: (a) an exogenous polynucleotide encoding a chimeric antigen receptor (CAR); (b) an exogenous polynucleotide encoding a CD16 or a variant thereof, (c) CD38 knockout; and (d) an exogenous polynucleotide encoding a cytokine signaling complex comprising a partial or full peptide of a cell surface expressed exogenous cytokine and/or a receptor thereof.

Specifically, the present application provides a method of improving efficacy of an adoptive cell therapy provided to a subject in need thereof, wherein the method comprises administering the derivative effector cells or a population thereof, as described herein. In various embodiments, the derivative effector cells comprise a Fas redirector, wherein the Fas redirector comprises (a) an extracellular Fas binding domain comprising a partial or full peptide of an extracellular domain (ECD) of a Fas receptor (FAS), or a variant or an allele thereof, and (b) a cytoplasmic signaling domain comprising a partial or full peptide of an intracellular domain (ICD) of one or more co-stimulation molecules, and wherein the Fas redirector redirects Fas signaling upon binding to a FAS agonist, thereby providing the cell with improved apoptosis resistance and/or exhaustion resistance. In various embodiments, the derived effector cells optionally further comprise one or more of a CAR, exogenous CD16, CD38^{-/-}, a cytokine signaling complex, HLA deficiency/modification, an antibody, a checkpoint inhibitor, an engager, and any other modality, as shown in Table 1. In various embodiments, the transmembrane region of the Fas redirector comprises (i) a full length or at least a portion of the native or modified transmembrane region of FAS, CD2, CD3δ, CD3ε, CD3γ, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide; or (ii) a full or partial length of the transmembrane domain of FAS. In certain embodiments, (i) the extracellular binding domain of the Fas redirector comprises a sequence having at least about 85%, about 90%, about 95%, about 96%, about 97%, about 98%, or about 99% identity to SEQ ID NO: 1; (ii) the cytoplasmic signaling domain of the Fas redirector comprises: (a) a full or partial length of the intracellular domain (ICD) of MyD88 and CD40, represented by SEQ ID NO: 2; or (b) a full or partial length of the intracellular domain (ICD) of CD27, represented by SEQ ID NO: 3; or (c) a full or partial length of the intracellular domain (ICD) of CD28, represented by SEQ ID NO: 4; or (d) a full or partial length of the intracellular domain (ICD) of OX40, represented by SEQ ID NO: 5; or (e) a full or partial length of the intracellular domain (ICD) of IL12Rβ2, represented by SEQ ID NO: 6; or (f) a full or partial length of the intracellular domain (ICD) of IL18R1, represented by SEQ ID NO: 7; or (g) a full or partial length of the intracellular domain (ICD) of IL21R, represented by SEQ ID NO: 8; or (iii) the Fas redirector comprises an amino acid sequence that has at least about 85%, about 90%, about 95%, about 96%, about 97%, about 98%, or about 99% identity to any one of SEQ ID NOs: 9-15.

In various embodiments of the method of improving efficacy of an adoptive cell therapy, the effector cells are administered to the subject as a combination therapy with a therapeutic agent. In some embodiments, the therapeutic agent of the combination therapy is an anti-CD38 antibody or fragment thereof. In some embodiments, the anti-CD38 antibody is daratumumab, isatuximab, or MOR202. In some embodiments, the anti-CD38 therapeutic agent is administered with, before, or after the administering of the derivative effector cells. Thus, in some embodiments, the antibody is used in combination with a population of the effector cells described herein by concurrent or consecutive administration to a subject. In other embodiments, such antibody or a fragment thereof may be expressed by the effector cells by genetically engineering an iPSC using an exogenous polynucleotide sequence encoding said antibody or fragment thereof and directing differentiation of the engineered iPSC, as described herein. In some embodiments of the method, the effector cells are iPSC-derived hematopoietic cells. In some embodiments of the method, the effector cells are iPSC-derived T, NK, or NKT cells.

In a further embodiment of the method of improving efficacy of an adoptive cell therapy provided to a subject in need thereof, the method further comprises administering an antibody specific to a same or different upregulated surface protein as targeted by the CAR, and/or one or more additional therapeutic agents. In some embodiments of the method, the antibody comprises at least one of an anti-CD20, an anti-HER2, an anti-CD52, an anti-EGFR, an anti-CD123, an anti-GD2, an anti-PDL1, an anti-CD38 antibody, or any of the humanized or Fc modified variants or fragments, functional equivalents and biosimilars thereof. In some embodiments of the therapeutic agents used in the method, the therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD).

A variety of diseases may be ameliorated by introducing the derivative effector cells of the invention to a subject suitable for adoptive cell therapy. In some embodiments, the iPSC-derived hematopoietic cells as provided herein are for allogeneic adoptive cell therapies. In other embodiments, the iPSC-derived hematopoietic cells as provided herein are for preventing or reducing tumor microenvironment suppression associated with solid tumors. In other embodiments, the iPSC-derived hematopoietic cells as provided herein are for improving efficacy of an adoptive cell therapy. Additionally, the present invention provides, in some embodiments, therapeutic use of the above therapeutic compositions and/or combination therapies by introducing the composition to a subject suitable for adoptive cell therapy, wherein the subject has an autoimmune disorder; a hematological malignancy; a solid tumor; or an infection associated with HIV, RSV, EBV, CMV, adenovirus, or BK polyomavirus.

Examples of hematological malignancies include, but are not limited to, acute and chronic leukemias (acute myelogenous leukemia (AML), acute lymphoblastic leukemia (ALL), chronic myelogenous leukemia (CML), lymphomas, non-Hodgkin lymphoma (NHL), Hodgkin's disease, multiple myeloma, and myelodysplastic syndromes. Examples of solid cancers include, but are not limited to, cancer of the brain, prostate, breast, lung, colon, uterus, skin, liver, bone, pancreas, ovary, testes, bladder, kidney, head, neck, stomach, cervix, rectum, larynx, and esophagus. Examples of various autoimmune disorders include, but are not limited to, alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barre syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus, erythematosus, some forms of thyroiditis, some forms of uveitis, vitiligo, granulomatosis with polyangiitis (Wegener's). Examples of viral infections include, but are not limited to, HIV—(human immunodeficiency virus), HSV—(herpes simplex virus), KSHV—(Kaposi's sarcoma-associated herpesvirus), RSV—(Respiratory Syncytial Virus), EBV—(Epstein-Barr virus), CMV—(cytomegalovirus), VZV (Varicella zoster virus), adenovirus-, a lentivirus-, a BK polyomavirus-associated disorders.

The treatment using the derived hematopoietic lineage cells of embodiments disclosed herein could be carried out upon symptom presentation, or for relapse prevention. The terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any intervention of a disease in a subject and includes: preventing the disease from occurring in a subject which may be predisposed to the disease but has not yet been diagnosed as having it; inhibiting the disease, i.e., arresting its development; or relieving the disease, i.e., causing regression of the disease. The therapeutic agent or composition may be administered before, during or after the onset of a disease or an injury. Treatment of ongoing disease, where the treatment stabilizes or reduces the undesirable clinical symptoms of the patient, is also of particular interest. In particular embodiments, the subject in need of a treatment has a disease, a condition, and/or an injury that can be contained, ameliorated, and/or improved in at least one associated symptom by a cell therapy. Certain embodiments contemplate that a subject in need of cell therapy, includes, but is not limited to, a candidate for bone marrow or stem cell transplantation, a subject who has received chemotherapy or irradiation therapy, a subject who has or is at risk of having a hyperproliferative disorder or a cancer, e.g., a hyperproliferative disorder or a cancer of hematopoietic system, a subject having or at risk of developing a tumor, e.g., a solid tumor, a subject who has or is at risk of having a viral infection or a disease associated with a viral infection.

When evaluating responsiveness to the treatment comprising the derived hematopoietic lineage cells of embodiments disclosed herein, the response can be measured by criteria comprising at least one of: clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST (Response Evaluation Criteria In Solid Tumors) criteria.

The therapeutic composition comprising iPSC-derived hematopoietic lineage cells as disclosed herein can be administered to a subject before, during, and/or after other treatments. As such a method of a combinational therapy can involve the administration or preparation of iPSC-derived effector cells before, during, and/or after the use of an additional therapeutic agent. As provided above, the one or more additional therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). The administration of the iPSC-derived immune cells can be separated in time from the administration of an additional therapeutic agent by hours, days, or even weeks. Additionally, or alternatively, the administration can be combined with other biologically active agents or modalities such as, but not limited to, an antineoplastic agent, a non-drug therapy, such as, surgery.

In some embodiments of a combinational cell therapy, the therapeutic combination comprises the iPSC-derived hematopoietic lineage cells provided herein and an additional therapeutic agent that is an antibody, or an antibody fragment. In some embodiments, the antibody is a monoclonal antibody. In some embodiments, the antibody may be a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC-derived hematopoietic lineage cells to enhance their killing ability. In some embodiments, the antibodies suitable for combinational treatment as an additional therapeutic agent to the administered iPSC-derived hematopoietic lineage cells include, but are not limited to, anti-CD20 (e.g., rituximab, veltuzumab, ofatumumab, ublituximab, ocaratuzumab, obinutuzumab), anti-HER2 (e.g., trastuzumab, pertuzumab), anti-CD52 (e.g., alemtuzumab), anti-EGFR (e.g., cetuximab), anti-GD2 (e.g., dinutuximab), anti-PDL1 (e.g., avelumab), anti-CD38 (e.g., daratumumab, isatuximab, MOR202), anti-CD123 (e.g., 7G3, CSL362), anti-SLAMF7 (elotuzumab), and their humanized or Fc modified variants or fragments or their functional equivalents or biosimilars.

In some embodiments, the additional therapeutic agent comprises one or more checkpoint inhibitors. Checkpoints are referred to cell molecules, often cell surface molecules, capable of suppressing or downregulating immune responses when not inhibited. Checkpoint inhibitors are antagonists capable of reducing checkpoint gene expression or gene products, or deceasing activity of checkpoint molecules. Suitable checkpoint inhibitors for combination therapy with the derivative effector cells, including NK or T cells, are provided above.

Some embodiments of the combination therapy comprising the provided derivative effector cells further comprise at least one inhibitor targeting a checkpoint molecule. Some other embodiments of the combination therapy with the provided derivative effector cells comprise two, three or more inhibitors such that two, three, or more checkpoint molecules are targeted. In some embodiments, the effector cells for combination therapy as described herein are derivative NK cells as provided. In some embodiments, the effector cells for combination therapy as described herein are derivative T cells. In some embodiments, the derivative NK or T cells for combination therapies are functionally enhanced as provided herein. In some embodiments, the two, three or more checkpoint inhibitors may be administered in a combination therapy with, before, or after the administering of the derivative effector cells. In some embodiments, the two or more checkpoint inhibitors are administered at the same time, or one at a time (sequential).

In some embodiments, the antagonist inhibiting any of the above checkpoint molecules is an antibody. In some embodiments, the checkpoint inhibitory antibodies may be murine antibodies, human antibodies, humanized antibodies, a camel Ig, a shark heavy-chain-only antibody (VNAR), Ig NAR, chimeric antibodies, recombinant antibodies, or antibody fragments thereof. Non-limiting examples of antibody fragments include Fab, Fab′, F(ab′)2, F(ab′)3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab, avelumab, durvalumab, ipilimumab, IPH4102, IPH43, IPH33, lirimumab, monalizumab, nivolumab, pembrolizumab, and their derivatives or functional equivalents.

The combination therapies comprising the derivative effector cells and one or more check inhibitors are applicable to treatment of liquid and solid cancers, including but not limited to cutaneous T-cell lymphoma, non-Hodgkin lymphoma (NHL), Mycosis fungoides, Pagetoid reticulosis, Sezary syndrome, Granulomatous slack skin, Lymphomatoid papulosis, Pityriasis lichenoides chronica, Pityriasis lichenoides et varioliformis acuta, CD30⁺ cutaneous T-cell lymphoma, Secondary cutaneous CD30⁺ large cell lymphoma, non-mycosis fungoides CD30 cutaneous large T-cell lymphoma, Pleomorphic T-cell lymphoma, Lennert lymphoma, subcutaneous T-cell lymphoma, angiocentric lymphoma, blastic NK-cell lymphoma, B-cell Lymphomas, hodgkins lymphoma (HL), Head and neck tumor; Squamous cell carcinoma, rhabdomyocarcoma, Lewis lung carcinoma (LLC), non-small cell lung cancer, esophageal squamous cell carcinoma, esophageal adenocarcinoma, renal cell carcinoma (RCC), colorectal cancer (CRC), acute myeloid leukemia (AML), breast cancer, gastric cancer, prostatic small cell neuroendocrine carcinoma (SCNC), liver cancer, glioblastoma, liver cancer, oral squamous cell carcinoma, pancreatic cancer, thyroid papillary cancer, intrahepatic cholangiocellular carcinoma, hepatocellular carcinoma, bone cancer, metastasis, and nasopharyngeal carcinoma.

In some embodiments, other than the derivative effector cells as provided herein, a combination for therapeutic use comprises one or more additional therapeutic agents comprising a chemotherapeutic agent or a radioactive moiety. Chemotherapeutic agent refers to cytotoxic antineoplastic agents, that is, chemical agents which preferentially kill neoplastic cells or disrupt the cell cycle of rapidly proliferating cells, or which are found to eradicate stem cancer cells, and which are used therapeutically to prevent or reduce the growth of neoplastic cells. Chemotherapeutic agents are also sometimes referred to as antineoplastic or cytotoxic drugs or agents, and are well known in the art.

In some embodiments, the chemotherapeutic agent comprises an anthracycline, an alkylating agent, an alkyl sulfonate, an aziridine, an ethylenimine, a methylmelamine, a nitrogen mustard, a nitrosourea, an antibiotic, an antimetabolite, a folic acid analog, a purine analog, a pyrimidine analog, an enzyme, a podophyllotoxin, a platinum-containing agent, an interferon, and an interleukin. Exemplary chemotherapeutic agents include, but are not limited to, alkylating agents (cyclophosphamide, mechlorethamine, mephalin, chlorambucil, heamethylmelamine, thiotepa, busulfan, carmustine, lomustine, semustine), animetabolites (methotrexate, fluorouracil, floxuridine, cytarabine, 6-mercaptopurine, thioguanine, pentostatin), vinca alkaloids (vincristine, vinblastine, vindesine), epipodophyllotoxins (etoposide, etoposide orthoquinone, and teniposide), antibiotics (daunorubicin, doxorubicin, mitoxantrone, bisanthrene, actinomycin D, plicamycin, puromycin, and gramicidine D), paclitaxel, colchicine, cytochalasin B, emetine, maytansine, and amsacrine. Additional agents include aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethlstilbestrol, docetaxel, dromostanolone, epirubicin, erlotinib, estramustine, etoposide, ethinyl estradiol, exemestane, floxuridine, 5-flourouracil, fludarabine, flutamide, fulvestrant, gefitinib, gemcitabine, goserelin, hydroxyurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, and zoledronate. Other suitable agents are those that are approved for human use, including those that will be approved, as chemotherapeutics or radiotherapeutics, and known in the art. Such agents can be referenced through any of a number of standard physicians' and oncologists' references (e.g., Goodman & Gilman's The Pharmacological Basis of Therapeutics, Ninth Edition, McGraw-Hill, N.Y., 1995) or through the National Cancer Institute website (fda.gov/cder/cancer/druglistfrarne.htm), both as updated from time to time.

Immunomodulatory drugs (IMiDs) such as thalidomide, lenalidomide, and pomalidomide stimulate both NK cells and T cells. As provided herein, IMiDs may be used with the iPSC-derived therapeutic immune cells for cancer treatments.

Other than an isolated population of iPSC-derived hematopoietic lineage cells included in the therapeutic compositions, the compositions suitable for administration to a patient can further include one or more pharmaceutically acceptable carriers (additives) and/or diluents (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition. Accordingly, there is a wide variety of suitable formulations of therapeutic compositions of embodiments of the present invention (see, e.g., Remington's Pharmaceutical Sciences, 17^{th} ed. 1985, the disclosure of which is hereby incorporated by reference in its entirety).

In one embodiment, the therapeutic composition comprises the pluripotent cell derived T cells made by the methods and composition disclosed herein. In one embodiment, the therapeutic composition comprises the pluripotent cell derived NK cells made by the methods and composition disclosed herein. In one embodiment, the therapeutic composition comprises the pluripotent cell derived CD34⁺ HE cells made by the methods and composition disclosed herein. In one embodiment, the therapeutic composition comprises the pluripotent cell derived HSCs made by the methods and composition disclosed herein. In one embodiment, the therapeutic composition comprises the pluripotent cell derived MDSC made by the methods and composition disclosed herein. A therapeutic composition comprising a population of iPSC-derived hematopoietic lineage cells as disclosed herein can be administered separately by intravenous, intraperitoneal, enteral, or tracheal administration methods or in combination with other suitable compounds to affect the desired treatment goals.

These pharmaceutically acceptable carriers and/or diluents can be present in amounts sufficient to maintain a pH of the therapeutic composition of between about 3 and about 10. As such, a buffering agent can be as much as about 5% on a weight to weight basis of the total composition. Electrolytes such as, but not limited to, sodium chloride and potassium chloride can also be included in the therapeutic composition. In one aspect, the pH of the therapeutic composition is in the range from about 4 to about 10. Alternatively, the pH of the therapeutic composition is in the range from about 5 to about 9, from about 6 to about 9, or from about 6.5 to about 8. In another embodiment, the therapeutic composition includes a buffer having a pH in one of said pH ranges. In another embodiment, the therapeutic composition has a pH of about 7. Alternatively, the therapeutic composition has a pH in a range from about 6.8 to about 7.4. In still another embodiment, the therapeutic composition has a pH of about 7.4.

The invention also provides, in part, the use of a pharmaceutically acceptable cell culture medium in particular compositions and/or cultures of embodiments of the present invention. Such compositions are suitable for administration to human subjects. Generally speaking, any medium that supports the maintenance, growth, and/or health of the iPSC-derived effector cells in accordance with embodiments of the invention are suitable for use as a pharmaceutical cell culture medium. In particular embodiments, the pharmaceutically acceptable cell culture medium is a serum free, and/or feeder-free medium. In various embodiments, the serum-free medium is animal-free, and can optionally be protein-free. Optionally, the medium can contain biopharmaceutically acceptable recombinant proteins. Animal-free medium refers to medium wherein the components are derived from non-animal sources. Recombinant proteins replace native animal proteins in animal-free medium and the nutrients are obtained from synthetic, plant or microbial sources. Protein-free medium, in contrast, is defined as substantially free of protein. One having ordinary skill in the art would appreciate that the above examples of media are illustrative and in no way limit the formulation of media suitable for use in the present invention and that there are many suitable media known and available to those in the art.

The iPSC-derived hematopoietic lineage cells can have at least 50%, 60%, 70%, 80%, 90%, 95%, 98%, or 99% T cells, NK cells, NKT cells, proT cells, proNK cells, CD34+HE cells, HSCs, B cells, myeloid-derived suppressor cells (MDSCs), regulatory macrophages, regulatory dendritic cells, or mesenchymal stromal cells. In some embodiments, the iPSC-derived hematopoietic lineage cells have about 95% to about 100% T cells, NK cells, proT cells, proNK cells, CD34⁺ HE cells, or myeloid-derived suppressor cells (MDSCs). In some embodiments, the present invention provides therapeutic compositions having purified T cells or NK cells, such as a composition having an isolated population of about 95% T cells, NK cells, proT cells, proNK cells, CD34⁺ HE cells, or myeloid-derived suppressor cells (MDSCs) to treat a subject in need of the cell therapy.

As a person of ordinary skill in the art would understand, both autologous and allogeneic hematopoietic lineage cells derived from iPSC based on the methods and composition herein can be used in cell therapies as described above. For autologous transplantation, the isolated population of derived hematopoietic lineage cells are either complete or partial HLA-match with the patient. In another embodiment, the derived hematopoietic lineage cells are not HLA-matched to the subject, wherein the derived hematopoietic lineage cells are NK cells or T cells comprising a Fas redirector, wherein the Fas redirector comprises an extracellular Fas binding domain comprising a partial or full peptide of an extracellular domain (ECD) of a Fas receptor (FAS), or a variant or an allele thereof, and a cytoplasmic signaling domain comprising a partial or full peptide of an intracellular domain (ICD) of one or more co-stimulation molecules, wherein the Fas redirector redirects Fas signaling upon binding to a FAS agonist, and wherein the NK cells or T cells optionally comprise one or more modalities listed in Table 1.

In some embodiments, the number of derived hematopoietic lineage cells in the therapeutic composition is at least 0.1×10⁵ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 5×10⁸ cells, at least 1×10⁹ cells, or at least 5×10⁹ cells, per dose. In some embodiments, the number of derived hematopoietic lineage cells in the therapeutic composition is about 0.1×10⁵ cells to about 1×10⁶ cells, per dose; about 0.5×10⁶ cells to about 1×10⁷ cells, per dose; about 0.5×10⁷ cells to about 1×10⁸ cells, per dose; about 0.5×10⁸ cells to about 1×10⁹ cells, per dose; about 1×10⁹ cells to about 5×10⁹ cells, per dose; about 0.5×10⁹ cells to about 8×10⁹ cells, per dose; about 3×10⁹ cells to about 3×10¹⁰ cells, per dose, or any range in-between. Generally, 1×10⁸ cells/dose translates to 1.67×10⁶ cells/kg for a 60 kg patient/subject.

In one embodiment, the number of derived hematopoietic lineage cells in the therapeutic composition is the number of immune cells in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.75×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, at least 30×10⁶ cells/kg of bodyweight, 1×10⁸ cells/kg of bodyweight, 5×10⁸ cells/kg of bodyweight, or 1×10⁹ cells/kg of bodyweight.

In one embodiment, a dose of derived hematopoietic lineage cells is delivered to a subject. In one illustrative embodiment, the effective amount of cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

In some embodiments, the therapeutic use of derived hematopoietic lineage cells is a single-dose treatment. In some embodiments, the therapeutic use of derived hematopoietic lineage cells is a multi-dose treatment. In some embodiments, the multi-dose treatment is one dose every day, every 3 days, every 7 days, every 10 days, every 15 days, every 20 days, every 25 days, every 30 days, every 35 days, every 40 days, every 45 days, or every 50 days, or any number of days in-between.

The compositions comprising a population of derived hematopoietic lineage cells of the invention can be sterile, and can be suitable and ready for administration (i.e., can be administered without any further processing) to human patients/subjects. A cell-based composition that is ready for administration means that the composition does not require any further processing or manipulation prior to transplant or administration to a subject. In other embodiments, the invention provides an isolated population of derived hematopoietic lineage cells that are expanded and/or modulated prior to administration with one or more agents including small chemical molecules. The compositions and methods for modulating immune cells including iPSC-derived effector cells are described in greater detail, for example, in International Pub. No. WO2017/127755, the relevant disclosure of which is incorporated herein by reference. For derived hematopoietic lineage cells that are genetically engineered to express recombinant TCR or CAR, the cells can be activated and expanded using methods as described, for example, in U.S. Pat. No. 6,352,694.

In certain embodiments, the primary stimulatory signal and the co-stimulatory signal for the derived hematopoietic lineage cells can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). Alternatively, one agent can be coupled to a surface and the other agent in solution. In one embodiment, the agent providing the co-stimulatory signal can be bound to a cell surface and the agent providing the primary activation signal is in solution or coupled to a surface. In certain embodiments, both agents can be in solution. In another embodiment, the agents can be in soluble form, and then cross-linked to a surface, such as a cell expressing Fc receptors or an antibody or other binding agent which will bind to the agents such as disclosed in U.S. Pub. Nos. 2004/0101519 and 2006/0034810, the disclosures of which are incorporated by reference, for artificial antigen presenting cells (aAPCs) that are contemplated for use in activating and expanding T lymphocytes in embodiments of the present invention.

Some variation in dosage, frequency, and protocol will necessarily occur depending on the condition of the subject being treated. The person responsible for administration will, in any event, determine the appropriate dose, frequency and protocol for the individual subject.

### EXAMPLES

The following examples are offered by way of illustration and not by way of limitation.

### Example 1—Materials and Methods

An hiPSC platform was used for single cell passaging and high-throughput, 96-well plate-based flow cytometry sorting, to allow for the derivation of clonal hiPSCs with single or multiple genetic modulations.

hiPSCMaintenance in Small Molecule Culture: hiPSCs were routinely passaged as single cells once confluency of the culture reached 75%-90%. For single-cell dissociation, hiPSCs were washed once with PBS (Mediatech) and treated with Accutase (Millipore) for 3-5 min at 37° C. followed with pipetting to ensure single-cell dissociation. The single-cell suspension was then mixed in equal volume with conventional medium, centrifuged at 225×g for 4 min, resuspended in FMM, and plated on Matrigel-coated surface. Passages were typically 1:6-1:8, transferred tissue culture plates previously coated with Matrigel for 2-4 hr in 37° C. and fed every 2-3 days with FMM. Cell cultures were maintained in a humidified incubator set at 37° C. and 5% CO₂.

Human iPSC engineering with ZFN, CRISPR for targeted editing of modalities of interest: Using ROSA26 targeted insertion as an example, for ZFN mediated genome editing, 2 million iPSCs were transfected with mixture of 2.5 μg ZFN-L (FTV893), 2.5 μg ZFN-R (FTV894) and 5 μg donor construct, for AAVS1 targeted insertion. For CRISPR mediated genome editing, 2 million iPSCs were transfected with mixture of 5 μg ROSA26-gRNA/Cas9 (FTV922) and 5 μg donor construct, for ROSA26 targeted insertion. Transfection was done using Neon transfection system (Life Technologies) using parameters 1500V, 10 ms, 3 pulses. On day 2 or 3 after transfection, transfection efficiency was measured using flow cytometry if the plasmids contain artificial promoter-driver GFP and/or RFP expression cassette. On day 4 after transfection, puromycin was added to the medium at concentration of 0.1p g/ml for the first 7 days and 0.2 μg/ml after 7 days to select the targeted cells. During the puromycin selection, the cells were passaged onto fresh matrigel-coated wells on day 10. On day 16 or later of puromycin selection, the surviving cells were analyzed by flow cytometry for GFP⁺ iPS cell percentage.

Bulk sort and clonal sort of genome-edited iPSCs: iPSCs with genomic targeted editing using ZFN or CRISPR-Cas9 were bulk sorted and clonal sorted of GFP⁺SSEA4⁺TRA181⁺ iPSCs after 20 days of puromycin selection. Single cell dissociated targeted iPSC pools were resuspended in chilled staining buffer containing Hanks' Balanced Salt Solution (MediaTech), 4% fetal bovine serum (Invitrogen), 1× penicillin/streptomycin (Mediatech) and 10 mM Hepes (Mediatech); made fresh for optimal performance. Conjugated primary antibodies, including SSEA4-PE, TRA181-Alexa Fluor-647 (BD Biosciences), were added to the cell solution and incubated on ice for 15 minutes. All antibodies were used at 7 μL in 100 μL staining buffer per million cells. The solution was washed once in staining buffer, spun down at 225 g for 4 minutes and resuspended in staining buffer containing 1 μM Thiazovivn and maintained on ice for flow cytometry sorting. Flow cytometry sorting was performed on FACS Aria II (BD Biosciences). For bulk sort, GFP⁺SSEA4⁺TRA181⁺ cells were gated and sorted into 15 ml canonical tubes filled with 7 ml FMM. For clonal sort, the sorted cells were directly ejected into 96-well plates using the 10 μM nozzle, at concentrations of 3 events per well. Each well was prefilled with 200 μL FMM supplemented with 5 μg/mL fibronectin and 1× penicillin/streptomycin (Mediatech) and previously coated overnight with 5× Matrigel. 5× Matrigel precoating includes adding one aliquot of Matrigel into 5 mL of DMEM/F12, then incubated overnight at 4° C. to allow for proper resuspension and finally added to 96-well plates at 50 μL per well followed by overnight incubation at 37° C. The 5× Matrigel is aspirated immediately before the addition of media to each well. Upon completion of the sort, 96-well plates were centrifuged for 1-2 min at 225 g prior to incubation. The plates were left undisturbed for seven days. On the seventh day, 150 μL of medium was removed from each well and replaced with 100 μL FMM. Wells were refed with an additional 100 μL FMM on day 10 post sort. Colony formation was detected as early as day 2 and most colonies were expanded between days 7-10 post sort. In the first passage, wells were washed with PBS and dissociated with 30 μL Accutase for approximately 10 min at 37° C. The need for extended Accutase treatment reflects the compactness of colonies that have sat idle in culture for prolonged duration. After cells are seen to be dissociating, 200 μL of FMM is added to each well and pipetted several times to break up the colony. The dissociated colony is transferred to another well of a 96-well plate previously coated with 5× Matrigel and then centrifuged for 2 min at 225 g prior to incubation. This 1:1 passage is conducted to spread out the early colony prior to expansion. Subsequent passages were done routinely with Accutase treatment for 3-5 min and expansion of 1:4-1:8 upon 75-90% confluency into larger wells previously coated with 1× Matrigel in FMM. Each clonal cell line was analyzed for GFP fluorescence level and TRA1-81 expression level. Clonal lines with near 100% GFP⁺ and TRA1-81⁺ were selected for further PCR screening and analysis. Flow cytometry analysis was performed on Guava EasyCyte 8 HT (Millipore) and analyzed using Flowjo (FlowJo, LLC).

Flow Cytometry of Effectors: For CAR19-iT and primary T cell experiments, cells were stained with a fixable viability dye (BD Biosciences, San Jose, CA) and then washed with PBS. For CD19-CAR detection, the cells were incubated with Alexa Flour 647 conjugated goat anti-mouse IgG F(ab)2 (Jackson ImmunoResearch Laboratories, West Grove PA). Cells were subsequently washed and stained with Pe-Cy7 conjugated anti-CD90.2 (eBioscience) and PE-conjugated anti-TGFβR2 (Biolegend, San Diego, CA). For intracellular staining, cells were fixed with BD Phosflow Fix Buffer (BD Biosciences, San Jose, CA), and permeabilization with BD Phosflow Perm Buffer (BD Biosciences, San Jose, CA). Following permeabilization, samples were incubated with PE-CF594-conjugated anti-STAT5 (pY694) (BD Biosciences, San Jose, CA) and BV421-conjugated anti-pSTAT3 (S727) (BD Biosciences, San Jose, CA). After washing, all samples were resuspended in PBS with 0.2% human serum albumin containing counting beads. All sample data were acquired using LSRFortessa™ X-20 (BD Biosciences, San Jose, CA) and analyzed using Flowjo™ software (Ashland, OR).

For CAR-iNK cells, cells were stained with a fixable viability dye (BD Biosciences, San Jose, CA). After washing, cells were incubated with 10 μg/ml of recombinant human NKG2D blocking antibody (Biolegend, San Diego, CA) and 0.5 mg/ml of recombinant FC Avidin (R&D systems, Minneapolis, MN) on ice. After washing, cells were incubated with PE-conjugated streptavidin followed by biotinylated anti-strepavidin (Vector laboratories, Burlingame, CA). CAR frequency and counts were measured by flow cytometry using counting beads. All sample data were acquired using LSRFortessa™ X-20 (BD Biosciences, San Jose, CA) and analyzed using Flowjo™ software (Ashland, OR).

RNASeq Assay: Total RNA was isolated from 0.1-1×10⁶ cells using the Arcturus™ PicoPure™ RNA Isolation Kit (Applied Biosystems by Thermo Fisher Scientific, Ref 12204-01, Waltham, MA USA) according to the manufacturer's protocol. DNase digestion was performed during RNA purification using the Qiagen RNase-Free DNA Set (Qiagen, Cat No. 79254, Germantown, MD USA). After RNA isolation, the integrity of each sample's RNA was checked using an RNA ScreenTape (Agilent, PN 5067-5576, Santa Clara, CA USA) run on the Agilent 4200 TapeStation System (Agilent, PN G2991AA, Santa Clara, CA USA).

For each sample, 100 ng of total RNA was used to generate RNA sequencing libraries following the manufacturer's protocol using the KAPA mRNA HyperPrep Kit (Roche, Kit KK8581, Wilmington, MA USA). Poly(A) RNA was captured with magnetic oligo-dT beads to produce mRNA capture material which was then fragmented to 200-300 bp in the presence of Mg2+ and incubated for 6 minutes at 94° C. First Strand Synthesis waw done by synthesizing 1st strand cDNA with random primers incubated for 10 minutes at 25° C., 15 minutes at 42° C., and 15 minutes at 70° C. Second Strand Synthesis and A-tailing was performed by incubating samples for 30 minutes at 16° C., and 10 minutes at 62° C. Adaptor Ligation was done by ligating 3′-dTMP adapters to 3′-dAMP library fragments and incubating for 15 minutes at 20° C. Two bead-based Cleanups was done at 0.63× and 0.7× volumes respectively. Production of final libraries for sequencing was performed via PCR amplification of the adapter ligated library incubated for 45 seconds at 98° C.; 15 cycles of 15 seconds at 98° C., 30 seconds at 60° C., 30 seconds at 72° C.; and 1 minute at 72° C. Finally, a 1× volume bead-based library cleanup was performed.

Sequencing libraries were quantified on the Qubit 3 Fluorometer (Invitrogen by Thermo Fisher Scientific, Ref Q33216, Waltham, MA USA) using the Qubit dsDNA HS Assay kit (Invitrogen by Thermo Fisher Scientific, Ref Q32854, Waltham, MA USA). Sequencing library quality and size were assessed on the Agilent 4200 TapeStation System (Agilent, PN G2991AA, Santa Clara, CA USA) using an Agilent High Sensitivity D1000 ScreenTape (Agilent, PN 5067-5584, Santa Clara, CA USA). The libraries were pooled for high-throughput sequencing, and pools were quantified using the KAPA Library Quant Kit-Illumina (Roche, Kit KK4835, Wilmington, MA USA). The quality and size of the pooled libraries was determined on the Agilent 4200 TapeStation System (Agilent, PN G2991AA, Santa Clara, CA USA) using an Agilent High Sensitivity D1000 ScreenTape (Agilent, PN 5067-5584, Santa Clara, CA USA).

Pooled libraries were sequenced on an Illumina NextSeq 500/550 or 2000 platform with 76×76 pair-end reads targeting a minimum of 10 million reads per sample.

RNASeq Analysis: Raw sequencing data from Illumina's NextSeq 500/550 or 2000 platform was coverted to fastq format via the bcl2fastq program (Illumina). Fastq files were cleaned and prepared for alignment using TrimGalore (www.bioinformatics.babraham.ac.uk/projects/trim_galore/). Salmon (Nature Methods, doi.org/10.1038/nmeth.4197) was used to quantify gene expression based on an Hg38 reference genome and transcriptome. The DEseq2 package (Genome Biology doi:10.1186/s13059-014-0550-8) in R (R Core Team—2017) was used to perform differential gene expression analysis and a variance stabilization transformation (VST). Variance-stabilized gene expression data was analyzed via hierarchical clustering and principal component analysis in R using the base package ‘stats’.

### Example 2—Design of Fas Redirection Constructs

While iPSC-derived CAR T (CAR iT) and CAR NK cells (CAR iNK) share many similar features to primary CAR T and CAR NK cells, unique features that are impactful were observed in these iPSC-derived effector cells. Both iPSC-derived T (iT) and primary T cells have high levels of Fas receptor (Fas). Fas is a death receptor that when bound to its respective ligand, Fas ligand (FasL), can induce apoptosis. iPSC-derived T cells begin upregulating Fas during differentiation and have high levels of Fas protein expression at the end of differentiation by both RNA and surface protein expression. This endogenous expression of Fas is highly capable of transmitting apoptotic signals, and thus iT cells are susceptible to apoptosis via Fas ligand (FasL) signaling. iPSC-derived T cells, on the other hand, unlike primary T cells, exhibit elevated levels of FasL RNA expression at baseline and upon activation, suggesting that the FasL expression present on an iT is sufficient to induce fratricide towards other iTs. As compared to primary CAR-T cells, it was further notable that the expression of AP-1 family members such as FOS, FOSB, JUN, JUNB, JUND, critical for inducing AP-1 activity during T cell signaling, are lacking in the iPSC-derived effector cells, which may lead to cell exhaustion. Furthermore, the iPSC-derived effector cells using the method and compositions disclosed herein lack endogenous expression of many co-stimulatory molecules including CD137, CD27, OX40, CD40L, and CD2, which are relevant to the expression of early memory associated transcription factors. It was also observed that the high levels of Fas and FasL exist in iPSC-derived NK cells as well.

Multiple liquid and solid tumors, as well as activated peripheral B, T and NK cells express high levels of FasL. It is hypothesized here that this elevated expression of FasL in a recipient represents a large hurdle to adoptive immune cell products, including CAR T and CAR NK cells. Without being limited by theory, these therapeutic cell products could be Fas positive either by canonical expression or through FasL-induced activation of Fas, thereby being targeted and killed through FasL-induced apoptosis in a tumor environment, or through fratricide by neighboring product cells that express Fas ligand. To test this hypothesis and provide suitable genetic enhancements to adoptive cell products, Fas redirectors were contemplated and designed. To overcome these challenges, genetic enhancements as disclosed herein, including the strategy of diverting FAS signaling, were designed and explored for their capability to enhance effector cells, including those derived from iPSCs.

A number of constructs were designed to deliver to a cell an exogenous polynucleotide encoding a signaling redirector receptor that comprises a partial or full peptide of a Fas binding domain (SEQ ID NO: 1; labeled as FAS in various constructs as disclosed) from a Fas receptor, or a variant or an allele thereof, and a cytoplasmic signaling domain comprising a partial or full peptide of an intracellular domain (ICD) of one or more co-stimulation molecules including, but not limited to, CD27, CD28, CD40, MyD88, OX40, IL12Rb2, IL18R1, IL21R, and combinations thereof (the source of respective cytoplasmic signaling domain indicated in “( )” of a construct. Several exemplary Fas redirectors such as FAS-(MyD88-CD40), FAS-(CD27), FAS-(CD28), FAS-(OX40), FAS-(IL12Rβ2), FAS-(IL18R1), and FAS-(IL21R) were constructed (see SEQ ID NOs: 2-8). For analytical purposes, the Fas redirector constructs also contained a 2A cleavage peptide followed by Thy1.2, a small glycoprotein that can serve as a transduction control and selection marker. The transmembrane region of a Fas redirector could be a full or a portion of a transmembrane protein, including a Fas receptor protein and its variant or allele thereof.

In addition, a FAS-(41BB) construct and a dominant negative Fas receptor (dnFAS) construct were prepared for control purposes in various assays.

### Example 3—iPSC-Derived Effector Cells Transduced with Fas Redirection Constructs

To test if Fas redirection constructs affect effector cell expansion and maturation, lentiviral transduction was performed on iPSC-derived CD19-CAR T cells during an intermediate step of the T cell differentiation process followed by a Thy1.2 magnetic bead selection 4 days after transduction.

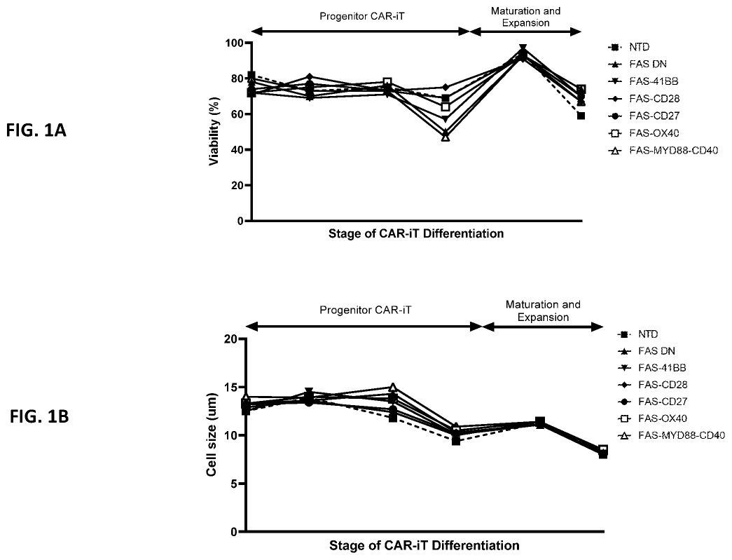

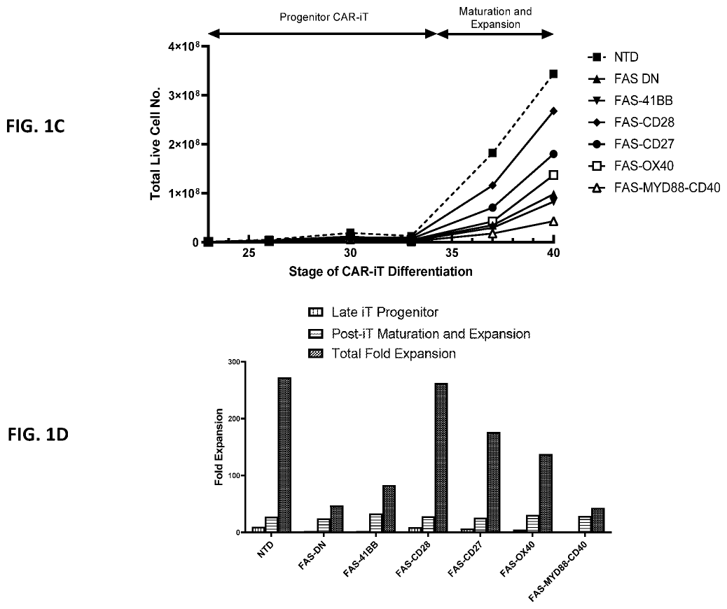

As shown in FIG. 1A, the percentage of live transduced iPSC-derived CD19-CAR T cells throughout T cell differentiation for each Fas construct approximated that of cells transduced with the dominant negative Fas receptor (dnFAS, or FAS DN or FAS DNR) and non-transduced (NTD) cells. While small delays in growth kinetics due to lentiviral transduction/selection were observed, no gross phenotypical changes to critical T cell identity markers, including cell size of live transduced CAR iT cells throughout T cell differentiation, occurred (FIG. 1). As shown in FIG. 1C, the total number of live cells was consistent among all cell groups until about day 33 of T cell differentiation, with FAS-(MyD88-CD40) transduced cell group having the least live cell count in comparison to other constructs. FIG. 1D summarizes the fold expansion of transduced iPSC-derived CAR iT cells during late iT progenitor, post-iT maturation and expansion, and total fold expansion.

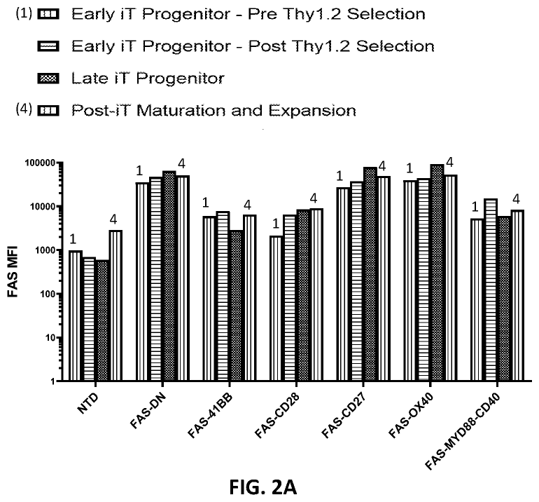

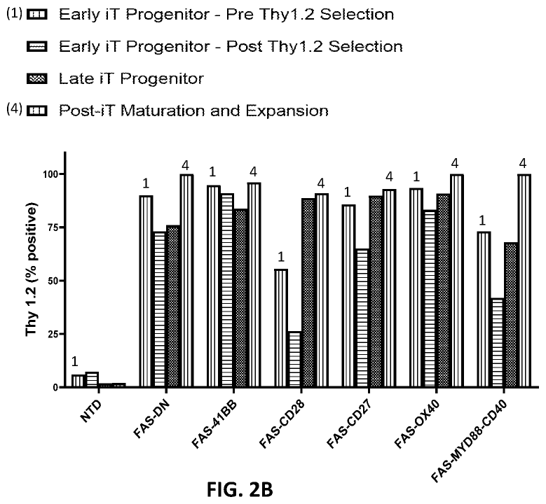

Additionally, higher levels of various indicated FAS construct expression, quantified by FAS mean fluorescence intensity (MFI), were observed in the transduced populations compared to non-transduced (NTD) cells (FIG. 2A). Moreover, the percent positive of Thy1.2 on transduced iPSC-derived CAR iT cells throughout T cell differentiation was quantified. The high levels of Thy1.2 marker (91%+) were observed in transduced cell groups, indicating high levels of transduction (FIG. 2B).

Collectively, the data shows that the transduced FAS redirection constructs as designed support iPSC-derived effector cell expansion in a scalable manner.

### Example 4—FAS Redirection Improves Effector Cell Functionality

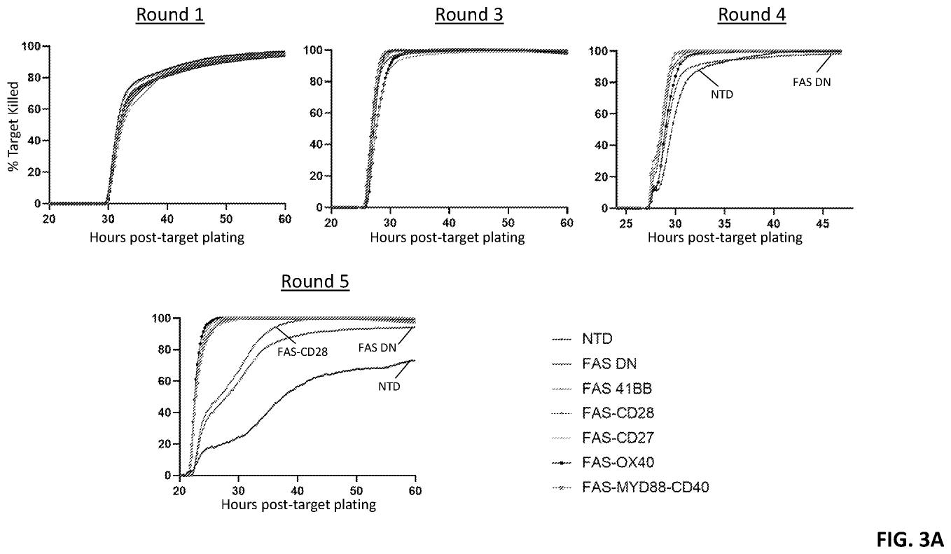

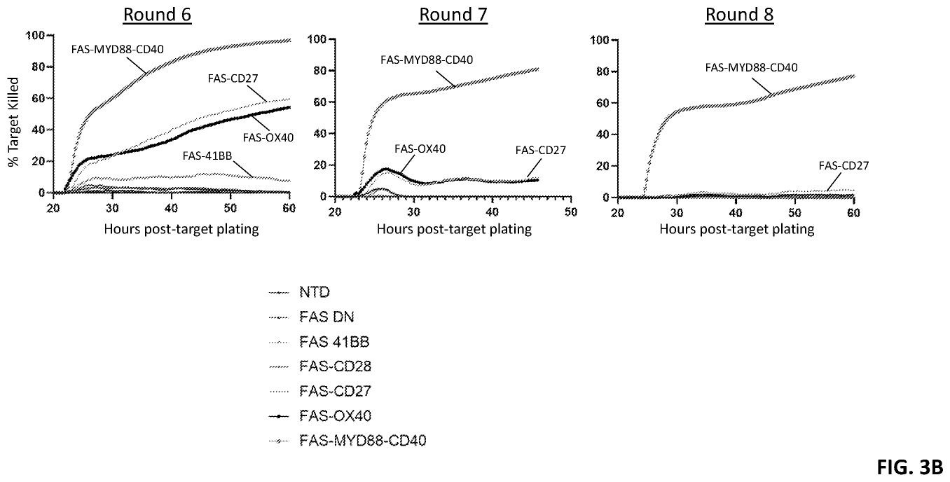

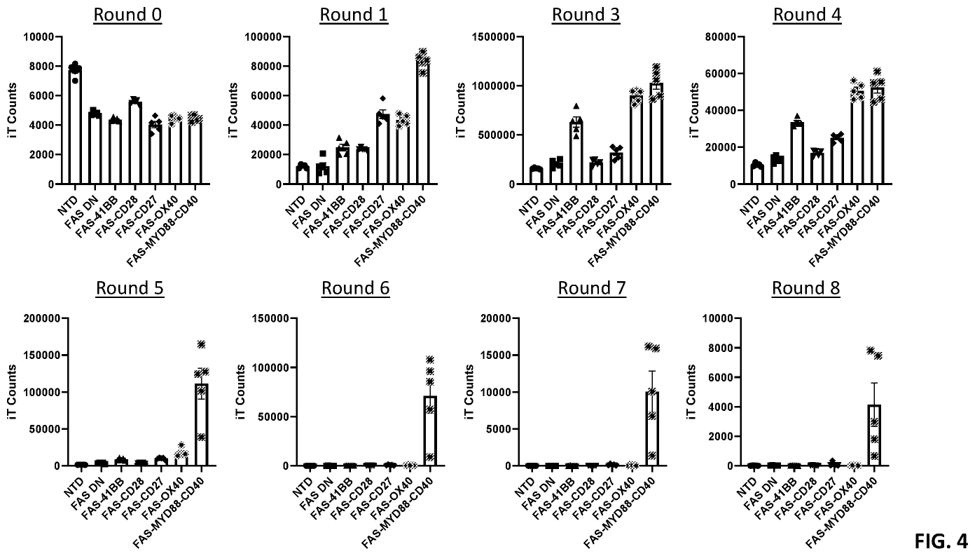

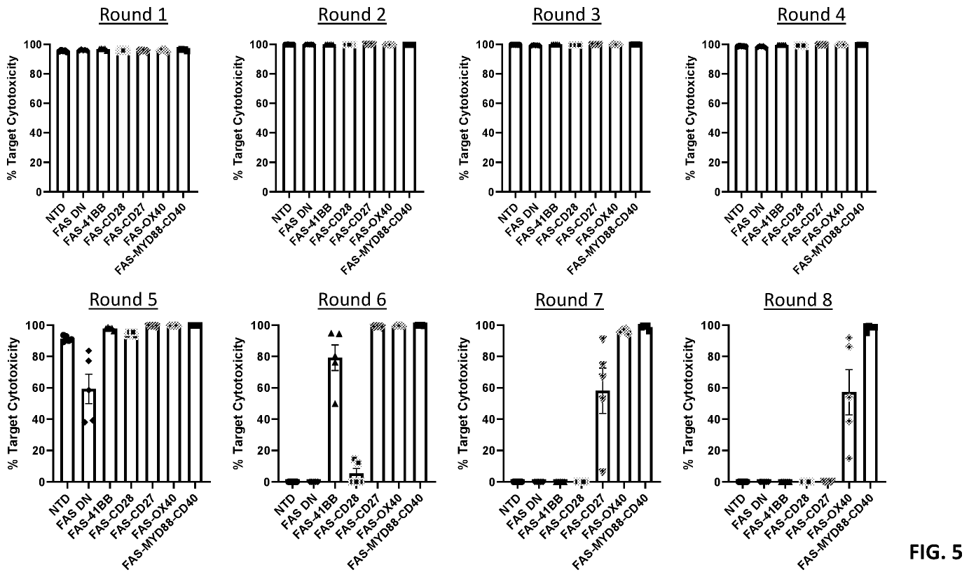

To test whether FAS redirection could enhance CAR-iT cell anti-tumor function, fully differentiated CAR19-iT cells transduced with redirection constructs, FAS DN, or NTD cells were co-cultured with CD19 overexpressing SKOV3, a solid tumor ovarian cell line, in a serial stimulation assay. In this assay, the CAR-iT cells were restimulated for multiple rounds every 3-4 days with fresh SKOV3 targets cells. As shown in FIGS. 3A and 3B, all groups of CAR-iT cells maintained robust killing during the first four rounds of restimulation. However, those cells transduced with FAS redirection constructs outperformed the NTD or FAS DN expressing CAR iT from and beyond round 5 of restimulation, until the end of the study. The impact to cell expansion also further differentiates among the FAS redirector constructs at about round 5, where the cells expressing FAS redirector that provides MYD88-CD40 intracellular signaling exhibited extraordinary expansion (FIG. 4), which came as a surprise considering that this construct is associated with the inferior live effector cell number after cell differentiation in comparison to other constructs (see FIG. 1C). It was also observed that the expansion and persistence/durability benefits by the FAS redirection constructs also provided prolonged killing of SKOV3 targets; and that the FAS-MYD88-CD40 demonstrated the greatest expansion, persistence, and killing, with FAS-CD27 and FAS-OX40 also outperforming the NTD or FAS DN expressing CAR iT.

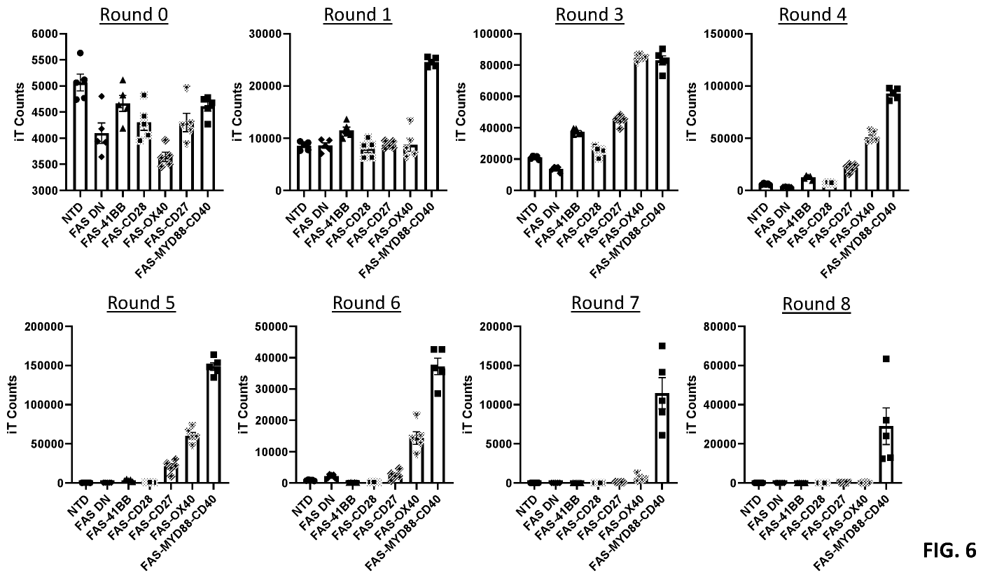

In a separate serial stimulation assay, the same groups of CD19-CAR iT cells were co-cultured with Nalm6, a B cell leukemia line that naturally expresses CD19. The CAR-iT cells were restimulated for multiple rounds every 3-4 days with fresh Nalm6 target cells. All groups of CAR-iT cells maintained robust CD19 directed killing during the first four rounds of restimulation, with cells transduced with FAS redirection constructs outperforming the NTD or FAS DN CAR iT from and beyond round 5 of restimulation till the end of the study (FIG. 5). The differential CAR-directed target cell killing among cell groups was notable starting from and beyond round 6, with FAS-CD27, FAS-OX40 and FAS-MyD88-CD40 supporting effector cytotoxicity better than FAS-41BB (FIG. 6). Between FAS-MyD88-CD40 and FAS-OX40 at round 8, the former again outperformed (FIG. 6).

On a further observation, the FAS-redirected iTs still require exogenous cytokine support, when lacking an expressed cytokine or cytokine signaling complex through genetic engineering of the modality, as disclosed herein. Without IL2 or IL15, these FAS-redirected iTs die within a few rounds of stimulation, which could be potentially utilized as a safety control against unwanted effector cell proliferation.

In view of both restimulation assays, the FAS redirectors, particularly constructs that additionally provide CD27, OX40, or MYD88-CD40 intracellular signaling, present superior effector cell enhancement, including effector cell expansion, persistence, and both innate and CAR intermediated cytotoxicity, indictive of protection of the effector cells from any source of FAS ligand, whether self-made or target-made.

### Example 5—FAS Redirection Constructs Protect Against FAS Agonist-Induced Apoptosis and Activate Intracellular Signaling

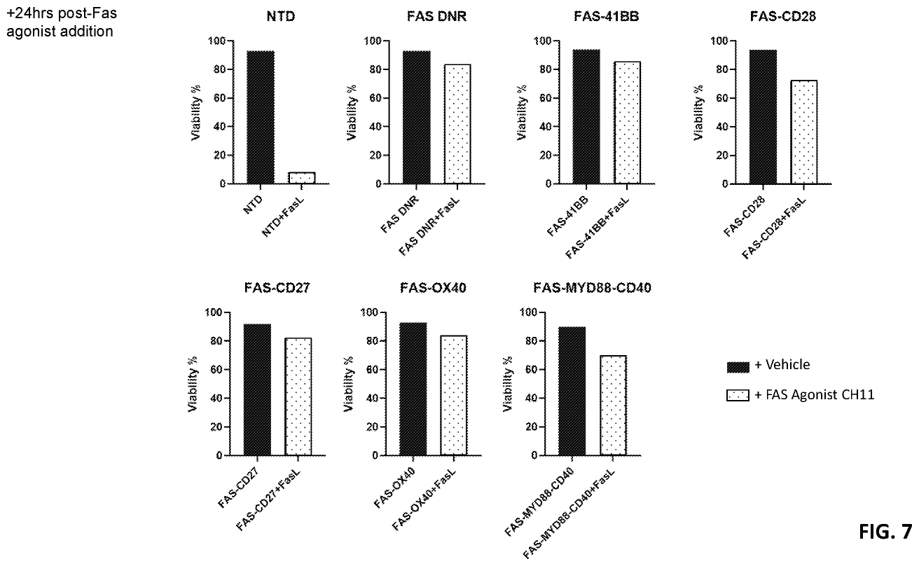

Like primary T cells, iPSC-derived T (iT) cells have high levels of Fas receptor and are therefore susceptible to apoptosis via FasL signaling. Unlike primary T cells, iT cells obtained using the differentiation method and composition as described exhibit higher levels of FasL RNA expression at baseline and upon activation, in comparison, such that the iT cells are also subject to fratricide via FasL signaling among the iT cells. To determine whether the Fas redirector constructs provide protection to iT cells against Fas agonist-induced cell death, the Fas redirector constructs were transduced into an NF-κB luciferase reporter recombinant Jurkat cell line (BPS Bioscience), which were then exposed to a Fas agonist, CH11 (Sigma-Aldrich). After 24 hours, the percentage of viable cells was determined following exposure. As shown in FIG. 7, each of the groups of transduced cells, including the Fas DNR cells, maintained over 60% viability following stimulation with CH11, whereas the NTD cells were not protected from FasL induced apoptosis.

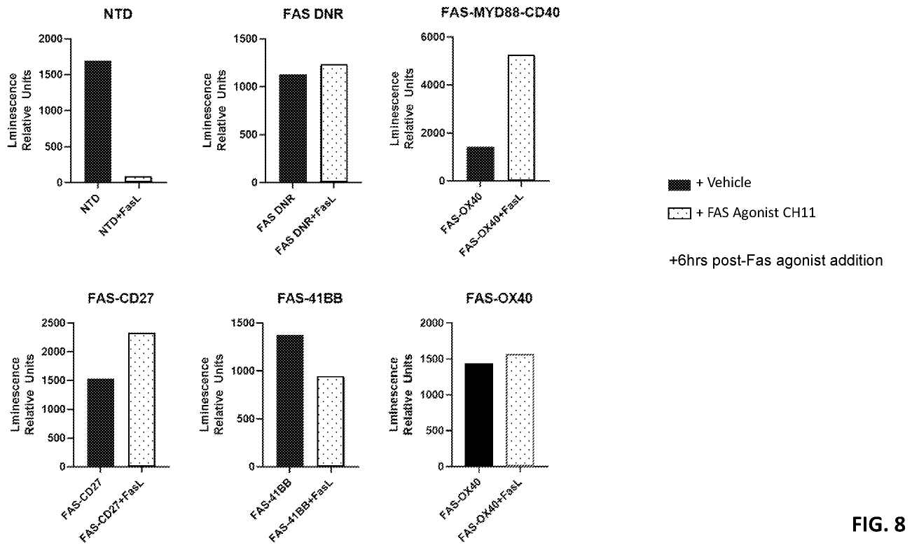

To investigate whether co-stimulation of the intracellular domain of the FAS redirectors produces a signal, the above transduced NF-κB luciferase reporter recombinant Jurkat cells were again stimulated with CH11 at 1 μg/ml. In this assay, cells that receive the external Fas signal and redirect the signal to the intracellular co-stimulation domains are expected to activate the NF-κB reporter line and produce high levels of luciferase. After 6 hours of exposure to the Fas agonist, luminescence relative units, representing activated NF-κB reporter line cells, was calculated. As shown in FIG. 8, the constructs that provide MYD88-CD40 and CD27 intracellular signaling outperformed the FAS DNR and FAS-41BB cells. It is further noted that even though the FAS-41BB transduced cells were provided protection against complete apoptosis, the reduced luminescence relative units as compared to without Fas agonist stimulation is indicative unsuccessful FasL signal redirection through 41BB intracellular signaling domain.

Collectively, the data supports that diverting FAS signaling using the redirector constructs as disclosed is capable of enhancing effector cell proliferation and durability, and preventing exhaustion and apoptosis of these cells, including CAR-iT and CAR-iNK, leading to more efficacious cell products with desirable attributes including, but not limited to, durability and self-protection (against apoptosis, fratricide and the host immune system).

In a further experiment, generation of luciferase by cells transduced with each of the constructs are quantified at 1 hr, 8 hrs, and overnight after Fas agonist addition, and compared to NTD, FAS DNR and FAS-41BB transduced Jurkat cells.

### Example 6—Cytokine Profiling of iT Cells Transduced with Fas Redirection Constructs

CD19-CAR iT cells that have been transduced with the Fas redirection constructs are stimulated against NALM6 targets overnight in the presence of IL-2. Supernatant is frozen after the overnight stimulation and subsequently tested for cytokine secretion. IL-2, perforin, TNFα, and IFNγ cytokine secretion are quantified using a commercially available Ella cartridge (Protein Simple).

In a further experiment, CD19-CAR iT cells are transduced with the Fas redirection constructs during the early iT progenitor phase. Functionality of these cells are compared to CD19-CAR iT cells derived from iPSC engineered to express the Fas redirection constructs in the CD38 locus. In particular, these iT cells are functionally tested in serial stimulation co-cultures with NALM6 and SKOV3 targets engineered to express CD19, as described above. Cytotoxicity of target cells and iT proliferation and persistence under serial stimulation are quantified to evaluate phenotypic and functional impact to Fas redirector and the cells that are specific to insertion time and site.

### Example 7—In Vivo Study of iT Cells Transduced with Fas Redirector

To test the benefits of Fas redirection in vivo, female NSG mice are implanted with 1e5 NALM6.LUC cells intravenously (i.v.). Three days following implantation, 1e6 or 2e6 non-transduced CD19-CAR iT or CD19-CAR iT transduced with respective Fas redirection construct are injected i.v. to the mice alongside IL-2 and IL-15 cytokine support. Tumor growth in each test group is monitored weekly by quantifying luciferase through IVIS Spectrum in vivo imaging. Blood is also be collected weekly to quantify iT counts present in the blood and for phenotypical analysis.

The data presented herein demonstrate that Fas redirector constructs activating selective signaling pathways, while eliminating Fas signaling, are useful to enhance the anti-tumor function of effector cells, including both iT and iNK cells, through enhanced effector cell expansion, persistence, and cytotoxicity while reducing apoptosis, fratricide and preventing exhaustion.

### Example 8—FAS Redirector and Resultant Gene Expression Signature in iT Cells

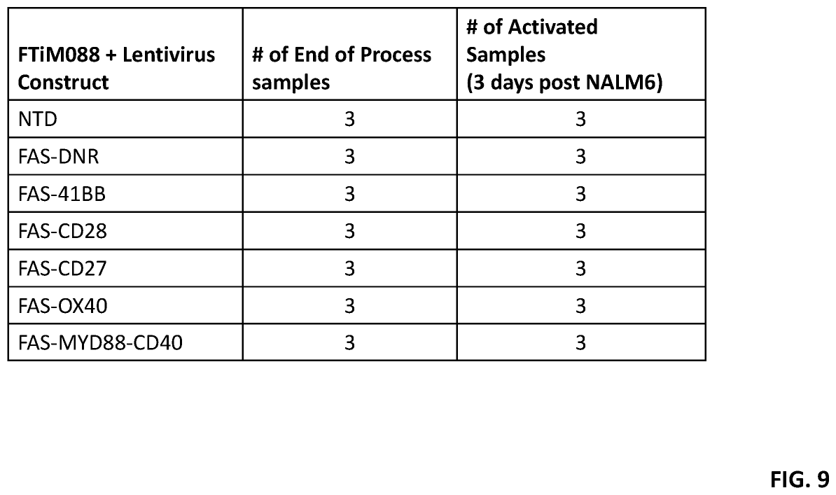

Progenitor CD19 CAR-iT cells were lentivirally transduced with the indicated Fas redirection constructs. Triplicate samples were prepared and collected for end-of-process cells and at 48 hours after activation with NALM6 tumor cells. Total RNA was isolated from the samples and bulk RNAseq was performed. A table providing a list of samples for bulk RNAseq analysis is provided in FIG. 9.

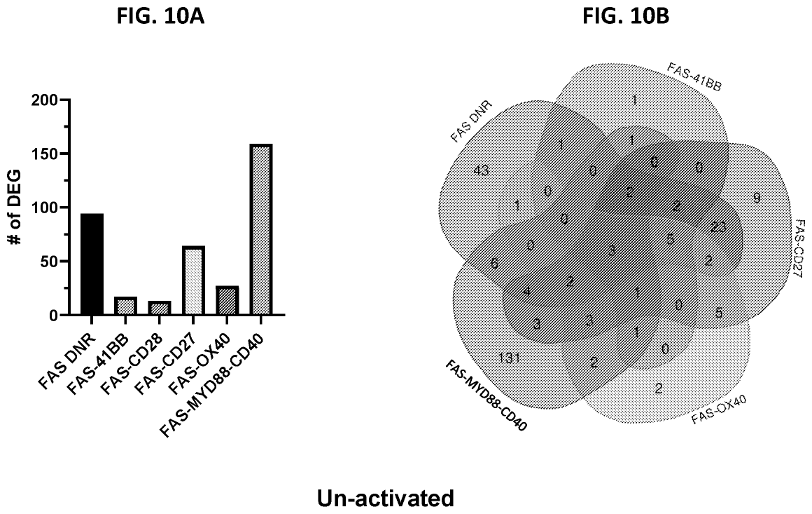

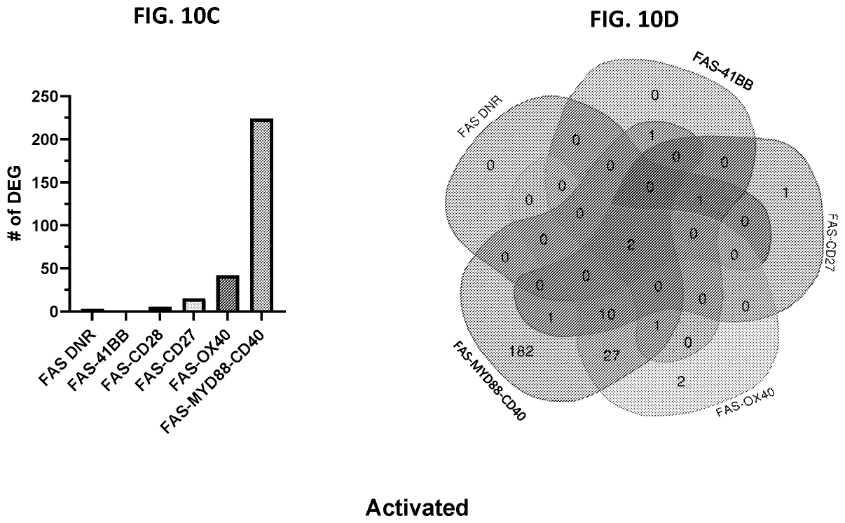

Following bulk RNA sequencing, differentially expressed gene (DEG) analysis was performed on the un-activated (end of process) samples to determine the number of DEGs between the untransduced control compared to each sample transduced with the various Fas redirection constructs. A bar graph of illustrative results for the DEG analysis of un-activated samples is provided in FIG. 10A.

Venn diagram analysis of the DEG list from FIG. 10A is illustrated in FIG. 10B, showing the number of overlapping or uniquely differentially expressed genes in un-activated (end of process) CD19 CAR-iT cells expressing the different Fas redirection constructs.

The same analysis as in FIG. 10A was performed on the activated samples from a 48-hour co-culture with target cells. The CD19 CAR-iT cells expressing Fas-MyD88/CD40 showed the highest number of DEGs compared to untransduced CD19 CAR-iT controls (see FIG. 10C).

The same analysis as in FIG. 10B was performed, but with the activated samples. The venn diagram of FIG. 10D shows that CD19 CAR-iT cells expressing Fas-Myd88/CD40 had 182 differentially expressed genes relative to the untransduced control that are not found in the DEG lists for the untransduced control versus the other Fas redirectors.

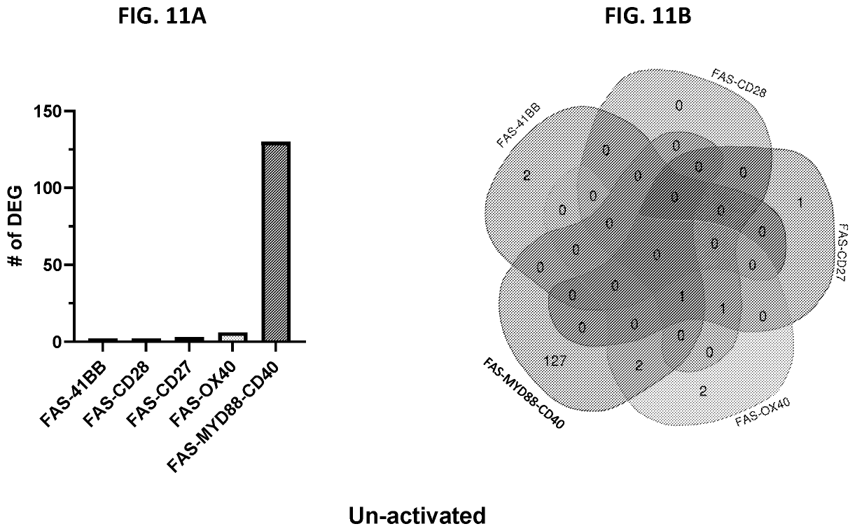

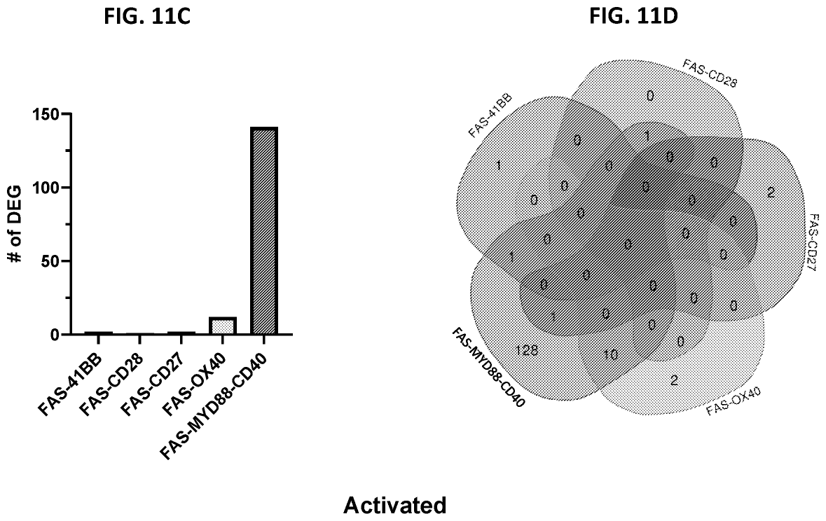

Since Fas signal redirection constructs were designed to not only block Fas signaling but also initiate signaling that is dependent on the associated endodomain, we performed DEG analysis between CAR-iT cells expressing Fas DNR versus the different Fas redirector constructs. Fas-MyD88/CD40 showed the highest number of DEGs compared to Fas DNR in both un-activated (FIG. 11A) and activated (FIG. 11C) conditions. The majority of these DEGs in the Fas-MyD88/CD40 samples were unique, as demonstrated by the venn diagram analysis for un-activated (FIG. 111B) and activated (FIG. 11D) conditions. Pathway analysis indicated most differentially expressed genes in FAS-MyD88/CD40 were controlled by NF-κB and canonical signals downstream of MyD88 or CD40. These results are consistent with those obtained for the NF-κB reporter Jurkat line, discussed in the examples above.

### Example 9—Cells Co-Expressing Fas Redirector with a Recombinant Cytokine Signaling Complex have Enhanced Killing and Persistence

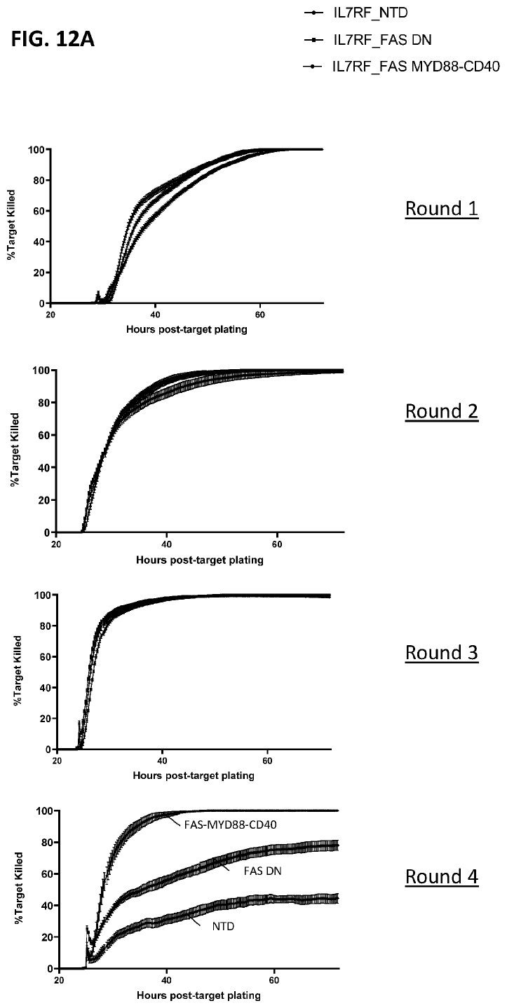

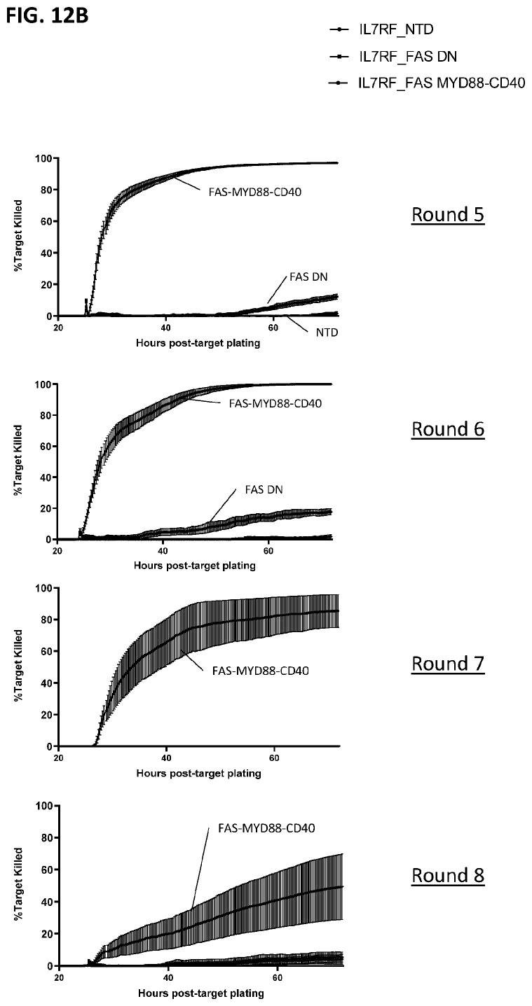

FIGS. 12A-12B provide representative plots of the cytotoxicity of transduced iPSC-derived CD19-CAR T_IL7RF cells against SKOV3 targets that are engineered to express CD19. CD19-CAR iT cells were restimulated for multiple rounds every 3-4 days with fresh SKOV3 targets. Error bars reflect the standard error of the mean (N=5).

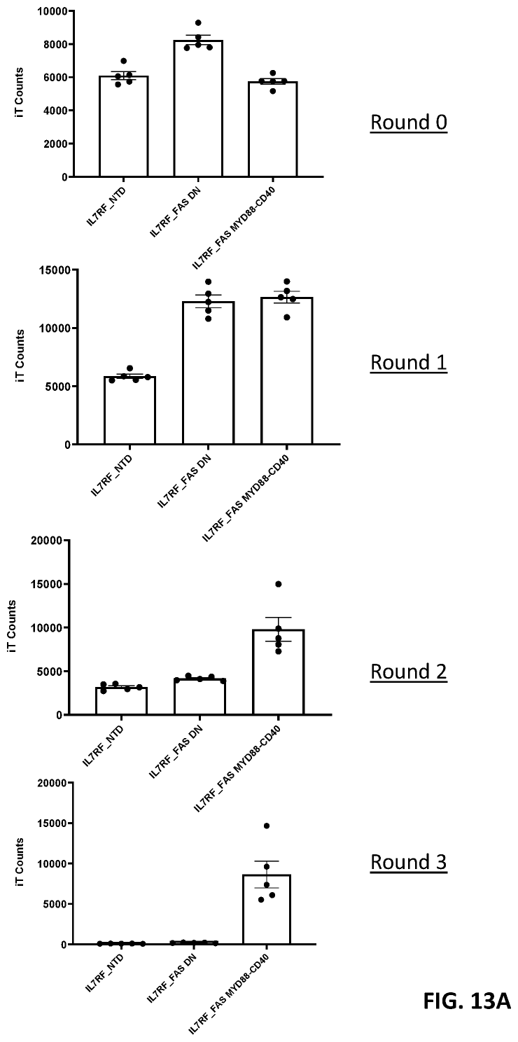

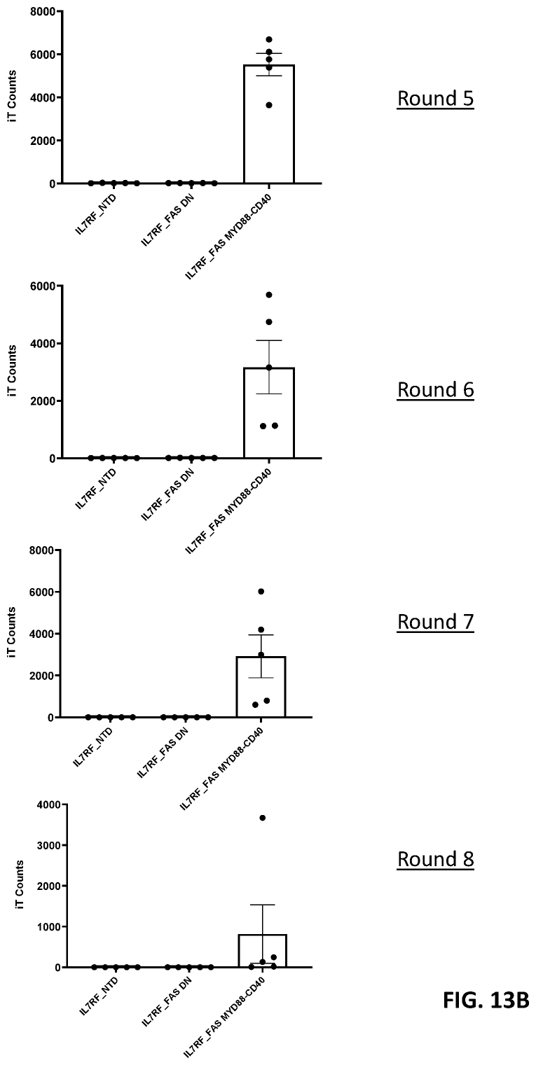

FIGS. 13A-13B provide representative plots of the iT cell counts of transduced iPSC-derived CD19-CAR T_IL7RF cells against SKOV3 targets that are engineered to express CD19. CD19-CAR iT cells were restimulated for multiple rounds every 3-4 days with fresh SKOV3 targets. Error bars reflect the standard error of the mean (N=5). As shown, MyD88/CD40 expressing CD19-CAR_IL7RF iT exhibits enhanced target killing and persistence against engineered SKOV3 that express CD19.

These results demonstrated that Fas-MyD88/CD40 is compatible with CD19-CAR iPSC-derived T cells independent of whether target cells (e.g., NALM6, SKOV3.CD19) actually express Fas ligand. This data, alongside the demonstration that iPSC-derived T cells express Fas ligand, suggests that iT-iT interactions are driving Fas ligand-dependent fratricide. Without wishing to be bound by theory, we hypothesized that this fratricide could present an even greater challenge in iT cells that have enhanced durability or proliferation, and that Fas-MyD88/CD40 may be synergistic in this setting. We observed greater long-term killing capacity and persistence by CD19-CAR iT that express IL7 receptor fusion (IL7RF) and Fas-MyD88/CD40 against SKOV3.CD19 (FIGS. 12A, 12B, 13A, and 13B). The results also indicated that IL7 receptor fusion iT cells could be a good model for testing Fas dependent killing and cell persistence.

### Example 10—Fas-MYD88/CD40 Enhances Tumor Clearance and Prolonged T Cell Activation in the Tumor Microenvironment

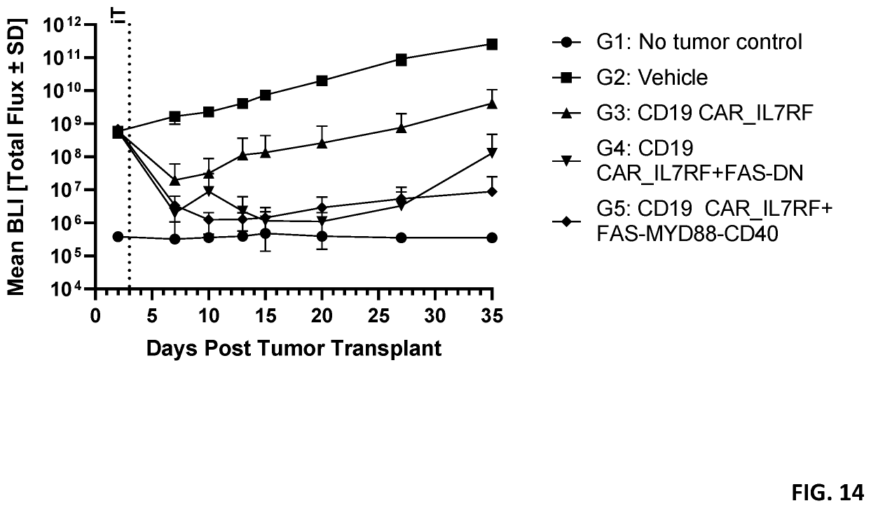

NSG mice were administered with 105 SKOV3.CD19 that overexpress firefly luciferase by i.p injection, engrafted for 3 days, and then administered 10⁷ CAR T cells by i.p. injection (n=8 mice per cohort). IL2 and IL15 was supplied twice a week, and tumor growth was measured weekly by bioluminescence. In the solid tumor ovarian tumor model, SKOV3.CD19, we observed greater tumor clearance by CD19-CAR_IL7RF that are capable of inhibiting Fas signaling compared to CD19-CAR_IL7RF alone. Although not a dramatic difference in tumor clearance between CD19-CAR_IL7RF with Fas dominant negative receptor and Fas-MyD88/CD40 in this model, the latter shows better control of the tumor. Illustrative results showing tumor clearance in this model are provided in FIG. 14.

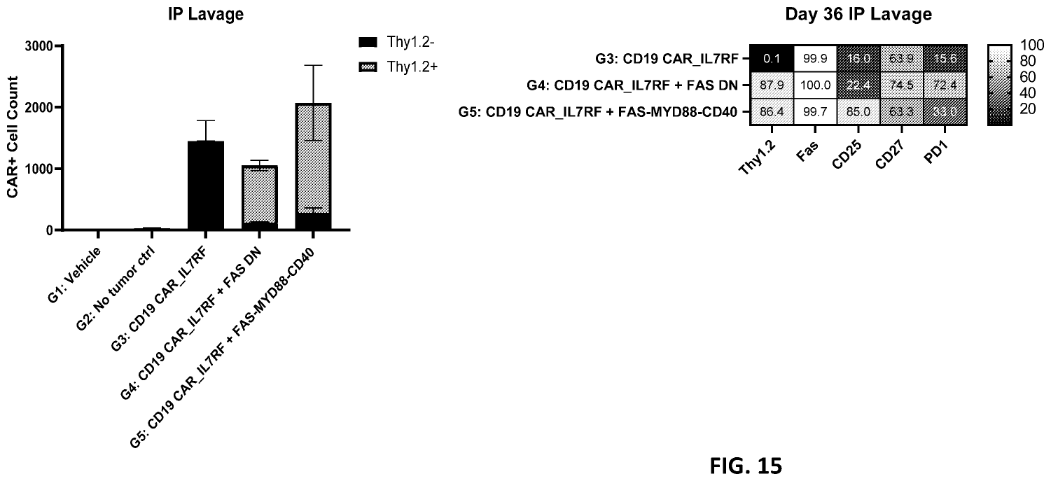

On day 36, 3 mice were lavaged with HBSS, and CAR+ cell count, FAS redirector (Thy1.2+), and activation phenotype was quantified by flow cytometry. Interestingly, we observed that CD19-CAR_IL7RF with Fas-MyD88/CD40 have higher CAR-T counts than other effector groups in the tumor microenvironment. Furthermore, when we phenotyped these cells, we observed a much greater percentage of CD25+ T cells in the Fas-MyD88/CD40 effector group compared to the FAS DN group (85% to 22%) (FIG. 15). This data suggests that FAS redirection to MyD88/CD40 may augment T cell activation in the tumor microenvironment.

The data presented herein demonstrate that Fas redirector constructs activating selective signaling pathways, while eliminating Fas signaling, are useful to enhance the anti-tumor function of effector cells, including both iT and iNK cells, through enhanced effector cell expansion, persistence, and cytotoxicity while reducing apoptosis, fratricide and preventing exhaustion.

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A cell or a population thereof, wherein:
(i) the cell is (a) an immune cell; (b) an induced pluripotent cell (iPSC), a clonal iPSC, or an iPS cell line cell; or (c) a derivative cell obtained from differentiating the cell of (b);
(ii) the cell comprises an exogenous polynucleotide encoding a signaling redirector receptor that comprises:
(a) an extracellular Fas binding domain comprising a partial or full peptide of an extracellular domain (ECD) of a Fas receptor (FAS), or a variant or an allele thereof; and
(b) a cytoplasmic signaling domain comprising a partial or full peptide of an intracellular domain (ICD) of one or more co-stimulation molecules,
wherein the signaling redirector receptor is a Fas redirector that redirects Fas signaling upon binding to a FAS agonist, thereby providing the cell or the derivative cell with improved apoptosis resistance and/or exhaustion resistance.

2. The cell or population thereof of claim 1, wherein
(i) the Fas redirector further comprises a transmembrane region comprising a transmembrane domain, or a portion thereof, of a transmembrane protein;
(ii) the one or more co-stimulation molecules comprise CD27, CD28, CD40, MyD88, OX40, IL12Rβ2, IL18R1, IL21R, or a combination thereof,
(iii) the one or more co-stimulation molecules do not comprise 41BB;
(iv) the FAS agonist comprises a Fas ligand (FasL); or
(v) the cell further comprises one or more of:
(a) an exogenous polynucleotide encoding a chimeric antigen receptor (CAR);
(b) an exogenous polynucleotide encoding a CD16 or a variant thereof,
(c) CD38 knockout; and
(d) an exogenous polynucleotide encoding a cytokine signaling complex comprising a partial or full peptide of a cell surface expressed exogenous cytokine and/or a receptor thereof.

3. The cell or population thereof of claim 2, wherein the transmembrane region of the Fas redirector comprises:
(i) a full length or at least a portion of the native or modified transmembrane region of FAS, CD2, CD3δ, CD3ε, CD3γ, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide; or
(ii) a full or partial length of the transmembrane domain of FAS.

4. The cell or population thereof of claim 1, wherein:
(i) the extracellular binding domain of the Fas redirector comprises a sequence having at least about 85%, about 90%, about 95%, about 96%, about 97%, about 98%, or about 99% identity to SEQ ID NO: 1;
(ii) the cytoplasmic signaling domain of the Fas redirector comprises:
(a) a full or partial length of the intracellular domain (ICD) of MyD88 and CD40, represented by SEQ ID NO: 2; or
(b) a full or partial length of the intracellular domain (ICD) of CD27, represented by SEQ ID NO: 3; or
(c) a full or partial length of the intracellular domain (ICD) of CD28, represented by SEQ ID NO: 4; or
(d) a full or partial length of the intracellular domain (ICD) of OX40, represented by SEQ ID NO: 5; or
(e) a full or partial length of the intracellular domain (ICD) of IL12Rβ2, represented by SEQ ID NO: 6; or
(f) a full or partial length of the intracellular domain (ICD) of IL18R1, represented by SEQ ID NO: 7; or
(g) a full or partial length of the intracellular domain (ICD) of IL21R, represented by SEQ ID NO: 8; or
(iii) the Fas redirector comprises an amino acid sequence that has at least about 85%, about 90%, about 95%, about 96%, about 97%, about 98%, or about 99% identity to any one of SEQ ID NOs: 9-15.

5. The cell or population thereof of any one of claims 1-4, wherein the cell further comprises one or more of:
(i) at least one of the genotypes listed in Table 1;
(ii) HLA-I deficiency and/or HLA-II deficiency;
(iii) introduction of HLA-G or non-cleavable HLA-G, or knockout of one or both of CD58 and CD54;
(iv) deletion or disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or
(v) introduction of at least one of HLA-E, 4-1BBL, CD3, CD4, CD8, CD47, CD113, CD131, CD137, CD80, PDL1, A_{2A}R, antigen-specific TCR, chimeric fusion receptor (CFR), Fc receptor, an antibody or functional variant or fragment thereof, a checkpoint inhibitor, an engager, and surface triggering receptor for coupling with bi- or multi-specific or universal engagers.

6. The cell or population thereof of any one of claims 1-5, wherein the cell comprises HLA-I deficiency and/or HLA-II deficiency; and optionally,
wherein the cell comprises an exogenous polynucleotide encoding HLA-G, HLA-E, or a variant thereof, and/or comprises deletion or disruption of one or both of CD54 and CD58.

7. The cell or population thereof of claim 6, wherein the HLA-I deficiency comprises deletion or disruption of at least one of: B2M, TAP1, TAP2, and Tapasin; or
wherein the HLA-II deficiency comprises deletion or disruption of at least one of: CIITA, RFX5, RFXAP, and RFXANK.

8. The cell or population thereof of any one of claims 1-7, wherein the derivative cell:
(a) comprises a derivative CD34⁺ cell, a derivative hematopoietic stem and progenitor cell, a derivative hematopoietic multipotent progenitor cell, a derivative T cell progenitor, a derivative NK cell progenitor, a derivative T cell, a derivative NKT cell, a derivative NK cell, a derivative B cell, or a derivative effector cell having one or more functional features that are not present in a counterpart primary T, NK, NKT, and/or B cell; or
(b) is an allogeneic effector cell, wherein the effector cell is a derivative NK cell or a derivative T cell having at least one of the following characteristics comprising:
(i) improved persistency and/or survival;
(ii) increased resistance to activated recipient immune cells;
(iii) increased cytotoxicity;
(iv) improved tumor penetration;
(v) enhanced or acquired ADCC;
(vi) enhanced ability in migrating, and/or activating or recruiting bystander immune cells, to tumor sites;
(vii) enhanced ability to reduce tumor immunosuppression;
(viii) improved ability in rescuing tumor antigen escape; and
(ix) reduced apoptosis and/or fratricide,
in comparison to its native counterpart cell obtained from peripheral blood, umbilical cord blood, or other donor tissues.

9. The cell or population thereof of claim 2, wherein the exogenous CD16 comprises at least one of:
(a) a high affinity non-cleavable CD16 (hnCD16) or a variant thereof;
(b) F176V and S197P in ectodomain domain of CD16;
(c) a full or partial ectodomain originated from CD64;
(d) a non-native (or non-CD16) transmembrane domain;
(e) a non-native (or non-CD16) intracellular domain;
(f) a non-native (or non-CD16) signaling domain;
(g) a non-native stimulatory domain; and
(h) transmembrane, signaling, and stimulatory domains that are not originated from CD16, and are originated from a same or different polypeptide.

10. The cell or population thereof of claim 9, wherein:
(a) the non-native transmembrane domain is derived from a CD3δ, CD3ε, CD3γ, CD3ζ, CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor (TCR) polypeptide;
(b) the non-native stimulatory domain is derived from a CD27, CD28, 4-1BB, OX40, ICOS, PD-1, LAG-3, 2B4, BTLA, DAP10, DAP12, CTLA-4, or NKG2D polypeptide;
(c) the non-native signaling domain is derived from a CD3ζ, 2B4, DAP10, DAP12, DNAM1, CD137 (4-1BB), IL21, IL7, IL12, IL15, NKp30, NKp44, NKp46, NKG2C, or NKG2D polypeptide; or
(d) the non-native transmembrane domain is derived from NKG2D, the non-native stimulatory domain is derived from 2B4, and the non-native signaling domain is derived from CD3ζ.

11. The cell or population thereof of claim 1, wherein the CAR is:
(i) T cell specific or NK cell specific;
(ii) a bi-specific antigen binding CAR;
(iii) a switchable CAR;
(iv) a dimerized CAR;
(v) a split CAR;
(vi) a multi-chain CAR;
(vii) an inducible CAR;
(viii) co-expressed with a cytokine signaling complex comprising a partial or full peptide of a cell surface expressed exogenous cytokine and/or a receptor thereof, optionally in separate constructs or in a bi-cistronic construct;
(ix) co-expressed with a checkpoint inhibitor, optionally in separate constructs or in a bi-cistronic construct; and/or
(x) optionally inserted at:
(1) a TRAC or a TRBC locus, and/or is driven by an endogenous promoter of TCR, and/or the TCR is knocked out by the CAR insertion;
(2) a safe harbor locus; or
(3) a gene locus intended for disruption.

12. The cell or population thereof of claim 1, wherein the CAR is:
(i) specific to at least one of CD19, BCMA, B7H3, CD20, CD22, CD38, CD52, CD79b, CD123, EGFR, EGP2/EpCAM, GD2, GPRC5D, HER2, KLK2, MICA/B, MR1, MSLN, Muc1, Muc16, NYESO1, VEGF-R2, PSMA and PDL1; and/or
(ii) specific to any one of ADGRE2, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CDS, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), Mucin 1 (Muc-1), Mucin 16 (Muc-16), Mesothelin (MSLN), NKCSI, NKG2D ligands, c-Met, cancer-testis antigen NYESO-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen.

13. The cell or population thereof of claim 2, wherein the cytokine signaling complex comprises:
(a) a partial or full peptide of a cell surface expressed exogenous cytokine and/or receptor thereof comprising at least one of IL2, IL4, IL6, IL7, IL9, IL10, IL11, IL12, IL15, IL18, IL21, or respective receptor(s) thereof; or
(b) at least one of:
(i) co-expression of IL15 and IL15Rα with a self-cleaving peptide in-between;
(ii) a fusion protein of IL15 and IL15Rα;
(iii) an IL15/IL15Rα fusion protein with intracellular domain of IL15Rα truncated (IL15A);
(iv) a fusion protein of IL15 and membrane bound Sushi domain of IL15Rα;
(v) a fusion protein of IL15 and IL15Rβ;
(vi) a fusion protein of TL15 and common receptor γC, wherein the common receptor γC is native or modified; and
(vii) a homodimer of IL15Rβ;
wherein any one of (b)(i)-(vii) can be co-expressed with a CAR in separate constructs or in a bi-cistronic construct; or
(c) at least one of:
(i) a fusion protein of IL7 and IL7Rα;
(ii) a fusion protein of IL7 and common receptor γC, wherein the common receptor γC is native or modified; and
(iii) a homodimer of IL7Rβ,
wherein any one of (c)(i)-(iii) is optionally co-expressed with a CAR in separate constructs or in a bi-cistronic construct;
and optionally,
(d) is transiently expressed.

14. The cell or population thereof of claim 1, wherein the derivative cell is a derivative NK or a derivative T cell, wherein the derivative NK cell is capable of recruiting and/or migrating T cells to tumor sites, and wherein the derivative NK cell or the derivative T cell is capable of reducing tumor immunosuppression in the presence of one or more checkpoint inhibitors.

15. The cell or population thereof of claim 11 or 14, wherein the one or more checkpoint inhibitors are antagonists to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, retinoic acid receptor alpha (Rara), TLR3, VISTA, NKG2A/HLA-E, or inhibitory KIR.

16. The cell or population thereof of claim 15, wherein the one or more checkpoint inhibitors comprise:
(a) one or more of atezolizumab, avelumab, durvalumab, ipilimumab, IPH4102, IPH43, IPH33, lirimumab, monalizumab, nivolumab, pembrolizumab, and their derivatives or functional equivalents; or
(b) at least one of atezolizumab, nivolumab, and pembrolizumab.

17. The cell or population thereof of claim 1, wherein the cell comprises:
(i) one or more exogenous polynucleotides integrated in one safe harbor locus or locus intended for disruption; or
(ii) more than two exogenous polynucleotides integrated in different safe harbor loci or loci intended for disruption.

18. The cell or population thereof of claim 17, wherein the safe harbor locus or loci comprises at least one of AAVS1, CCR5, ROSA26, collagen, HTRP, H11, GAPDH, TCR or RUNX1; or wherein the gene locus or loci intended for disruption comprises at least one of B2M, TAP1, TAP2, tapasin, NLRC5, CIITA, RFXANK, RFX5, RFXAP, TCRα or TCRβ constant region, NKG2A, NKG2D, CD38, CD25, CD69, CD71, CD44, CD58, CD54, CD56, CIS, CBL-B, SOCS2, PD1, CTLA4, LAG3, TIM3, or TIGIT.

19. The cell or population thereof of claim 1, wherein the cytoplasmic signaling domain of the Fas redirector comprises:
(a) a full or partial length of the intracellular domain (ICD) of MyD88 and CD40, represented by SEQ ID NO: 2; or
(b) a full or partial length of the intracellular domain (ICD) of CD27, represented by SEQ ID NO: 3; or
(c) a full or partial length of the intracellular domain (ICD) of OX40, represented by SEQ ID NO: 5.

20. The cell or population thereof of claim 19, further comprising an exogenous polynucleotide encoding a fusion protein of a partial or full peptide of IL7 and a partial or full peptide of IL7Rα.

21. A composition comprising the cell or population thereof of any one of the claims 1-20.

22. The composition of claim 21, further comprising one or more therapeutic agents.

23. The composition of claim 22, wherein the one or more therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (TMiD).

24. The composition of claim 23, wherein:
(i) the checkpoint inhibitor comprises:
(a) one or more antagonists to checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxp1, GARP, HVEM, IDO, EDO, TDO, LAIR-1, MICA/B, NR4A2, MAFB, OCT-2, retinoic acid receptor alpha (Rara), TLR3, VISTA, NKG2A/ILA-E, or inhibitory KIR;
(b) one or more of atezolizumab, avelumab, durvalumab, ipilimumab, IPH4102, IPH43, IPH33, lirimumab, monalizumab, nivolumab, pembrolizumab, and their derivatives or functional equivalents;
(c) at least one of atezolizumab, nivolumab, and pembrolizumab; or
(ii) the therapeutic agents comprise one or more of venetoclax, azacitidine, and pomalidomide.

25. The composition of claim 23, wherein the antibody comprises:
(a) an anti-CD20 antibody, an anti-HER2 antibody, an anti-CD52 antibody, an anti-EGFR antibody, an anti-CD123 antibody, an anti-GD2 antibody, or an anti-PDL1 antibody; or
(b) one or more of rituximab, veltuzumab, ofatumumab, ublituximab, ocaratuzumab, obinutuzumab, trastuzumab, pertuzumab, alemtuzumab, cetuximab, dinutuximab, avelumab, daclizumab, basiliximab, M-A251, 2A3, BC69, 24204, 22722, 24212, MAB23591, FN50, 298614, AF2359, CY1G4, DF1513, bivatuzumab, RG7356, G44-26, 7G3, CSL362, elotuzumab, and their humanized or Fc modified variants or fragments and their functional equivalents and biosimilars thereof.

26. The composition of claim 23, wherein the engager comprises:
(i) a bispecific T cell engager (BiTE);
(ii) a bispecific killer cell engager (BiKE); or
(iii) a tri-specific killer cell engager (TriKE); or
wherein the engager comprises:
(a) a first binding domain recognizing an extracellular portion of CD3, CD5, CD16, CD28, CD32, CD33, CD64, CD89, NKG2C, NKG2D, or any functional variants thereof of the cell or a by-stander immune effector cell; and
(b) a second binding domain specific to an antigen comprising any one of: B7H3, BCMA, CD10, CD19, CD20, CD22, CD24, CD30, CD33, CD34, CD38, CD44, CD52, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, Muc1, Muc16, PDL1, PSMA, PAMA, P-cadherin, ROR1, or VEGF-R2.

27. Therapeutic use of the composition of any one of the claims 21-26 by introducing the composition to a subject in need of an adoptive cell therapy, wherein the subject has an autoimmune disorder, a hematological malignancy, a solid tumor, cancer, or a virus infection.

28. A master cell bank (MCB) comprising the clonal iPSC of any one of the claims 1-20.

29. A method of manufacturing the derivative cell of any one of the claims 1-20, wherein the derivative cell is an effector cell, and the method comprises:
differentiating a genetically engineered iPSC, wherein the genetically engineered iPSC comprises the exogenous polynucleotide encoding a Fas redirector that redirects Fas signaling upon binding to a FAS agonist,
thereby providing the effector cell with improved apoptosis resistance and/or exhaustion resistance.

30. The method of claim 29, wherein the Fas redirector comprises:
(a) an extracellular Fas binding domain comprising a partial or full peptide of an extracellular domain (ECD) of a Fas receptor (FAS), or a variant or an allele thereof, and
(b) a cytoplasmic signaling domain comprising a partial or full peptide of an intracellular domain (ICD) of one or more co-stimulation molecules; and
wherein the genetically engineered iPSC is a single cell, a clonal cell, or a cell line cell.

31. The method of claim 30, wherein:
(i) the Fas redirector further comprises a transmembrane region comprising a transmembrane domain, or a portion thereof, of a transmembrane protein;
(ii) the one or more co-stimulation molecules comprise CD27, CD28, CD40, MyD88, OX40, IL12Rβ2, IL18R1, IL21R, or a combination thereof,
(iii) the one or more co-stimulation molecules do not comprise 41BB;
(iv) the FAS agonist comprises a Fas ligand (FasL); or
(v) the genetically engineered iPSC comprising the Fas redirector further comprises one or more of:
(a) an exogenous polynucleotide encoding a chimeric antigen receptor (CAR);
(b) an exogenous polynucleotide encoding a CD16 or a variant thereof,
(c) CD38 knockout; and
(d) an exogenous polynucleotide encoding a cytokine signaling complex comprising a partial or full peptide of a cell surface expressed exogenous cytokine and/or a receptor thereof.

32. The method of claim 31, wherein the transmembrane region of the Fas redirector comprises:
(i) a full length or at least a portion of the native or modified transmembrane region of FAS, CD2, CD3δ, CD3ε, CD3γ, CD3ζ, CD4, CD8, CD8a, CD8b, CD16, CD27, CD28, CD28H, CD40, CD84, CD166, 4-1BB, OX40, ICOS, ICAM-1, CTLA4, PD1, LAG3, 2B4, BTLA, DNAM1, DAP10, DAP12, FcERIγ, IL7, IL12, IL15, KIR2DL4, KIR2DS1, KIR2DS2, NKp30, NKp44, NKp46, NKG2C, NKG2D, CS1, or a T cell receptor polypeptide; or
(ii) a full or partial length of the transmembrane domain of FAS.

33. The method of claim 31, wherein:
(i) the extracellular binding domain of the Fas redirector comprises a sequence having at least about 85%, about 90%, about 95%, about 96%, about 97%, about 98%, or about 99% identity to SEQ ID NO: 1;
(ii) the cytoplasmic signaling domain of the Fas redirector comprises:
(a) a full or partial length of the intracellular domain (ICD) of MyD88 and CD40, represented by SEQ ID NO: 2; or
(b) a full or partial length of the intracellular domain (ICD) of CD27, represented by SEQ ID NO: 3; or
(c) a full or partial length of the intracellular domain (ICD) of CD28, represented by SEQ ID NO: 4; or
(d) a full or partial length of the intracellular domain (ICD) of OX40, represented by SEQ ID NO: 5; or
(e) a full or partial length of the intracellular domain (ICD) of IL12Rβ2, represented by SEQ ID NO: 6; or
(f) a full or partial length of the intracellular domain (ICD) of IL18R1, represented by SEQ ID NO: 7; or
(g) a full or partial length of the intracellular domain (ICD) of IL21R, represented by SEQ ID NO: 8; or
(iii) the Fas redirector comprises an amino acid sequence that has at least about 85%, about 90%, about 95%, about 96%, about 97%, about 98%, or about 99% identity to any one of SEQ ID Nos: 9-15.

34. The method of any one of claims 29-33, wherein the genetically engineered iPSC further comprises one or more edits resulting in:
(i) at least one of the genotypes listed in Table 1;
(ii) HLA-I deficiency and/or HLA-II deficiency;
(iii) introduction of HLA-G or non-cleavable HLA-G, or knockout of one or both of CD58 and CD54;
(iv) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; and/or
(v) introduction of at least one of HLA-E, 4-1BBL, CD3, CD4, CD8, CD47, CD113, CD131, CD137, CD80, PDL1, A_{2A}R, antigen-specific TCR, chimeric fusion receptor (CFR), Fc receptor, an antibody or functional variant or fragment thereof, a checkpoint inhibitor, an engager, and surface triggering receptor for coupling with bi- or multi-specific or universal engagers.

35. The method of any one of claims 29-34, wherein the cell comprises HLA-I deficiency, and/or HLA-II deficiency; and optionally,
wherein the cell comprises an exogenous polynucleotide encoding HLA-G, HLA-E, or a variant thereof, or comprises deletion or disruption of one or both of CD54 and CD58.

36. The method of claim 31, further comprising:
genomically engineering an iPSC to knock in: (a) the polynucleotide encoding the signaling redirector receptor; and optionally, (b) the exogenous polynucleotide encoding the chimeric antigen receptor (CAR); and optionally (c) the exogenous polynucleotide encoding the CD16 or a variant thereof, and optionally further comprising genomically engineering the iPSC:
(i) to knock out CD38,
(ii) to knock out one or both of B2M and CIITA,
(iii) to knock out one or both of CD58 and CD54, and/or
(iv) to introduce HLA-G or non-cleavable HLA-G and/or the signaling complex comprising the partial or full peptide of the cell surface expressed exogenous cytokine and/or receptor thereof.

37. The method of claim 36, wherein the genomic engineering comprises targeted editing.

38. The method of claim 37, wherein the targeted editing comprises deletion, insertion, or in/del, and wherein the targeted editing is carried out by CRISPR, ZFN, TALEN, homing nuclease, homology recombination, or any other functional variation of these methods.

39. A method of improving effector cell durability or preventing cell death of effector cells in an adoptive cell therapy provided to a subject in need thereof by obtaining the effector cells according to any one of claims 29-38.

40. A method of improving efficacy of an adoptive cell therapy provided to a subject in need thereof, the method comprising administering the effector cells to the subject, wherein the effector cells comprise the derivative cell or population thereof of any one the claims 1-20.

41. The method of claim 40, further comprising administering one or more therapeutic agents to the subject.

42. The method of claim 41, wherein the one or more therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD).

43. The method of claim 42, wherein:
(i) the checkpoint inhibitor comprises:
(a) one or more antagonists to checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxp1, GARP, HVEM, IDO, EDO, TDO, LAIR-1, MICA/B, NR4A2, MAFB, OCT-2, retinoic acid receptor alpha (Rara), TLR3, VISTA, NKG2A/HLA-E, or inhibitory KIR;
(b) one or more of atezolizumab, avelumab, durvalumab, ipilimumab, IPH4102, IPH43, IPH33, lirimumab, monalizumab, nivolumab, pembrolizumab, and their derivatives or functional equivalents; or
(c) at least one of atezolizumab, nivolumab, and pembrolizumab; or
(ii) the one or more therapeutic agents comprise one or more of venetoclax, azacitidine, and pomalidomide.

44. A method of treating a subject in need of an adoptive cell therapy, wherein the method comprises infusing the subject with effector cells, wherein the effector cells comprise the derivative cell or population thereof according to any one of claims 1-20 in combination with an exogenous cytokine.


SEQUENCE LISTING (26 sequences)

SEQ ID NO: 1 - protein, 148 aa, synthetic construct
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 2 - protein, 220 aa, synthetic construct
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPKKVAKKPTNKAPHPKQEPQEINFPDDLPGSNTAAPVQETLHGCQPVTQEDGKESRISVQERQ

SEQ ID NO: 3 - protein, 48 aa, synthetic construct
QRRKYRSNKGESPVEPAEPCHYSCPREEEGSTIPIQEDYRKPEPACSP

SEQ ID NO: 4 - protein, 41 aa, synthetic construct
RSKRSRLLHSDYMNMTPRRPGPTRKHYQPYAPPRDFAAYRS

SEQ ID NO: 5 - protein, 42 aa, synthetic construct
ALYLLRRDQRLPPDAHKPPGGGSFRTPIQEEQADAHSTLAKI

SEQ ID NO: 6 - protein, 51 aa, synthetic construct
SDPKPENPACPWTVLPAGDLPTHDGYLPSNIDDLPSHEAPLADSLEELEPQ

SEQ ID NO: 7 - protein, 191 aa, synthetic construct
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 8 - protein, 285 aa, synthetic construct
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 9 - protein, 410 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPKKVAKKPTNKAPHPKQEPQEINFPDDLPGSNTAAPVQETLHGCQPVTQEDGKESRISVQERQ

SEQ ID NO: 10 - protein, 238 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVQRRKYRSNKGESPVEPAEPCHYSCPREEEGSTIPIQEDYRKPEPACSP

SEQ ID NO: 11 - protein, 231 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVRSKRSRLLHSDYMNMTPRRPGPTRKHYQPYAPPRDFAAYRS

SEQ ID NO: 12 - protein, 232 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVALYLLRRDQRLPPDAHKPPGGGSFRTPIQEEQADAHSTLAKI

SEQ ID NO: 13 - protein, 241 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVSDPKPENPACPWTVLPAGDLPTHDGYLPSNIDDLPSHEAPLADSLEELEPQ

SEQ ID NO: 14 - protein, 381 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 15 - protein, 475 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 16 - protein, 153 aa, synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 17 - protein, 219 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 18 - protein, 263 aa, synthetic construct
SNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 308 aa, synthetic construct
TTTPAPRPPTPAPTIASQPLSLRPEACRPAAGGAVHTRGLDFACDSNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 340 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHYQVSFCLVMVLLFAVDTGLYFSVKTNIRSSTRDWKDHKFKWRKDPQDK

SEQ ID NO: 21 - protein, 336 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLFFPPGYQVSFCLVMVLLFAVDTGLYFSVKTNIRSSTRDWKDHKFKWRKDPQDK

SEQ ID NO: 22 - protein, 335 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDK

SEQ ID NO: 23 - protein, 379 aa, synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQ

SEQ ID NO: 24 - protein, 375 aa, synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYL

SEQ ID NO: 25 - protein, 242 aa, synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIR

SEQ ID NO: 26 - protein, 635 aa, synthetic construct
MDWTWILFLVAAATRVHSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGSGGGGSGGGGSGGGGSGGGSLQESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ